(19) United States
(12) Patent Application Publication
(10) Pub. No.: US 2024/0374712 A1
(43) Pub. Date: Nov. 14, 2024

(54) SARS-COV-2 VACCINE COMPOSITIONS

(71) Applicant: Novavax, Inc., Gaithersburg, MD (US)

(72) Inventors: Gale SMITH, Germantown, MD (US); Nita PATEL, Gaithersburg, MD (US)

(21) Appl. No.: 18/645,259

(22) Filed: Apr. 24, 2024

Related U.S. Application Data
(60) Provisional application No. 63/497,986, filed on Apr. 24, 2023.
(60) Provisional application No. 63/507,079, filed on Jun. 8, 2023.
(60) Provisional application No. 63/507,412, filed on Jun. 9, 2023.
(60) Provisional application No. 63/508,088, filed on Jun. 14, 2023.
(60) Provisional application No. 63/579,438, filed on Aug. 29, 2023.
(60) Provisional application No. 63/580,596, filed on Sep. 5, 2023.

(51) Int. Cl. A61K 39/215; A61K 39/00; A61P 31/14; C12N 7/00

(52) U.S. Cl. CPC A61K 39/215; A61P 31/14; C12N 7/00; A61K 2039/53; A61K 2039/545; A61K 2039/55555; A61K 2039/55577; A61K 2039/575; C12N 2770/20022; C12N 2770/20034

(57) ABSTRACT

Disclosed herein are coronavirus (CoV) Spike(S) polypeptides, including naturally and non-naturally occurring polypeptides, and nanoparticles and immunogenic compositions comprising the same, which are useful for stimulating immune responses against various SARS-COV-2 strains. The nanoparticles present antigens from pathogens surrounded to and associated with a detergent core resulting in enhanced stability and good immunogenicity. Dosages, formulations, and methods for preparing the vaccines and nanoparticles are also disclosed.

### CROSS REFERENCE TO RELATED APPLICATIONS

This application claims priority from U.S. Provisional Application Nos. 63/497,986, filed Apr. 24, 2023, 63/507,079 filed Jun. 8, 2023, 63/507,412 filed Jun. 9, 2023, 63/508,088 filed Jun. 14, 2023, 63/579,438 filed Aug. 29, 2023, and 63/580,596 filed on Sep. 5, 2023, the entire contents of which are hereby incorporated by reference.

### DESCRIPTION OF THE TEXT FILE SUBMITTED ELECTRONICALLY

The contents of the electronic sequence listing (1450_103US1_Sequence_Listing_04_24_2024.xml; Size: 817,457 bytes; and Date of Creation: Apr. 24, 2024) are herein incorporated by reference in its entirety.

### FIELD

The present disclosure is generally related to coronavirus (CoV) Spike(S) polypeptides, including naturally and non-naturally occurring polypeptides, and nanoparticles and immunogenic compositions comprising the same, which are useful for stimulating immune responses. The nanoparticles provide antigens, for example, glycoprotein antigens, optionally associated with a detergent core and are typically produced using recombinant approaches. The nanoparticles have improved stability and enhanced epitope presentation. The disclosure also provides compositions containing the nanoparticles, methods for producing them, and methods of stimulating immune responses.

### BACKGROUND OF THE INVENTION

Infectious diseases remain a problem throughout the world. While progress has been made on developing vaccines against some pathogens, many remain a threat to human health. The outbreak of sudden acute respiratory syndrome coronavirus 2 (SARS-COV-2) has caused 6.5 million deaths worldwide. The SARS-COV-2 coronavirus belongs to the same family of viruses as severe acute respiratory syndrome coronavirus (SARS-COV) and Middle East respiratory syndrome coronavirus (MERS-COV), which have killed hundreds of people in the past 17 years. SARS-COV-2 causes the disease COVID-19.

The development of vaccines that prevent or reduce the severity of life-threatening infectious diseases like the SARS-COV-2 coronavirus is desirable. However, human vaccine development remains challenging because of the highly sophisticated evasion mechanisms of pathogens and difficulties stabilizing vaccines. The development of a vaccine that induces protection against current SARS-COV-2 strains and SARS-COV-2 strains that will emerge in the future is desirable.

### SUMMARY OF THE INVENTION

The present disclosure provides CoV S polypeptides, including naturally and non-naturally occurring polypeptides, and compositions containing the same suitable for inducing immune responses against SARS-COV-2. The disclosure also provides nanoparticles and compositions containing the glycoproteins as well as methods of stimulating immune responses. The nanoparticles and compositions described herein also are suitable for inducing cross-neutralizing immune responses against Middle East Respiratory Syndrome (MERS) and Severe Acute Respiratory Syndrome (SARS).

### BRIEF DESCRIPTION OF THE DRAWINGS

FIG. 1A shows the primary structure of a wild-type SARS-COV-2 S polypeptide, containing a signal peptide, numbered with respect to SEQ ID NO: 1. FIG. 1B shows the primary structure of a wild-type SARS-COV-2 S polypeptide, without a signal peptide, numbered with respect to SEQ ID NO: 2.

FIG. 2 shows the dosage regimen of Example 7.

FIGS. 3A-3F show that Composition 3 (FIG. 3A, FIG. 3D), Composition 2 (FIG. 3B, FIG. 3E), and Composition 1 (FIG. 3C, FIG. 3F) of Example 3 each neutralized pseudoviruses expressing S proteins from various SARS-COV-2 heterogeneous strains. Open circles of FIGS. 3D-3F represent individual data points, solid bars represent group geometric mean titers, error bars represent 95% confidence intervals, and the horizontal dashed line represents the assay limit of detection (LOD).

FIGS. 4A-4D show that administration of two doses of Composition 2 (FIG. 4A, FIG. 4C), a monovalent composition comprising a CoV S glycoprotein of SEQ ID NO: 274, results in a superior immune response in mice to administration of two doses of Composition 4 (FIG. 4B, FIG. 4D), which is a bivalent composition containing a CoV S glycoprotein of SEQ ID NO: 274 and a CoV S glycoprotein of SEQ ID NO: 87. Open circles of FIGS. 4C-4D represent individual data points, solid bars represent group geometric mean titers, error bars represent 95% confidence intervals, and the horizontal dashed line represents the assay limit of detection (LOD).

FIGS. 5A-5F shows the immune response induced by a boost dose containing Composition 2 (FIG. 5B, FIG. 5E) or Composition 3 (FIG. 5C, FIG. 5F) of Example 3. Boosting with either Composition 2 (FIG. 5B, FIG. 5E) or Composition 1 (FIG. 5C, FIG. 5F) resulted in an improved immune response against pseudoviruses expressing S proteins from various SARS-COV-2 heterogeneous strains, as compared to mice that were not boosted (FIG. 5A, FIG. 5D).

FIGS. 6A-6C shows the immune response induced by a boost dose containing Composition 2 (FIG. 6B) or Composition 3 (FIG. 6C) of Example 3. Boosting with either Composition 2 (FIG. 6B) or Composition 1 (FIG. 6C) resulted in an improved immune response against pseudoviruses expressing S proteins from various SARS-COV-2 heterogeneous strains, as compared to mice that were not boosted (FIG. 6A).

FIGS. 7A-7E show the ability of serum from Rhesus macaques that are administered various immunogenic compositions to neutralize pseudovirus. FIG. 7A shows the ability of serum from Rhesus macaques administered two doses of Composition 3 and a booster dose of Composition 2 to neutralize pseudovirus. FIG. 7B shows the ability of serum from Rhesus macaques administered two doses of Composition 6 and a booster dose of Composition 2 to neutralize pseudovirus. FIG. 7C shows the ability of serum from Rhesus macaques administered two doses of Composition 5 and a booster dose of Composition 2 to neutralize pseudovirus. FIG. 7D shows the ability of serum from Rhesus macaques administered primary dose of Composition 3 followed by a booster dose of Composition 2 to neutralize pseudovirus. FIG. 7E shows the ability of serum from Rhesus macaques administered primary dose of Composition 5 followed by a booster dose of Composition 2 to neutralize pseudovirus.

FIG. 8 shows the ability of a dosage regimen comprising administering two doses of Composition 5 followed by boosting with Composition 2 to induce an immune response that blocks binding of hACE 2 to a SARS-COV-2 S glycoprotein. Compositions 2 and 5 are described in Example 3.

FIGS. 9A-9C show the ability of various dosage regimens to induce an immune response in Rhesus macaques that blocks binding of hACE 2 to a SARS-COV-2 S glycoprotein. FIG. 9A shows the immune response induced when two doses of Composition 3 are administered, followed by a boost of Composition 2. FIG. 9B shows the immune response induced when two doses of Composition 6 are administered, followed by a boost of Composition 2. FIG. 9C shows the immune response induced when two doses of Composition 5 are administered, followed by a boost of Composition 2. Compositions 2, 3, 5, and 6 are described in Example 3.

FIGS. 10A-10B shows the ratio of Th1/Th2 cytokines produced by CD4+ T cells in mice administered two doses of Composition 3 followed by a boost dose with Composition 2 (FIG. 10A), or two doses of Composition 5, followed by a boost dose of Composition 2 (FIG. 10B). Compositions 2, 3, and 5 are described in Example 3.

FIG. 11 shows the CD4+ T cell response in Rhesus macaques administered two doses of Composition 5, followed by a boost dose of Composition 2. Compositions 5 and 2 are described in Example 3.

FIG. 12 shows the dosage regimen of Example 8.

FIG. 13A shows the anti-S IgG antibodies in mice after administration of two doses of an immunogenic composition comprising the SARS-COV 2 S glycoprotein of SEQ ID NO: 274. FIG. 13B shows the anti-S IgG antibodies in mice after administration of two doses of an immunogenic composition comprising the SARS-COV 2 S glycoprotein of SEQ ID NO: 260. Additional experimental details are found in Example 8.

FIG. 14A shows that administration of two doses of an immunogenic composition comprising the SARS-COV 2 S glycoprotein of SEQ ID NO: 274 results in antibodies that block binding of SARS-COV-2 to hACE2. FIG. 14B shows that administration of two doses of an immunogenic composition comprising the SARS-COV 2 S glycoprotein of SEQ ID NO: 260 results in antibodies that block binding of SARS-COV-2 to hACE2. Additional experimental details are found in Example 8.

FIG. 15A and FIG. 16B show that administration of two doses of an immunogenic composition comprising the SARS-COV 2 S glycoprotein of SEQ ID NO: 274 results in neutralization of pseudoviruses expressing SARS-COV-2 S glycoproteins. FIG. 15B and FIG. 16C show that administration of two doses of an immunogenic composition comprising the SARS-COV 2 S glycoprotein of SEQ ID NO: 260 results in neutralization of pseudoviruses expressing SARS-CoV-2 S glycoproteins. Additional experimental details are found in Example 8.

FIG. 16A shows the neutralizing antibody titer resulting from administering two doses of an immunogenic composition comprising the SARS-COV 2 S glycoprotein of SEQ ID NO: 87. FIG. 16B shows that administration of two doses of an immunogenic composition comprising the SARS-COV 2 S glycoprotein of SEQ ID NO: 274 results in neutralization of pseudoviruses expressing SARS-COV-2 S glycoproteins. FIG. 16C shows that administration of two doses of an immunogenic composition comprising the SARS-COV 2 S glycoprotein of SEQ ID NO: 260 results in neutralization of pseudoviruses expressing SARS-COV-2 S glycoproteins. Additional experimental details are found in Example 8.

FIG. 17 shows the dosage regimen of Example 9.

FIG. 18A shows the anti-S IgG antibodies in serum of mice after administration of two doses of an immunogenic composition comprising the SARS-COV 2 S glycoprotein of SEQ ID NO: 274. FIG. 18B shows the neutralizing antibodies present in the serum of mice after administration of two doses of an immunogenic composition comprising the SARS-COV 2 S glycoprotein of SEQ ID NO: 274. Additional experimental details are found in Example 9.

FIG. 19A shows the antigenic cartography of neutralizing responses induced by immunizing with two doses of an immunogenic composition comprising the SARS-COV 2 S glycoprotein of SEQ ID NO: 274. FIG. 19B shows the antigenic cartography of neutralizing responses induced by boosting with an immunogenic composition comprising the SARS-COV 2 S glycoprotein of SEQ ID NO: 274. FIG. 19C shows the antigenic cartography of neutralizing responses induced by boosting with an immunogenic composition comprising the SARS-COV 2 S glycoprotein of SEQ ID NO: 260. Additional experimental details are found in Example 9.

FIG. 20A shows pseudovirus neutralizing titers induced by administering a SARS-COV-2 S glycoprotein of SEQ ID NO: 274 in the primary series. FIG. 20B shows pseudovirus neutralizing titers induced by administering a SARS-COV-2 S glycoprotein of SEQ ID NO: 274 in the one month boost. FIG. 20C shows pseudovirus neutralizing titers induced by administering a SARS-CoV-2 S glycoprotein of SEQ ID NO: 260 in the one month boost. Additional experimental details are found in Example 9.

FIG. 21 shows the dosage regimen of Example 10.

FIG. 22A shows the anti-S IgG antibodies in serum of macaques after administration of a first dose and a second dose containing the SARS-COV 2 S glycoprotein of SEQ ID NO: 87 and administration of a third dose containing a SARS-COV 2 S glycoprotein of SEQ ID NO: 274. FIG. 22B shows the anti-S IgG antibodies in serum of macaques after administration of a first dose and a second dose containing the SARS-COV 2 S glycoprotein of SEQ ID NO: 222 and administration of a third dose containing a SARS-COV 2 S glycoprotein of SEQ ID NO: 274. FIG. 22C shows the anti-S IgG antibodies in serum of macaques after administration of a first dose and a second dose containing the SARS-COV 2 S glycoprotein of SEQ ID NO: 87 and the SARS-COV-2 S glycoprotein of SEQ ID NO: 222 and administration of a third dose containing a SARS-COV 2 S glycoprotein of SEQ ID NO: 274. Additional experimental details are found in Example 10.

FIG. 23 shows the anti-S IgG antibodies in serum of macaques after administration of a first dose and a second dose containing the SARS-COV 2 S glycoprotein of SEQ ID NO: 274. FIG. 24 shows pseudovirus neutralizing titers induced by administering a SARS-COV-2 S glycoprotein of SEQ ID NO: 274 in the primary series. Additional experimental details are found in Example 10.

FIG. 25 shows the CD4+ T cell response of macaques administered a first dose and a second dose containing the SARS-COV 2 S glycoprotein of SEQ ID NO: 274. Additional experimental details are found in Example 10.

FIG. 26 shows the multifunctional antigen specific CD4+ T cell response of macaques administered a first dose and a second dose containing the SARS-COV 2 S glycoprotein of SEQ ID NO: 274. Additional experimental details are found in Example 10.

FIG. 27 shows the dosage regimen of Example 11.

FIG. 28A shows the anti-rS IgG antibodies in serum of mice after administration of two doses of an immunogenic composition comprising the SARS-COV 2 S glycoprotein of SEQ ID NO: 274. FIG. 28B shows the neutralizing antibodies present in the serum of mice after administration of two doses of an immunogenic composition comprising the SARS-COV 2 S glycoprotein of SEQ ID NO: 329. Additional experimental details are found in Example 11.

FIG. 29A shows pseudovirus neutralizing titers induced by administering a SARS-COV-2 S glycoprotein of SEQ ID NO: 274 in the primary series. FIG. 29B shows pseudovirus neutralizing titers induced by administering a SARS-COV-2 S glycoprotein of SEQ ID NO: 329 in the primary series. Additional experimental details are found in Example 11.

FIG. 30 shows pseudovirus neutralizing titers and antigenic distances induced by administering a SARS-COV-2 S glycoprotein of SEQ ID NO: 274 in the primary series against different SARS-COV-2 variant strains, with low titers for JN.1 and JN. 1 subvariants. Additional experimental details are found in Example 11.

FIG. 31 shows pseudovirus neutralizing titers and antigenic distances induced by administering a SARS-COV-2 S glycoprotein of SEQ ID NO: 329 in the primary series against different SARS-COV-2 variant strains, with high titers for JN.1 and JN.1 subvariants. Additional experimental details are found in Example 11.

FIGS. 32A-32D show the anti-rS IgG antibodies in serum of mice after administration of Composition 2 in the primary series (FIG. 32A), compared to the anti-rS IgG antibodies in scrum of mice prior to administration of a boost dose (FIG. 32B), against the anti-rS IgG antibodies in serum of mice induced after administration of a boost dose containing Composition 2 (FIG. 32C) or Composition 8 (FIG. 32D). Additional experimental details are found in Example 11.

FIGS. 33A-33C show pseudovirus neutralizing titers and antigenic distances induced by administering a SARS-COV-2 S glycoprotein of SEQ ID NO: 274 in the primary series followed by a boost dose containing Composition 8 (FIG. 33C) of Example 11. Boosting with Composition 8 (FIG. 33C) resulted in an improved immune response against pseudoviruses expressing S proteins from various SARS-COV-2 heterogeneous strains, as compared to pre-boosted mice (FIG. 33B) who received a primary dose of Composition 2 (FIG. 33A). Additional experimental details arc found in Example 11.

FIGS. 34A-34B show pseudovirus neutralizing titers and antigenic distances induced by administering Composition 2 (FIG. 34A) in the primary series followed by a boost dose after 2 months containing Composition 8 (FIG. 34B) of Example 11. Boosting with Composition 8 (FIG. 34B) elicited an improved immune response against pseudoviruses expressing S proteins from various SARS-COV-2 heterogeneous strains (FIG. 34A). Additional experimental details are found in Example 11.

FIGS. 35A-35B show pseudovirus neutralizing titers and antigenic distances induced by administering Composition 7 (FIG. 35A) in the primary series followed by a boost dose after 2 months containing Composition 8 (FIG. 35B) of Example 11. Boosting with Composition 8 (FIG. 35B) elicited an improved immune response against pseudoviruses expressing S proteins from various SARS-COV-2 heterogeneous strains (FIG. 35A). Additional experimental details are found in Example 11.

FIG. 36 shows the ratio of Th1/Th2 cytokines produced by CD4+ T cells in mice administered with doses of Composition 2 followed by a boost dose at 2 months with Composition 2. Additional experimental details are found in Example 11.

FIG. 37 shows the ratio of Th1/Th2 cytokines produced by CD4+ T cells in mice administered with doses of Composition 2 followed by a boost dose at 2 months with Composition 8. Additional experimental details are found in Example 11.

FIGS. 38A-38B show the Triple_Th 1 CD4+ T cell response in mice administered with doses of Composition 2 followed by a boost dose at 2 months with Composition 2 (FIG. 38A) and in mice administered with doses of Composition 2 followed by a boost dose at 2 months with Composition 8 (FIG. 38B). Additional experimental details are found in Example 11.

FIGS. 39A-39B show the CD4+ T cell IFN-γ response in mice administered with doses of Composition 2 followed by a boost dose at 2 months with Composition 2 (FIG. 39A) and in mice administered with doses of Composition 2 followed by a boost dose at 2 months with Composition 8 (FIG. 39B). Additional experimental details are found in Example 11.

FIGS. 40A-40B show the CD8+ T cell Th1 responses in mice administered with doses of Composition 2 followed by a boost dose at 2 months with Composition 2 (FIG. 40A) and in mice administered with doses of Composition 2 followed by a boost dose at 2 months with Composition 8 (FIG. 40B). Additional experimental details are found in Example 11.

FIG. 41A shows T follicular helper cell levels (Tfh) in mice administered with doses of Composition 2 followed by a boost dose at 2 months with Composition compared to in mice administered with doses of Composition 2 followed by a boost dose at 2 months with Composition 8. FIG. 41B shows Germinal Center (GC) B cell levels in mice administered with doses of Composition 2 followed by a boost dose at 2 months with Composition compared to in mice administered with doses of Composition 2 followed by a boost dose at 2 months with Composition 8.

FIG. 42 shows the dosage regimen of Example 12.

FIG. 43A shows the anti-rS responses in primed Rhesus macaques prior to administration of a second booster dose of an immunogenic composition comprising the SARS-COV 2 S glycoprotein of SEQ ID NO: 329. FIG. 43B shows the anti-rS responses in primed Rhesus macaques after administration of a second booster dose of an immunogenic composition comprising the SARS-COV 2 S glycoprotein of SEQ ID NO: 329. Additional experimental details are found in Example 12.

FIG. 44A shows pseudovirus neutralizing titers and antigenic distances in primed Rhesus macaques prior to administration of a second booster dose of an immunogenic composition comprising the SARS-COV 2 S glycoprotein of SEQ ID NO: 329. FIG. 44B shows pseudovirus neutralizing titers and antigenic distances in primed Rhesus macaques after administration of a second booster dose of an immunogenic composition comprising the SARS-COV 2 S glycoprotein of SEQ ID NO: 329. A second booster dose of an immunogenic composition comprising the SARS-COV 2 S glycoprotein of SEQ ID NO: 329 elicited pseudovirus neutralizing antibodies against different SARS-COV-2 variant strains. Additional experimental details are found in Example 12.

FIG. 45 shows the CD4+ T cell IFN-γ response in primed Rhesus macaques after administration of a second booster dose of an immunogenic composition comprising the SARS-CoV 2 S glycoprotein of SEQ ID NO: 329. A second booster dose of an immunogenic composition comprising the SARS-COV 2 S glycoprotein of SEQ ID NO: 329 elicited a Th1-biased cellular response and conserved T-cell epitopes recognized for all variants tested. Additional experimental details are found in Example 12.

FIG. 46 shows the multifunctional antigen specific CD4+ T cell response of primed Rhesus macaques after administration of a second booster dose of an immunogenic composition comprising the SARS-COV 2 S glycoprotein of SEQ ID NO: 329. Additional experimental details are found in Example 12.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein, the term “about” or “approximately” when preceding a numerical value indicates the value plus or minus a range of 10%. For example, “about 100” encompasses 90 and 110.

As used herein, the terms “immunogen,” “antigen,” and “epitope” refer to substances such as proteins, including glycoproteins, and peptides that are capable of eliciting an immune response.

As used herein, an “immunogenic composition” is a composition that comprises an antigen where administration of the composition to a subject results in the development in the subject of a humoral and/or a cellular immune response to the antigen.

As used herein, a “subunit” composition, for example a vaccine, that includes one or more selected antigens but not all antigens from a pathogen. Such a composition is substantially free of intact virus or the lysate of such cells or particles and is typically prepared from at least partially purified, often substantially purified immunogenic polypeptides from the pathogen. The antigens in the subunit composition disclosed herein are typically prepared recombinantly, often using a baculovirus system.

As used herein, “substantially” refers to isolation of a substance (e.g. a compound, polynucleotide, or polypeptide) such that the substance forms the majority percent of the sample in which it is contained. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an immunogen derived from a pathogen, which is used to induce an immune response against the pathogen that provides protective immunity (e.g., immunity that protects a subject against infection with the pathogen and/or reduces the severity of the disease or condition caused by infection with the pathogen). The protective immune response may include formation of antibodies and/or a cell-mediated response. Depending on context, the term “vaccine” may also refer to a suspension or solution of an immunogen that is administered to a subject to produce protective immunity.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In aspects, the adults are seniors about 65 years or older, or about 60 years or older. In aspects, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

In aspects, the subject is immunocompromised. In embodiments, the immunocompromised subject is administered a medication that causes immunosuppression. Non-limiting examples of medications that cause immunosuppression include corticosteroids (e.g., prednisone), alkylating agents (e.g., cyclophosphamide), antimetabolites (e.g., azathioprine or 6-mercaptopurine), transplant-related immunosuppressive drugs (e.g., cyclosporine, tacrolimus, sirolimus, or mycophenolate mofetil), mitoxantrone, chemotherapeutic agents, methotrexate, tumor necrosis factor (TNF)-blocking agents (e.g., etanercept, adalimumab, infliximab). In embodiments, the immunocompromised subject is infected with a virus (e.g., human immunodeficiency virus or Epstein-Barr virus). In embodiments, the virus is a respiratory virus, such as respiratory syncytial virus, influenza, parainfluenza, adenovirus, or a picornavirus. In embodiments, the immunocompromised subject has acquired immunodeficiency syndrome (AIDS). In embodiments, the immunocompromised subject is a person living with human immunodeficiency virus (HIV). In embodiments, the immunocompromised subject is immunocompromised due to a treatment regiment designed to prevent inflammation or prevent rejection of a transplant. In embodiments, the immunocompromised subject is a subject who has received a transplant. In embodiments, the immunocompromised subject has undergone radiation therapy or a splenectomy. In embodiments, the immunocompromised subject has been diagnosed with cancer, an autoimmune disease, tuberculosis, a substance use disorder (e.g., an alcohol, opioid, or cocaine use disorder), stroke or cerebrovascular disease, a solid organ or blood stem cell transplant, sickle cell disease, thalassemia, autoimmune lymphoproliferative syndrome (ALPS), autoimmune polyglandular syndrome type 1 (APS-1), B-cell expansion with NF-κB and T-cell anergy (BENTA) disease, capsase eight deficiency state (CEDS), chronic granulomatous disease (CGD), common variable immunodeficiency (CVID), congenital neutropenia syndromes, a deficiency in the cytotoxic T-lymphocyte-associated antigen 4 (CTLA-4), a DOCK8 deficiency, a GATA2 deficiency, a glycosylation disorder with immunodeficiency, a hyper-immunoglobulin E syndrome (HIES), hyper-immunoglobulin M syndrome, diabetes, type 1 diabetes, type 2 diabetes, interferon gamma deficiency, interleukin 12 deficiency, interleukin 23 deficiency, leukocyte adhesion deficiency, lipopolysaccharide-responsive beige-like anchor (LRBA) deficiency, PI3 kinase disease, PLCG2-associated antibody deficiency and immune dysregulation (PLAID), severe combined immunodeficiency (SCID), STAT3 dominant-negative disease, STAT3 gain-of-function disease, warts, hypogammaglobulinemia, infections, and myelokathexis (WHIM) syndrome, Wisckott-Aldrich syndrome (WAS), X-linked agammaglobulinemia (XLA), X-linked lymphoproliferative disease (XLP), uremia, malnutrition, or XMEN disease. In embodiments, the immunocompromised subject is a current or former cigarette smoker. In embodiments, the immunocompromised subject has a B-cell defect, T-cell defect, macrophage defect, cytokine defect, phagocyte deficiency, phagocyte dysfunction, complement deficiency or a combination thereof.

In embodiments, the subject is overweight or obese. In embodiments, an overweight subject has a body mass index (BMI)≥25 kg/m2 and <30 kg/m2. In embodiments, an obese subject has a BMI that is >30 kg/m2. In embodiments, the subject has a mental health condition. In embodiments, the mental health condition is depression, schizophrenia, or anxiety.

As used herein, the term “pharmaceutically acceptable” means being approved by a regulatory agency of a U.S. Federal or a state government or listed in the U.S. Pharmacopeia, European Pharmacopeia or other generally recognized pharmacopcia for use in mammals, and more particularly in humans. These compositions can be useful as a vaccine and/or antigenic compositions for inducing a protective immune response in a vertebrate.

As used herein, the term “NVX-CoV2373” refers to a vaccine composition comprising the BV2373 Spike glycoprotein (SEQ ID NO: 87) and Fraction A and Fraction C iscom matrix (e.g., MATRIX-M™).

As used herein, the term “modification” as it refers to a CoV S polypeptide refers to mutation, deletion, or addition of one amino acid of the CoV S polypeptide. The location of a modification within a CoV S polypeptide can be determined based on aligning the sequence of the polypeptide to SEQ ID NO: 1 (CoV S polypeptide containing signal peptide) or SEQ ID NO: 2 (mature CoV S polypeptide lacking a signal peptide).

The term SARS-COV-2 “variant”, used interchangeably herein with a “heterogeneous SARS-COV-2 strain,” refers to a SARS-COV-2 virus comprising a CoV S polypeptide having one or more modifications as compared to a SARS-COV S polypeptide having the amino acid sequence of SEQ ID NO: 1 or SEQ ID NO: 2. For example, a SARS-COV-2 variant may have at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, or at least about 35 modifications, as compared to a CoV S polypeptide having the amino acid sequence of SEQ ID NO: 1 or SEQ ID NO: 2. For example, a SARS-COV-2 variant may have at least one and up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, up to 30, up to 31, up to 32, up to 33, up to 34, up to 35 modifications, up to 40 modifications, up to 45 modifications, up to 50 modifications, up to 55 modifications, up to 60 modifications, up to 65 modifications, up to 70 modifications, up to 75 modifications, up to 80 modifications, up to 85 modifications, up to 90 modifications, up to 95 modifications, or up to 100 modifications as compared to a CoV S polypeptide having the amino acid sequence of SEQ ID NO: 1 or SEQ ID NO: 2. In aspects, a SARS-COV-2 variant may have between about 2 and about 35 modifications, between about 5 and about 10 modifications, between about 5 and about 20 modifications, between about 10 and about 20 modifications, between about 15 and about 25 modifications, between about 20 and 30 modifications, between about 20 and about 40 modifications, between about 25 and about 45 modifications, between about 25 and about 100 modifications, between about 25 and about 45 modifications, between about 35 and about 100 modifications, as compared to a CoV S polypeptide having the amino acid sequence of SEQ ID NO: 1 or SEQ ID NO: 2.

In embodiments, the heterogenous SARS-COV-2 strain is a SARS-COV-2 virus comprising a CoV S polypeptide with at least about 70%, at least about 75%, at least about 80%, at least about 85%, at least about 90%, at least about 95%, at least about 96%, at least about 97%, at least about 98%, or at least about 99% identity to a CoV S polypeptide having the amino acid sequence of SEQ ID NO: 1 or SEQ ID NO: 2. In embodiments, the heterogeneous SARS-CoV-2 strain is a SARS-COV-2 virus comprising a CoV S polypeptide with between about 70% and about 99.9% identity to a CoV S polypeptide having the amino acid sequence of SEQ ID NO: 1 or SEQ ID NO: 2. In embodiments, the heterogeneous SARS-COV-2 strain is a SARS-COV-2 virus comprising a CoV S polypeptide with between about 70% and about 99.5% identity to a CoV S polypeptide having the amino acid sequence of SEQ ID NO: 1 or SEQ ID NO: 2. In embodiments, the heterogenous SARS-COV-2 strain is a SARS-COV-2 virus comprising a CoV S polypeptide with between about 90% and about 99.9% identity to a CoV S polypeptide having the amino acid sequence of SEQ ID NO: 1 or SEQ ID NO: 2. In embodiments, the heterogeneous SARS-COV-2 strain is a SARS-COV-2 virus comprising a CoV S polypeptide with between about 90% and about 99.8% identity to a CoV S polypeptide having the amino acid sequence of SEQ ID NO: 1 or SEQ ID NO: 2. In embodiments, the heterogeneous SARS-COV-2 strain is a SARS-CoV-2 virus comprising a CoV S polypeptide with between about 95% and about 99.9% identity to a CoV S polypeptide having the amino acid sequence of SEQ ID NO: 1 or SEQ ID NO: 2. In embodiments, the heterogeneous SARS-COV-2 strain is a SARS-COV-2 virus comprising a CoV S polypeptide with between about 95% and about 99.8% identity to a CoV S polypeptide having the amino acid sequence of SEQ ID NO: 1 or SEQ ID NO: 2. In embodiments, the heterogeneous SARS-COV-2 strain is a SARS-COV-2 virus comprising a CoV S polypeptide with between about 95% and about 99% identity to a CoV S polypeptide having the amino acid sequence of SEQ ID NO: 1 or SEQ ID NO: 2. In embodiments, the heterogeneous SARS-COV-2 strain has a World Health Organization Label of alpha, beta, gamma, delta, epsilon, eta, iota, kappa, zeta, mu, or omicron. In embodiments, the heterogenous SARS-COV-2 strain has a PANGO lineage selected from the group consisting of B.1.1.529; BA.1, BA.1.1, BA.2, BA.3, BA.4, BA.5, B.1.1.7, B. 1.351, P.1, B.1.617.2, AY, B.1.427, B.1.429, B.1.525, B.1.526, B.1.617.1, B.1.617.3, P.2, B. 1.621, or B.1.621.1. The following document describes the Pango lineage designation and is incorporated by reference herein in its entirety: O'Toole et al. BMC Genomics, 23, 121 (2022).

In embodiments, the heterogeneous SARS-COV-2 strain has a World Health Organization Label of omicron. In embodiments, the heterogeneous SARS-COV-2 strain with a World Health Organization Label of omicron has at least 35 modifications compared to the wild-type SARS-CoV-2 S polypeptide of SEQ ID NO: 2. In embodiments, the heterogenous SARS-COV-2 strain with a World Health Organization Label of omicron has from 35 to 55, from 35 to 65, from 35 to 75, from 35 to 85, from 35 to 95, or from 35 to 105 modifications compared to the wild-type SARS-COV-2 S polypeptide of SEQ ID NO: 2. In embodiments, the modifications are selected from the group consisting of T6I, T6R, A14S, A54V, V70A, T82I, G129D, H133Q, K134E, W139R, E143G, F144L, Q170E, 1197V, L199I, V200E, V200G, G239V, G244S, G326D, G326H, R333T, L355I, S358F, S358L, S360P, S362F, T363A, D392N, R395S, K404N, N427K, K43IT, V432P, G433S, L439R, L439Q, N447K, S464N, T465K, E471A, F473V, F473S, F477S, Q480R, G483S, Q485R, N488Y, Y492H, T534K, T5911, D601G, G626V, H642Y, N645S, N666K, P668H, S691L, N751K, D783Y, N843K, Q941H, N956K, L968F, D1186N, deletion of amino acid 11, deletion of amino acid 12, deletion of amino acid 13, deletion of amino acid 56, deletion of amino acid 57, deletion of amino acid 130, deletion of amino acid 131, deletion of amino acid 132, deletion of amino acid 144, deletion of amino acid 145, deletion of amino acid 198, and insertion of a tripeptide having the amino acid sequence of EPE between amino acids 214 and 215, and combinations thereof.

In embodiments, the CoV S polypeptide of the variant comprises a combination of modifications selected from the group consisting of:
(i) A54V, T82I, G129D, L199I, G326D, S358L, S360P, S362F, K404N, N427K, G433S, S464N, T465K, E471A, Q480R, G483S, Q485R, N488Y, Y492H, T534K, D601G, H642Y, N666K, P668H, N751K, D783Y, N843K, Q941H, N956K, L968F, deletion of amino acid 56, deletion of amino acid 57, deletion of amino acid 130, deletion of amino acid 131, deletion of amino acid 132, deletion of amino acid 198, and insertion of a tripeptide having the amino acid sequence of EPE between amino acids 214 and 215;
(ii) T6I, A14S, G129D, V200G, G326D, S358F, S360P, S362F, T363A, D392N, R395S, K404N, N427K, S464N, T465K, E471A, Q480R, Q485R, N488Y, Y492H, D601G, H642Y, N666K, P668H, N751K, D783Y, Q941H, N956K, deletion of amino acid 11, deletion of amino acid 12, and deletion of amino acid 13;
(iii) T6R, A14S, T82I, G129D, E143G, L199I, G326D, S358L, S360P, K404N, N427K, G433S, S464N, T465K, E471A, Q480R, G483S, Q485R, N488Y, Y492H, T534K, D601G, H642Y, N666K, P668H, N751K, D783Y, N843K, Q941H, N956K, L968F, deletion of amino acid 144, deletion of amino acid 145, deletion of amino acid 198, and insertion of a tripeptide having the amino acid sequence of EPE between amino acids 214 and 215;
(iv) T6I, A14S, G129D, V200G, G326D, S358F, S360P, S362F, T363A, D392N, K404N, N427K, L439Q, S464N, T465K, E471A, Q480R, Q485R, N488Y, Y492H, D601G, H642Y, N666K, P668H, S691L, N751K, D783Y, Q941H, N956K, deletion of amino acid 11, deletion of amino acid 12, and deletion of amino acid 13;
(v) T6I, A14S, G129D, V200G, G326D, S358F, S360P, S362F, T363A, D392N, S464N, T465K, E471A, Q480R, Q485R, N488Y, Y492H, D601G, H642Y, N666K, P668H, D783Y, Q941H, N956K, deletion of amino acid 11, deletion of amino acid 12, and deletion of amino acid 13;
(vi) T6I, A14S, G129D, V200G, G326D, S358F, S360P, S362F, T363A, D392N, R395S, K404N, D601G, H642Y, N645S, N666K, N751K, D783Y, Q941H, N956K, deletion of amino acid 11, deletion of amino acid 12, deletion of amino acid 13, deletion of amino acid 56, and deletion of amino acid 57;
(vii) V3G, T6I, A14S, G129D, V200G, G326D, S358F, S360P, S362F, T363A, D392N, R395S, K404N, L439R, S464N, T465K, E471A, F473V, Q485R, N488Y, Y492H, D601G, G626V, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(viii) V3G, T6I, A14S, G129D, V200G, G326D, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(ix) T6I, A14S, G129D, V200G, G326D, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(x) T6I, A14S, G129D, K134E, W139R, F144L, 1197V, V200G, G244S, G326H, S358F, S360P, S362F, T363A, D392N, R395S, K404N, N427K, G433S, N447K, S464N, T465K, E471A, Q485R, N488Y, Y492H, D601G, H642Y, N666K, P668H, N751K, D783Y, Q941H, N956K, deletion of amino acid 11, deletion of amino acid 12, and deletion of amino acid 13;
(xi) T6I, A14S, G129D, K134E, W139R, F144L, 1197V, V200G, G244S, G326H, R333T, S358F, S360P, S362F, T363A, D392N, R395S, K404N, N427K, G433S, L439R, N447K, S464N, T465K, E471A, F473S, Q485R, N488Y, Y492H, T5911, D601G, H642Y, N666K, P668H, N751K, D783Y, Q941H, N956K, D1186N, deletion of amino acid 11, deletion of amino acid 12, and deletion of amino acid 13;
(xii) T6I, A14S, G129D, V200G, G326D, R333T, S358F, S360P, S362F, T363A, D392N, R395S, K404N, N427K, L439R, S464N, T465K, E471A, F473V, Q485R, N488Y, Y492H, D601G, H642Y, N645S, N666K, P668H, N751K, D783Y, Q941H, N956K, deletion of amino acid 11, deletion of amino acid 12, deletion of amino acid 13, deletion of amino acid 56, and deletion of amino acid 57;
(xiii) T6I, A14S, G129D, V200G, G326D, R333T, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(xiv) T6I, A14S, V70A, G129D, H133Q, Q170E, V200E, G239V, G326H, R333T, L3551, S358F, S360P, S362F, T363A, D392N, R395S, K404N, N427K, V432P, G433S, N447K, S464N, T465K, E471A, F473S, F477S, Q485R, N488Y, Y492H, D601G, H642Y, N666K, P668H, N751K, D783Y, Q941H, N956K, deletion of amino acid 11, deletion of amino acid 12, deletion of amino acid 13, and deletion of amino acid 131;
(xv) T6I, A14S, G129D, H133Q, Q170E, V200E, G326H, R333T, L355I, S358F, S360P, S362F, T363A, D392N, R395S, K404N, N427K, V432P, G433S, N447K, S464N, T465K, E471A, F473S, F477S, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, deletion of amino acid 57, and deletion of amino acid 131;
(xvi) T6I, A14S, G129D, V200G, G326D, R333T, S358F, S360P, S362F, T363A, D392N, R395S, K404N, N427K, K431T, L439R, N447K, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(xvii) T6I, A14S, G129D, V200G, G326D, S358F, S360P, S362F, T363A, D392N, R395S, K404N, N427K, K431T, L439R, N447K, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57; and
(xviii) T6I, A14S, G129D, V200G, G326D, R333T, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, deletion of amino acid 57, and deletion of amino acid 131;
(xix) deletion of amino acid 56, deletion of amino acid 57, and deletion of amino acid 131, N488Y, A557D, D601G, P668H or P668R, T7031, S969A, and D1105H;
(xx) D67A, K404N, E471K, N488Y, D601G, and A688V;
(xxi) D67A, D202G, L229H, K404N, E471K, N488Y, D601G, and A688V;
(xxii) D67A, D202G, deletion of 1, 2, or 3 amino acids of amino acids 228-230, K404N, E471K, N488Y, D601G, and A688V;
(xxiii) D67A, L229H, R2331, N488Y, K404N, E471K, D601G, and A688V;
(xxiv) L5F, T7N, P13S, D125Y, R177S, K404T, E471K, N488Y, D601G, H642Y, T1014I, and V1163F;
(xxv) W139C and L439;
(xxvi) deletion of amino acid 144, deletion of amino acid 145, T6R, E143G, L439R, T465K, D601G, P668R, and D937N;
(xxvii) deletion of amino acid 144, deletion of amino acid 145, T6R, G129D, E143G, L439R, T465K, D601G, P668R, and D937N;
(xxviii) deletion of amino acid 144, deletion of amino acid 145, T6R, T82I, G129D, Y132H, E143G, A209V, K404N L439R, T465K, D601G, P668R, and D937N;
(xxix) deletion of amino acid 144, deletion of amino acid 145, T6R, G129D, E143G, W245I, K404N, N426K, L439R, T465K, E471K, N488Y, D601G, P668R, and D937N;
(xxx) deletion of amino acid 144, deletion of amino acid 145, T6R, W51H, H53W, G129D, E143G, D200V, L201R, W245I, K404N, N426K, L439R, T465K, E471K, N488Y, D601G, P668R, and D937N;
(xxxi) deletion of amino acid 144, deletion of amino acid 145, T6R, G129D, E143G, K404N, L439R, T465K, E471Q, D601G, P668R, and D937N;
(xxxii) Q39R, A54V, E471K; D601G, Q664H, F875L, and deletion of 1, 2, 3, or 4 of amino acids 56, 57, 131, 132;
(xxxiii) T82I, D240G, E471K, D601G, and A688V;
(xxxiv) L439R, E471Q, D601G, P668R, and Q1058H;
(xxxv) G62V, T63I, R233N, L439Q, F477S, D601G, T846N, and deletion of 1, 2, 3, 4, 5, or 6 of amino acids 234-240;
(xxxvi) T82I, Y131S, Y132N, R333K, E471K, N488Y, D601G, P668H, and D937N; and
(xxxvii) G129D, G326D, S360P, S362F, K404N, N427K, T465K, E471A or E471K, Q480K or Q480R, Q485R, N488Y, Y492H, D601G, H642Y, N666K, P668H, N751K, D783Y, Q941H, and N953K;
(xxxviii) F456L, T572I, F59S, R346T, A1087S or A475V;
wherein the amino acids of the CoV S glycoprotein are numbered with respect to a polypeptide having the sequence of SEQ ID NO: 1 or SEQ ID NO: 2.

The term “efficacy” of an immunogenic composition or vaccine composition described herein refers to the percentage reduction of disease (e.g., COVID-19) in a group administered an immunogenic composition as compared to a group that is not administered the immunogenic composition. In embodiments, efficacy (E) is calculated using the following equation: E (%)=(1−RR)×100, where RR=relative risk of incidence rates between the group administered the immunogenic composition and the group that is not administered the immunogenic composition. In embodiments, immunogenic compositions described herein have an efficacy against a SARS-CoV-2 virus or heterogeneous SARS-COV-2 strain(s) that is at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, between about 50% and about 99%, between about 50% and about 98%, between about 60% and about 99%, between about 60% and about 98%, between about 70% and about 98%, between about 70% and about 95%, between about 70% and about 99%, between about 80% and about 99%, between about 80% and about 98%, between about 80% and about 95%, between about 85% and about 99%, between about 85% and about 98%, between about 85% and about 95%, between about 90% and about 95%, between about 90% and 98%, or between about 90% and about 99%.

### Compositions Containing Coronavirus (CoV) Spike(S) Proteins

The disclosure provides coronavirus (CoV) Spike(S) polypeptides, including naturally and non-naturally occurring polypeptides, nanoparticles containing CoV S polypeptides, and immunogenic compositions and vaccine compositions containing either CoV S polypeptides or nanoparticles containing CoV S polypeptides. In embodiments, provided herein are methods of using CoV S polypeptides, nanoparticles, immunogenic compositions, and vaccine compositions to stimulate an immune response against a SARS-COV-2 virus or a heterogeneous SARS-COV-2 strain. In embodiments, the heterogeneous SARS-COV-2 strain has a PANGO lineage selected from the group consisting of B.1.1.529; BA.1, BA.1.1, BA.2, BA.3, BA.4, BA.5, B.1.1.7, B.1.351, P.1, B.1.617.2, AY, B.1.427, B.1.429, B.1.525, B.1.526, B.1.617.1, B.1.617.3, P.2, B.1.621, or B.1.621.1. In embodiments, the heterogeneous SARS-COV-2 strain has a World Health Organization label of alpha, beta, gamma, delta, epsilon, eta, iota, kappa, zeta, mu, or omicron.

Also provided herein are methods of manufacturing the nanoparticles and vaccine compositions. Advantageously, the methods provide nanoparticles that are substantially free from contamination by other proteins, such as proteins associated with recombinant expression of proteins in insect cells. In embodiments, expression occurs in baculovirus/Sf9 or baculovirus/Sf22a systems.

### CoV S Polypeptide Antigens

The immunogenic compositions of the disclosure contain from about 1 to about 15, from about 2 to about 15, from about 3 to about 15, from about 3 to about 12, from about 4 to about 6, from about 3 to about 7, from about 4 to about 12, from about 5 to about 8, from about 6 to about 9, or from about 5 to 10 non-naturally occurring CoV S polypeptides. In embodiments, the immunogenic compositions comprise 1, 2, 3, 4, 5, 6, 7, 8, 9, 10, 11, 12, 13, 14, 15, 16, 17, 18, 19, 20, 21, 22, 23, 24, or 25 different CoV S polypeptides. In embodiments, the immunogenic compositions comprise five non-naturally occurring CoV S polypeptides. The CoV S polypeptides may be derived from coronaviruses, including but not limited to SARS-COV-2, for example from SARS-COV-2, from MERS CoV, and from SARS COV.

In embodiments, the CoV S polypeptides are derived from SARS-COV-2. In embodiments, the CoV S polypeptide is derived from a heterogeneous SARS-COV-2 strain. The SARS-COV-2 S protein has a four amino acid insertion in the S1/S2 cleavage site resulting in a polybasic RRAR furin-like cleavage motif. The SARS-COV-2 S protein is synthesized as an inactive precursor (SO) that is proteolytically cleaved at the furin cleavage site into S1 and S2 subunits which remain non-covalently linked to form prefusion trimers. The S2 domain of the SARS-COV-2 S protein comprises a fusion peptide (FP), two heptad repeats (HR1 and HR2), a transmembrane (TM) domain, and a cytoplasmic tail (CT). The S1 domain of the SARS-COV-2 S protein folds into four distinct domains: the N-terminal domain (NTD) and the C-terminal domain, which contains the receptor binding domain (RBD) and two subdomains SD1 and SD2. The prefusion SARS-COV-2 S protein trimers undergo a structural rearrangement from a prefusion to a postfusion conformation upon S-protein receptor binding and cleavage.

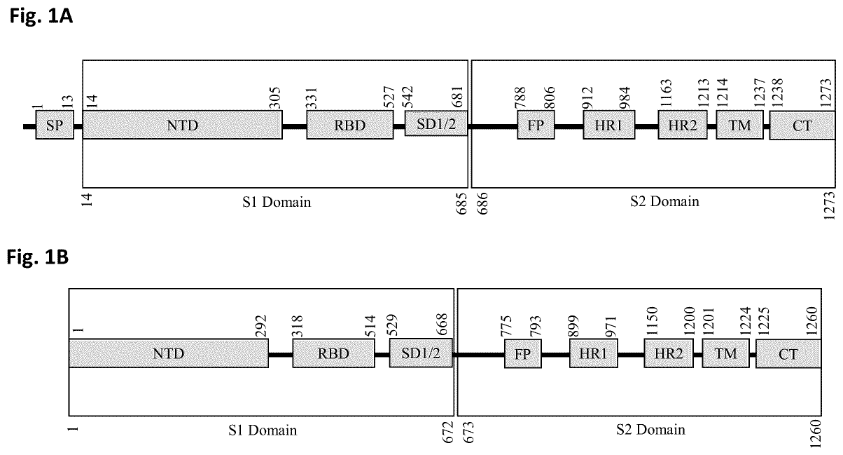

In embodiments, the CoV S polypeptides are glycoproteins, due to post-translational glycosylation. The glycoproteins comprise one or more domains, including a signal peptide, an S1 subunit, an S2 subunit, a NTD, a, RBD, two subdomains (SD1 and SD2, labeled SD1/2 in FIGS. 1A-B and referred to as “SD1/2” herein), an intact or modified fusion peptide, an HR1 domain, an HR2 domain, a TM, and a CD. In embodiments, the amino acids for each domain are given in FIG. 1A (shown according to SEQ ID NO: 1), FIG. 1B (shown according to SEQ ID NO: 2).

In embodiments, the immunogenic composition comprises (i) a first CoV S polypeptide from SARS-COV-2 or from a heterogeneous SARS-COV-2 strain, wherein the first CoV S polypeptide contains an inactive furin cleavage site and mutations of amino acids 973 and 974, wherein the CoV S polypeptide is numbered according to SEQ ID NO: 2, and (ii) a second CoV S polypeptide with from about 1 to about 50 modifications compared to the first CoV S polypeptide. In embodiment, the immunogenic composition comprises a third, fourth, fifth, sixth, seventh, eight, ninth, or tenth CoV S polypeptide, wherein each CoV S polypeptide contains from about 1 to about 50 modifications compared to the first CoV S polypeptide.

In embodiments, the amino acid sequence of an S1 subunit, S2 subunit, NTD, RBD, SD1/2, fusion peptide, HR1 domain, HR2 domain, TM domain, or CD of the second, third, fourth, fifth, sixth, seventh, eight, ninth, or tenth CoV S polypeptide is independently at least 90%, at least 91%, at least 92%, at least 93%, at least 94%, at least 95%, at least 96%, at least 97%, at least 98%, at least 99%, or at least 99.5% identical to the amino acid sequence of the respective domain of the first polypeptide.

In embodiments, the amino acid sequence of an S1 subunit, S2 subunit, NTD, RBD, SD1/2, fusion peptide, HR1 domain, HR2 domain, TM domain, or CD of the second, third, fourth, fifth, sixth, seventh, eight, ninth, or tenth CoV S polypeptide contains up to about 1, up to about 2, up to about 3, up to about 4, up to about 5, up to about 6, up to about 7, up to about 8, up to about 9, up to about 10, up to about 15, up to about 20, up to about 25, up to about 30, from 1 to about 5, from about 2 to about 4, from about 3 to about 5, from about 3 to about 10, from about 5 to about 10, from about 6 to about 12, from about 8 to about 12, from about 10 to about 15, from about 15 to about 20, from about 12 to about 18, or from about 18 to about 25 modifications compared to the amino acid sequence of the respective domain of the first polypeptide.

In embodiments, the amino acid sequence of the second, third, fourth, fifth, sixth, seventh, eighth, nine, or tenth CoV S polypeptide is at least 90%, at least 91%, at least 92%, at least 93%, at least 94%, at least 95%, at least 96%, at least 97%, at least 98%, at least 99% identical to the amino acid sequence of the first CoV S polypeptide, but no more than 99.92% identical to the amino acid sequence of the first polypeptide.

In embodiments, the compositions described herein may be used to stimulate immune responses against SARS-COV-2 or a heterogeneous SARS-COV-2 strain.

In embodiments, the CoV S polypeptides described herein exist in a prefusion conformation. In embodiments, the CoV S polypeptides described herein comprise a flexible HR2 domain. Unless otherwise mentioned, the flexibility of a domain is determined by transition electron microscopy (TEM) and 2D class averaging. A reduction in electron density corresponds to a flexible domain.

CoV S Polypeptide Antigens-Modifications to S1 subunit

In embodiments, the CoV S polypeptides contain one or more modifications to the S1 subunit having an amino acid sequence of SEQ ID NO: 121.

The amino acid sequence of the S1 subunit (SEQ ID NO: 121) is shown below.

QCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLKP
FERDISTEIYQAGSTPCNGVEGFNCYFPLQSYGFQPTNGVGYQPYRVVVL
SFELLHAPATVCGPKKSTNLVKNKCVNFNFNGLTGTGVLTESNKKFLPFQ
QFGRDIADTTDAVRDPQTLEILDITPCSFGGVSVITPGTNTSNQVAVLYQ
DVNCTEVPVAIHADQLTPTWRVYSTGSNVFQTRAGCLIGAEHVNNSYECD
IPIGAGICASYQTQTNSPRRAR

In embodiments, the CoV S polypeptides described herein comprise an S1 subunit with at least 90%, at least 91%, at least 92%, at least 93%, at least 94%, at least 95%, at least 96%, at least 97%, at least 98%, at least 99%, or at least 99.5%, identity to the S1 subunit of SEQ ID NO: 1 or SEQ ID NO: 2. The S1 subunit may have a deletion, an insertion, or mutation of up to about 1, up to about 2, up to about 3, up to about 4, up to about 5, up to about 10, up to about 15, up to about 20, up to about 25, up to about 30 amino acids, up to about 35 amino acids, up to about 40 amino acids, up to about 45 amino acids, or up to about 50 amino acids compared to the amino acid sequence of the S1 subunit of SEQ ID NO: 1 or SEQ ID NO: 2. The S1 subunit may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the S1 subunit of SEQ ID NO: 1 or SEQ ID NO: 2.

In embodiments, the S1 subunit may contain any combination of modifications shown in Table 1A.

TABLE 1A
Modifications to S1 (SEQ ID NO: 121)
* amino acids 14-685 of SEQ ID NO: 1 and amino
acids 1-672 of SEQ ID NO: 2

| Position | Position | Position | |
|---|---|---|---|
| within | within | within | |
| SEQ ID | SEQ ID | SEQ ID | |
| NO: 1 | NO: 2 | NO: 121 | Potential Modifications |
| 14-305 | 1-292 | 1-292 | deletion of up to about 1, 2, 3, 4, 5, |
| | | | 10, 20, 30, 40, 50, 60, 70, 80, 90, |
| | | | 100, 110, 120, 130, 140, 150, 160, 170, |
| | | | 180, 190, 200, 210, 220, 230, 240, 250, |
| | | | 260, 270, 280, 290, or 292 amino acids |
| 18 | 5 | 5 | mutation to phenylalanine |
| | | | mutation to tyrosine |
| | | | mutation to tryptophan |
| 19 | 6 | 6 | mutation to arginine |
| | | | mutation to lysine |
| | | | mutation to histidine |
| | | | mutation to isoleucine |
| 20 | 7 | 7 | mutation to asparagine |
| | | | mutation to glutamine |
| | | | mutation to isoleucine |
| | | | mutation to valine |
| 24 | 11 | 11 | mutation to serine |
| | | | mutation to threonine |
| | | | deletion |
| 25 | 12 | 12 | insertion of amino acids |
| | | | proline-proline-alanine |
| | | | (PPA) after amino acid 25 |
| | | | deletion |
| 26 | 13 | 13 | mutation to serine |
| | | | mutation to threonine |
| | | | deletion |
| 27 | 14 | 14 | mutation to serine |
| | | | mutation to threonine |
| 52 | 39 | 39 | mutation to arginine |
| | | | mutation to lysine |
| | | | mutation to histidine |
| 64 | 51 | 51 | mutation to histidine |
| | | | mutation to lysine |
| | | | mutation to arginine |
| 66 | 53 | 53 | mutation to tryptophan |
| | | | mutation to tyrosine |
| | | | mutation to phenylalanine |
| 67 | 54 | 54 | mutation to valine |
| | | | mutation to isoleucine |
| | | | mutation to leucine |
| 69 | 56 | 56 | Deletion of amino acid |
| 70 | 57 | 57 | Deletion of amino acid |
| | | | Mutation to phenylalanine |
| | | | Mutation to tyrosine |
| | | | Mutation to tryptophan |
| 75 | 62 | 62 | Mutation to valine |
| | | | Mutation to leucine |
| | | | Mutation to isoleucine |
| 76 | 63 | 63 | Mutation to isoleucine |
| | | | Mutation to valine |
| | | | Mutation to leucine |
| 80 | 67 | 67 | mutation to alanine |
| | | | mutation to glycine |
| 83 | 70 | 70 | mutation to alanine |
| 95 | 82 | 82 | mutation to beta branched amino acid |
| | | | mutation to isoleucine |
| | | | mutation to valine |
| 138 | 125 | 125 | mutation to tyrosine |
| | | | mutation to phenylalanine |
| | | | mutation to tryptophan |
| 142 | 129 | 129 | mutation to aspartic acid |
| | | | mutation to glutamic acid |
| 143 | 130 | 130 | deletion of amino acid |
| 144 | 131 | 131 | Deletion of amino acid |
| | | | Mutation to serine |
| 145 | 132 | 132 | Deletion of amino acid |
| | | | Mutation to histidine |
| | | | Mutation to asparagine |
| | | | Mutation to glutamine |
| 146 | 133 | 133 | mutation to aromatic amino acid |
| | | | mutation to tyrosine |
| | | | mutation to phenylalanine |
| | | | mutation to tryptophan |
| | | | mutation to glutamine |
| 147 | 134 | 134 | mutation to glutamic acid |
| | | | mutation to aspartic acid |
| 152 | 139 | 139 | mutation to cysteine |
| | | | mutation to methionine |
| | | | mutation to serine |
| | | | mutation to threonine |
| | | | mutation to arginine |
| | | | mutation to lysine |
| 156 | 143 | 143 | mutation to glycine |
| | | | mutation to alanine |
| 157 | 144 | 144 | deletion of amino acid |
| | | | mutation to leucine |
| | | | mutation to isoleucine |
| | | | mutation to valine |
| | | | mutation to beta-branched amino acid |
| 158 | 145 | 145 | deletion of amino acid |
| 180 | 167 | 167 | mutation to beta-branched amino acid |
| | | | mutation to valine |
| | | | mutation to isoleucine |
| | | | mutation to leucine |
| 183 | 170 | 170 | mutation to glutamic acid |
| | | | mutation to aspartic acid |
| 190 | 177 | 177 | mutation to serine |
| | | | mutation to threonine |
| | | | mutation to cysteine |
| 210 | 197 | 197 | mutation to valine |
| | | | mutation to isoleucine |
| | | | mutation to leucine |
| | | | mutation to beta branched amino acid |
| 211 | 198 | 198 | mutation to isoleucine |
| | | | mutation to valine |
| | | | mutation to leucine |
| | | | mutation to beta branched amino acid |
| | | | deletion of amino acid |
| 212 | 199 | 199 | mutation to isoleucine |
| | | | mutation to valine |
| | | | mutation to leucine |
| | | | mutation to beta branched amino acid |
| | | | deletion of amino acid |
| 213 | 200 | 200 | mutation to valine |
| | | | mutation to leucine |
| | | | mutation to isoleucine |
| | | | mutation to beta branched amino acid |
| | | | mutation to proline |
| | | | mutation to glycine |
| | | | mutation to glutamic acid |
| | | | mutation to aspartic acid |
| 214 | 201 | 201 | mutation to arginine |
| | | | mutation to lysine |
| | | | mutation to histidine |
| | | | mutation to aspartic acid |
| | | | mutation to glutamic acid |
| | | | insertion of amino acids |
| | | | glutamic acid-proline- |
| | | | glutamic acid (EPE) after 214 |
| 215 | 202 | 202 | mutation to glycine |
| | | | mutation to alanine |
| | | | insertion of amino acids |
| | | | glutamic acid-proline- |
| | | | glutamic acid (EPE) after 215 |
| 222 | 209 | 209 | mutation to valine |
| | | | mutation to leucine |
| | | | mutation to isoleucine |
| 241-244 | 228-231 | 228-231 | deletion of 1, 2, 3, or 4 amino acids |
| 242 | 229 | 229 | mutation to histidine |
| | | | mutation to lysine |
| | | | mutation to arginine |
| 246 | 233 | 233 | mutation to beta-branched amino acid |
| | | | mutation to isoleucine |
| | | | mutation to valine |
| | | | mutation to threonine |
| | | | mutation to asparagine |
| 247 | 234 | 234 | deletion of amino acid |
| 248 | 235 | 235 | deletion of amino acid |
| 249 | 236 | 236 | deletion of amino acid |
| 250 | 237 | 237 | deletion of amino acid |
| 251 | 238 | 238 | deletion of amino acid |
| 252 | 239 | 239 | deletion of amino acid |
| | | | mutation to valine |
| | | | mutation to leucine |
| | | | mutation to isoleucine |
| | | | mutation to beta branched amino acid |
| | | | mutation to glycine |
| 253 | 240 | 240 | mutation to glycine |
| | | | deletion of amino acid |
| 257 | 244 | 244 | mutation to serine |
| | | | mutation to threonine |
| | | | mutation to asparagine |
| | | | mutation to glutamine |
| 258 | 245 | 245 | mutation to isoleucine |
| | | | mutation to valine |
| | | | mutation to leucine |
| | | | mutation to beta branched amino acid |
| 339 | 326 | 326 | mutation to aspartic acid |
| | | | mutation to glutamic acid |
| | | | mutation to histidine |
| 346 | 333 | 333 | mutation to lysine |
| | | | mutation to arginine |
| | | | mutation to histidine |
| | | | mutation to threonine |
| | | | mutation to serine |
| 368 | 355 | 355 | mutation to isoleucine |
| | | | mutation to leucine |
| | | | mutation to valine |
| | | | mutation to beta-branched amino acid |
| 371 | 358 | 358 | mutation to leucine |
| | | | mutation to isoleucine |
| | | | mutation to valine |
| | | | mutation to phenylalanine |
| 373 | 360 | 360 | mutation to proline |
| 375 | 362 | 362 | mutation to phenylalanine |
| | | | mutation to tyrosine |
| | | | mutation to tryptophan |
| 376 | 363 | 363 | mutation to alanine |
| | | | mutation to glycine |
| 405 | 392 | 392 | mutation to asparagine |
| | | | mutation to glutamine |
| 408 | 395 | 395 | mutation to serine |
| | | | mutation to threonine |
| 417 | 404 | 404 | mutation to asparagine |
| | | | mutation to threonine |
| | | | mutation to isoleucine |
| | | | mutation to valine |
| | | | mutation to serine |
| | | | mutation to glutamine |
| | | | mutation to beta-branched amino acid |
| 432 | 419 | 419 | mutation to lysine |
| | | | mutation to arginine |
| | | | mutation to histidine |
| 439 | 426 | 426 | mutation to lysine |
| | | | mutation to arginine |
| | | | mutation to histidine |
| 440 | 427 | 427 | mutation to lysine |
| | | | mutation to arginine |
| | | | mutation to histidine |
| 444 | 431 | 431 | mutation to threonine |
| | | | mutation to serine |
| 445 | 432 | 432 | mutation to proline |
| 446 | 433 | 433 | mutation to serine |
| | | | mutation to threonine |
| | | | mutation to asparagine |
| | | | mutation to glutamine |
| 452 | 439 | 439 | mutation to arginine |
| | | | mutation to lysine |
| | | | mutation to histidine |
| | | | mutation to glutamine |
| | | | mutation to asparagine |
| 453 | 440 | 440 | mutation to phenylalanine |
| | | | mutation to tryptophan |
| 456 | 443 | 443 | mutation to leucine |
| | | | mutation to valine |
| | | | mutation to isoleucine |
| 460 | 447 | 447 | mutation to lysine |
| | | | mutation to arginine |
| 477 | 464 | 464 | mutation to asparagine |
| | | | mutation to glutamine |
| 478 | 465 | 465 | mutation to lysine |
| | | | mutation to arginine |
| | | | mutation to histidine |
| 484 | 471 | 471 | mutation to alanine |
| | | | mutation to lysine |
| | | | mutation to arginine |
| | | | mutation to histidine |
| | | | mutation to glutamine |
| | | | mutation to asparagine |
| 486 | 473 | 473 | mutation to valine |
| | | | mutation to leucine |
| | | | mutation to isoleucine |
| | | | mutation to serine |
| | | | mutation to threonine |
| | | | mutation to proline |
| 490 | 477 | 477 | mutation to serine |
| | | | mutation to threonine |
| 493 | 480 | 480 | mutation to lysine |
| | | | mutation to arginine |
| | | | mutation to histidine |
| 494 | 481 | 481 | mutation to proline |
| 496 | 483 | 483 | mutation to serine |
| | | | mutation to threonine |
| 498 | 485 | 485 | mutation to lysine |
| | | | mutation to arginine |
| | | | mutation to histidine |
| 501 | 488 | 488 | mutation to tyrosine |
| | | | mutation to phenylalanine |
| | | | mutation to tryptophan |
| 505 | 492 | 492 | mutation to histidine |
| 521 | 508 | 508 | mutation to serine |
| | | | mutation to threonine |
| 547 | 534 | 534 | mutation to lysine |
| | | | mutation to arginine |
| | | | mutation to histidine |
| 570 | 557 | 557 | mutation to aspartic acid |
| | | | mutation to glutamic acid |
| 604 | 591 | 591 | mutation to isoleucine |
| | | | mutation to leucine |
| | | | mutation to valine |
| | | | mutation to beta branched amino acid |
| 613 | 600 | 600 | mutation to histidine |
| | | | mutation to lysine |
| | | | mutation to arginine |
| 614 | 601 | 601 | mutation to glycine |
| | | | mutation to alanine |
| 639 | 626 | 626 | mutation to valine |
| | | | mutation to leucine |
| | | | mutation to isoleucine |
| 655 | 642 | 642 | mutation to tyrosine |
| | | | mutation to phenylalanine |
| | | | mutation to tryptophan |
| 658 | 645 | 645 | mutation to serine |
| | | | mutation to threonine |
| 677 | 664 | 664 | mutation to histidine |
| 679 | 666 | 666 | mutation to lysine |
| | | | mutation to arginine |
| | | | mutation to histidine |
| 681 | 668 | 668 | mutation to histidine |
| | | | mutation to lysine |
| | | | mutation to arginine |
| 682-685 | 669-672 | 669-672 | inactive furin cleavage |
| | | | site (See Table 1E) |

CoV S Polypeptide Antigens-Modifications to S1 subunit-NTD

In embodiments, the CoV S polypeptides contain one or more modifications to the NTD. In embodiments, the NTD has an amino acid sequence of SEQ ID NO: 118, which corresponds to amino acids 14-305 of SEQ ID NO: 1 or amino acids 1-292 of SEQ ID NO: 2.

The amino acid sequence of an NTD (SEQ ID NO: 118) is shown below.

QCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAA
AYYVGYLQPRTFLLKYNENGTITDAVDCALDPLSETKCTLKS

In embodiments, the NTD has an amino acid sequence of SEQ ID NO: 45, which corresponds to amino acids 14 to 331 of SEQ ID NO: 1 or amino acids 1-318 of SEQ ID NO: 2. The amino acid sequence of an NTD (SEQ ID NO: 45) is shown below.

QCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIY
QTSNFRVQPTESIVRFPN

In embodiments, the NTD and RBD overlap by up to about 1 amino acid, up to about 5 amino acids, up to about 10 amino acids, or up to about 20 amino acids.

In embodiments, an NTD as provided herein may be extended at the C-terminus by up to 5, up to 10, up to 15, up to 20, up to 25, or up to 30 amino acids.

In embodiments, the CoV S polypeptides described herein comprise a NTD with at least 95%, at least 96%, at least 97%, at least 98%, at least 99%, or at least 99.5%, identity to the NTD of SEQ ID NO: 1 or SEQ ID NO: 2. The NTD may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the NTD of SEQ ID NO: 1 or SEQ ID NO: 2. The NTD may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the NTD of SEQ ID NO: 1 or SEQ ID NO: 2.

In embodiments, the CoV S polypeptides contain a deletion of one or more amino acids from the N-terminal domain (NTD) (corresponding to amino acids 1-292 of SEQ ID NO: 2. In embodiments, the CoV S polypeptides contain a deletion of up to about 10, 20, 30, 40, 50, 60, 70, 80, 90, 100, 110, 120, 130, 140, 150, 160, 170, 180, 190, 200, 210, 220, 230, 240, 250, 260, 270, 280, 290, or 292 amino acids of the NTD.

In embodiments, the CoV S polypeptides contain a deletion of one or more amino acids from the NTD (corresponding to amino acids 1-318 of SEQ ID NO: 2). In embodiments, the CoV S polypeptides contain a deletion of amino acids 1-318 of the NTD of SEQ ID NO: 2. In embodiments, deletion of the NTD enhances protein expression of the CoV Spike(S) polypeptide. In embodiments, the CoV S polypeptides which have an NTD deletion have amino acid sequences represented by SEQ ID NOS: 46, 48, 49, 51, 52, and 54. In embodiments, the CoV S polypeptides which have an NTD deletion are encoded by an isolated nucleic acid sequence selected from the group consisting of SEQ ID NO: 47, SEQ ID NO: 50, and SEQ ID NO: 53.

In embodiments, the NTD may contain any combination of modifications shown in Table 1B. The modifications are shown with respect to SEQ ID NO: 2, the mature S polypeptide sequence for reference.

TABLE 1B
Modifications to NTD (SEQ ID NO: 118)

| Position within | SEQ ID NO: 2 | SEQ ID NO: 118 or | |
|---|---|---|---|
| SEQ ID NO: 1 | residue | SEQ ID NO: 45 residue | Modifications |
| 14-305 | 1-292 | 1-292 | deletion of up to about 1, 2, 3, 4, 5, 10, 20, 30, 40, |
| | | | 50, 60, 70, 80, 90, 100, 110, 120, 130, 140, 150, |
| | | | 160, 170, 180, 190, 200, 210, 220, 230, 240, 250, |
| | | | 260, 270, 280, 290, or 292 amino acids |
| 18 | 5 | 5 | mutation to phenylalanine |
| | | | mutation to tyrosine |
| | | | mutation to tryptophan |
| 19 | 6 | 6 | mutation to arginine |
| | | | mutation to lysine |
| | | | mutation to histidine |
| | | | mutation to isoleucine |
| 20 | 7 | 7 | mutation to asparagine |
| | | | mutation to glutamine |
| | | | mutation to isoleucine |
| | | | mutation to valine |
| 24 | 11 | 11 | mutation to serine |
| | | | mutation to threonine |
| | | | deletion |
| 25 | 12 | 12 | insertion of amino acids proline-proline-alanine |
| | | | (PPA) after amino acid 25 |
| | | | deletion |
| 26 | 13 | 13 | mutation to serine |
| | | | mutation to threonine |
| | | | deletion |
| 27 | 14 | 14 | mutation to serine |
| | | | mutation to threonine |
| 52 | 39 | 39 | mutation to arginine |
| | | | mutation to lysine |
| | | | mutation to histidine |
| 64 | 51 | 51 | mutation to histidine |
| | | | mutation to lysine |
| | | | mutation to arginine |
| 66 | 53 | 53 | mutation to tryptophan |
| | | | mutation to tyrosine |
| | | | mutation to phenylalanine |
| 67 | 54 | 54 | mutation to valine |
| | | | mutation to isoleucine |
| | | | mutation to leucine |
| 69 | 56 | 56 | deletion of amino acid |
| 70 | 57 | 57 | deletion of amino acid |
| | | | mutation to phenylalanine |
| | | | mutation to tyrosine |
| | | | mutation to tryptophan |
| 75 | 62 | 62 | mutation to valine |
| | | | mutation to leucine |
| | | | mutation to isoleucine |
| 76 | 63 | 63 | mutation to isoleucine |
| | | | mutation to valine |
| | | | mutation to leucine |
| 80 | 67 | 67 | mutation to alanine |
| | | | mutation to glycine |
| 83 | 70 | 70 | mutation to alanine |
| 95 | 82 | 82 | mutation to beta branched amino acid |
| | | | mutation to isoleucine |
| | | | mutation to valine |
| 138 | 125 | 125 | mutation to tyrosine |
| | | | mutation to phenylalanine |
| | | | mutation to tryptophan |
| 142 | 129 | 129 | mutation to aspartic acid |
| | | | mutation to glutamic acid |
| 143 | 130 | 130 | deletion of amino acid |
| 144 | 131 | 131 | deletion of amino acid |
| | | | mutation to serine |
| 145 | 132 | 132 | deletion of amino acid |
| | | | mutation to histidine |
| | | | mutation to asparagine |
| | | | mutation to glutamine |
| 146 | 133 | 133 | mutation to aromatic amino acid |
| | | | mutation to tyrosine |
| | | | mutation to phenylalanine |
| | | | mutation to tryptophan |
| | | | mutation to glutamine |
| | | | mutation to asparagine |
| 147 | 134 | 134 | mutation to glutamic acid |
| | | | mutation to aspartic acid |
| 152 | 139 | 139 | mutation to cysteine |
| | | | mutation to methionine |
| | | | mutation to serine |
| | | | mutation to threonine |
| | | | mutation to arginine |
| | | | mutation to lysine |
| 156 | 143 | 143 | mutation to glycine |
| | | | mutation to alanine |
| 157 | 144 | 144 | deletion of amino acid |
| | | | mutation to leucine |
| 158 | 145 | 145 | deletion of amino acid |
| 180 | 167 | 167 | mutation to isoleucine |
| | | | mutation to valine |
| | | | mutation to leucine |
| | | | mutation to beta branched amino acid |
| 183 | 170 | 170 | mutation to glutamic acid |
| | | | mutation to aspartic acid |
| 190 | 177 | 177 | mutation to serine |
| | | | mutation to threonine |
| | | | mutation to cysteine |
| 210 | 197 | 197 | mutation to valine |
| | | | mutation to isoleucine |
| | | | mutation to leucine |
| | | | mutation to beta branched amino acid |
| 211 | 198 | 198 | mutation to isoleucine |
| | | | mutation to valine |
| | | | mutation to leucine |
| | | | mutation to beta branched amino acid |
| | | | deletion of amino acid |
| 212 | 199 | 199 | mutation to isoleucine |
| | | | mutation to valine |
| | | | mutation to leucine |
| | | | mutation to beta branched amino acid |
| | | | deletion of amino acid |
| 213 | 200 | 200 | mutation to valine |
| | | | mutation to leucine |
| | | | mutation to isoleucine |
| | | | mutation to beta branched amino acid |
| | | | mutation to proline |
| | | | mutation to glycine |
| | | | mutation to glutamic acid |
| | | | mutation to aspartic acid |
| 214 | 201 | 201 | mutation to arginine |
| | | | mutation to lysine |
| | | | mutation to histidine |
| | | | mutation to aspartic acid |
| | | | mutation to glutamic acid |
| | | | insertion of amino acids glutamic acid-proline- |
| | | | glutamic acid (EPE) after 214 |
| 215 | 202 | 202 | mutation to glycine |
| | | | mutation to alanine |
| | | | insertion of amino acids glutamic acid-proline- |
| | | | glutamic acid (EPE) after 215 |
| 222 | 209 | 209 | mutation to valine |
| | | | mutation to leucine |
| | | | mutation to isoleucine |
| 241-244 | 228-231 | 228-231 | deletion of 1, 2, 3, or 4 amino acids |
| 242 | 229 | 229 | mutation to histidine |
| | | | mutation to lysine |
| | | | mutation to arginine |
| 246 | 233 | 233 | mutation to beta-branched amino acid |
| | | | mutation to isoleucine |
| | | | mutation to valine |
| | | | mutation to threonine |
| | | | mutation to asparagine |
| 247 | 234 | 234 | deletion of amino acid |
| 248 | 235 | 235 | deletion of amino acid |
| 249 | 236 | 236 | deletion of amino acid |
| 250 | 237 | 237 | deletion of amino acid |
| 251 | 238 | 238 | deletion of amino acid |
| 252 | 239 | 239 | deletion of amino acid |
| | | | mutation to valine |
| | | | mutation to isoleucine |
| | | | mutation to leucine |
| | | | mutation to beta branched amino acid |
| | | | mutation to glycine |
| 253 | 240 | 240 | mutation to glycine |
| | | | deletion of amino acid |
| 257 | 244 | 244 | mutation to serine |
| | | | mutation to threonine |
| 258 | 245 | 245 | mutation to isoleucine |
| | | | mutation to valine |
| | | | mutation to leucine |
| | | | mutation to beta branched amino acid |

*amino acids 14-305 of SEQ ID NO: 1 and amino acids 1-292 of SEQ ID NO: 2

### CoV S Polypeptide Antigens-Modifications to S1 Subunit-RBD

In embodiments, the CoV S polypeptides contain one or more modifications to the RBD.

In embodiments, the RBD has an amino acid sequence of SEQ ID NO: 126, which corresponds to amino acids 331-527 of SEQ ID NO: 1 or amino acids 318-514 of SEQ ID NO: 2.

The amino acid sequence of the RBD (SEQ ID NO: 126) is shown below:

NITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGP

In embodiments, the RBD has an amino acid sequence of SEQ ID NO: 116, which corresponds to amino acids 335-530 of SEQ ID NO: 1 or amino acids 322-517 of SEQ ID NO: 2.

The amino acid sequence of the RBD (SEQ ID NO: 116) is shown below.

LCPFGEVFNATRFASVYAWNRKRISNCVADYSVLYNSASFSTFKCYGVSP
TKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVE
GFNCYFPLQSYGFQPTNGVGYQPYRVVVLSFELLHAPATVCGPKKS

In embodiments, an RBD as provided herein may be extended at the N-terminus or C-terminus by up to 1 amino acid, up to 5 amino acids, up to 10 amino acids, up to 15 amino acids, up to 20 amino acids, up to 25 amino acids, or up to 30 amino acids.

In embodiments, the CoV S polypeptides described herein comprise a RBD with at least 95%, at least 96%, at least 97%, at least 98%, at least 99%, or at least 99.5%, identity to the RBD of SEQ ID NO: 1 or SEQ ID NO: 2. The RBD may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the RBD of SEQ ID NO: 1 or SEQ ID NO: 2. The RBD may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the RBD of SEQ ID NO: 1 or SEQ ID NO: 2.

In embodiments, the CoV S polypeptide has at least one, at least two, at least three, at least four, at least four, at least 5, at least 6, at least 7, at least 8, at least 9, at least 10, at least 11, at least 12, at least 13, at least 14, at least 15, at least 16, at least 17, at least 18, at least 19, or at least 20 mutations in the RBD. In embodiments, the RBD may contain any combination of modifications as shown in Table 1C.

TABLE 1C
Modifications to RBD (SEQ ID NO: 126)

| Position within | Position within | Position within | |
|---|---|---|---|
| SEQ ID NO: 1 | SEQ ID NO: 2 | SEQ ID NO: 126 | Potential Modifications |
| 339 | 326 | 9 | mutation to aspartic acid |
| | | | mutation to glutamic acid |
| | | | mutation to histidine |
| 346 | 333 | 16 | mutation to lysine |
| | | | mutation to arginine |
| | | | mutation to histidine |
| | | | mutation to threonine |
| | | | mutation to serine |
| 368 | 355 | 38 | mutation to isoleucine |
| | | | mutation to leucine |
| | | | mutation to valine |
| | | | mutation to beta-branched amino acid |
| 371 | 358 | 41 | mutation to leucine |
| | | | mutation to isoleucine |
| | | | mutation to valine |
| | | | mutation to phenylalanine |
| | | | mutation to tyrosine |
| | | | mutation to tryptophan |
| 373 | 360 | 43 | mutation to proline |
| 375 | 362 | 45 | mutation to phenylalanine |
| | | | mutation to tyrosine |
| | | | mutation to tryptophan |
| 376 | 363 | 46 | mutation to alanine |
| | | | mutation to glycine |
| 405 | 392 | 75 | mutation to asparagine |
| | | | mutation to glutamine |
| 408 | 395 | 78 | mutation to serine |
| | | | mutation to threonine |
| 417 | 404 | 87 | mutation to asparagine |
| | | | mutation to threonine |
| | | | mutation to isoleucine |
| | | | mutation to valine |
| | | | mutation to serine |
| | | | mutation to glutamine |
| | | | mutation to beta-branched amino acid |
| 432 | 419 | 102 | mutation to lysine |
| | | | mutation to arginine |
| | | | mutation to histidine |
| 439 | 426 | 109 | mutation to lysine |
| | | | mutation to arginine |
| | | | mutation to histidine |
| 440 | 427 | 110 | mutation to lysine |
| | | | mutation to arginine |
| | | | mutation to histidine |
| 444 | 431 | 114 | mutation to threonine |
| | | | mutation to serine |
| 445 | 432 | 115 | mutation to proline |
| 446 | 433 | 116 | mutation to serine |
| | | | mutation to threonine |
| 452 | 439 | 122 | mutation to arginine |
| | | | mutation to lysine |
| | | | mutation to histidine |
| | | | mutation to glutamine |
| | | | mutation to asparagine |
| 453 | 440 | 123 | mutation to phenylalanine |
| | | | mutation to tryptophan |
| 456 | 443 | 126 | mutation to leucine |
| | | | mutation to isoleucine |
| | | | mutation to valine |
| 460 | 447 | 130 | mutation to lysine |
| | | | mutation to arginine |
| 477 | 464 | 147 | mutation to asparagine |
| | | | mutation to glutamine |
| 478 | 465 | 148 | mutation to lysine |
| | | | mutation to arginine |
| | | | mutation to histidine |
| 484 | 471 | 154 | mutation to alanine |
| | | | mutation to lysine |
| | | | mutation to arginine |
| | | | mutation to histidine |
| | | | mutation to glutamine |
| | | | mutation to asparagine |
| 486 | 473 | 156 | mutation to valine |
| | | | mutation to leucine |
| | | | mutation to isoleucine |
| | | | mutation to serine |
| | | | mutation to threonine |
| 490 | 477 | 160 | mutation to serine |
| | | | mutation to threonine |
| 493 | 480 | 163 | mutation to lysine |
| | | | mutation to arginine |
| | | | mutation to histidine |
| 494 | 481 | 164 | mutation to proline |
| 496 | 483 | 166 | mutation to serine |
| | | | mutation to threonine |
| 498 | 485 | 168 | mutation to lysine |
| | | | mutation to arginine |
| | | | mutation to histidine |
| 501 | 488 | 171 | mutation to tyrosine |
| | | | mutation to phenylalanine |
| | | | mutation to tryptophan |
| 505 | 492 | 175 | mutation to histidine |
| 521 | 508 | 191 | mutation to serine |
| | | | mutation to threonine |

*amino acids 331-527 of SEQ ID NO: 1 and amino acids 318-514 of SEQ ID NO: 2

### CoV S Polypeptide Antigens—Modifications to SD1/2

In embodiments, the CoV S polypeptides contain one or more modifications to the SD1/2 having an amino acid sequence of SEQ ID NO: 122, which corresponds to amino acids 542-681 of SEQ ID NO: 1 or amino acids 529-668 of SEQ ID NO: 2.

The amino acid sequence of the SD1/2 (SEQ ID NO: 122) is shown below.

NFNGLTGTGVLTESNKKFLPFQQFGRDIADTTDAVRDPQTLEILDITPCS
FGGVSVITPGTNTSNQVAVLYQDVNCTEVPVAIHADQLTPTWRVYSTGSN
VFQTRAGCLIGAEHVNNSYECDIPIGAGICASYQTQTNSP

In embodiments, the CoV S polypeptides described herein comprise a SD1/2 with at least 95%, at least 96%, at least 97%, at least 98%, at least 99%, or at least 99.5%, identity to the SD1/2 of SEQ ID NO: 1 or SEQ ID NO: 2. The SD1/2 may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the SD1/2 of SEQ ID NO: 1 or SEQ ID NO: 2. The SD1/2 may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the SD1/2 of SEQ ID NO: 1 or SEQ ID NO: 2.

In embodiments, the CoV S polypeptide has at least one, at least two, at least three, at least four, at least four, at least 5, at least 6, at least 7, at least 8, at least 9, at least 10, at least 11, at least 12, at least 13, at least 14, at least 15, at least 16, at least 17, at least 18, at least 19, or at least 20 mutations in the SD1/2. In embodiments, the SD1/2 may contain any combination of modifications as shown in Table 1D.

TABLE 1D
Modifications to SD1/2 (SEQ ID NO: 122)

| Position within | Position within | Position within | |
|---|---|---|---|
| SEQ ID NO: 1 | SEQ ID NO: 2 | SEQ ID NO: 122 | Potential Modifications |
| 547 | 534 | 6 | mutation to lysine |
| | | | mutation to arginine |
| | | | mutation to histidine |
| 570 | 557 | 29 | mutation to aspartic acid |
| | | | mutation to glutamic acid |
| 604 | 591 | 63 | mutation to isoleucine |
| | | | mutation to leucine |
| | | | mutation to valine |
| | | | mutation to beta-branched amino acid |
| 613 | 600 | 72 | mutation to histidine |
| | | | mutation to lysine |
| | | | mutation to arginine |
| 614 | 601 | 73 | mutation to glycine |
| | | | mutation to alanine |
| 639 | 626 | 98 | mutation to valine |
| | | | mutation to leucine |
| | | | mutation to isoleucine |
| 655 | 642 | 114 | mutation to tyrosine |
| | | | mutation to phenylalanine |
| | | | mutation to tryptophan |
| 658 | 645 | 117 | mutation to serine |
| 677 | 664 | 136 | mutation to histidine |
| 679 | 666 | 138 | mutation to lysine |
| | | | mutation to arginine |
| | | | mutation to histidine |
| 681 | 668 | 140 | mutation to histidine |
| | | | mutation to lysine |
| | | | mutation to arginine |
| 547 | 534 | 6 | mutation to lysine |
| | | | mutation to arginine |
| | | | mutation to histidine |
| 570 | 557 | 29 | mutation to aspartic acid |
| | | | mutation to glutamic acid |
| 613 | 600 | 72 | mutation to histidine |
| | | | mutation to lysine |
| | | | mutation to arginine |
| 614 | 601 | 73 | mutation to glycine |
| | | | mutation to alanine |
| 655 | 642 | 114 | mutation to tyrosine |
| | | | mutation to phenylalanine |
| | | | mutation to tryptophan |
| 677 | 664 | 136 | mutation to histidine |
| 679 | 666 | 138 | mutation to lysine |
| | | | mutation to arginine |
| | | | mutation to histidine |

*amino acids 542-681 of SEQ ID NO: 1 or amino acids 529-668 of SEQ ID NO: 2

### CoV S Polypeptide Antigens-Modifications to Furin Cleavage Site

In embodiments, the CoV S polypeptides contain a furin site (RRAR), which corresponds to amino acids 682-685 of SEQ ID NO: 1 or amino acids 669-672 of SEQ ID NO: 2, that is inactivated by one or more mutations. Inactivation of the furin cleavage site prevents furin from cleaving the CoV S polypeptide. In embodiments, the CoV S polypeptides described herein which contain an inactivated furin cleavage site are expressed as a single chain.

In embodiments, one or more of the amino acids comprising the native furin cleavage site is mutated to any natural amino acid. In embodiments, the amino acids are L-amino acids. Non-limiting examples of amino acids include alanine, arginine, glycine, asparagine, aspartic acid, cysteine, glutamine, glutamic acid, serine, threonine, histidine, lysine, methionine, proline, valine, isoleucine, leucine, tyrosine, tryptophan, and phenylalanine.

In embodiments, one or more of the amino acids comprising the native furin cleavage site is mutated to glutamine. In embodiments, 1, 2, 3, or 4 amino acids may be mutated to glutamine. In embodiments, one of the arginines comprising the native furin cleavage site is mutated to glutamine. In embodiments, two of the arginines comprising the native furin cleavage site are mutated to glutamine. In embodiments, three of the arginines comprising the native furin cleavage site are mutated to glutamine.

In embodiments, one or more of the amino acids comprising the native furin cleavage site, is mutated to alanine. In embodiments, 1, 2, 3, or 4 amino acids may be mutated to alanine. embodiments, one of the arginines comprising the native furin cleavage site is mutated to alanine. In embodiments, two of the arginines comprising the native furin cleavage site are mutated to alanine. In embodiments, three of the arginines comprising the native furin cleavage site are mutated to alanine.

In embodiments, one or more of the amino acids comprising the native furin cleavage site is mutated to glycine. In embodiments, 1, 2, 3, or 4 amino acids may be mutated to glycine. In embodiments, one of the arginines of the native furin cleavage site is mutated to glycine. In embodiments, two of the arginines comprising the native furin cleavage site are mutated to glycine. In embodiments, three of the arginines comprising the native furin cleavage site are mutated to glycine.

In embodiments, one or more of the amino acids comprising the native furin cleavage site, is mutated to asparagine. For example 1, 2, 3, or 4 amino acids may be mutated to asparagine. In embodiments, one of the arginines comprising the native furin cleavage site is mutated to asparagine. In embodiments, two of the arginines comprising the native furin cleavage site are mutated to asparagine. In embodiments, three of the arginines comprising the native furin cleavage site are mutated to asparagine.

Non-limiting examples of the amino acid sequences of the inactivated furin sites contained within the CoV S polypeptides are found in Table 1E.

TABLE 1E

| | Active or |
|---|---|
| | Inactive |
| | Furin |
| Amino Acid Sequence | Cleavage |
| of Furin Cleavage Site | Site |
| RRAR (SEQ ID NO: 6) | Active |
| QQAQ (SEQ ID NO: 7) | Inactive |
| QRAR (SEQ ID NO: 8) | Inactive |
| RQAR (SEQ ID NO: 9) | Inactive |
| RRAQ (SEQ ID NO: 10) | Inactive |
| QQAR (SEQ ID NO: 11) | Inactive |
| RQAQ (SEQ ID NO: 12) | Inactive |
| QRAQ (SEQ ID NO: 13) | Inactive |
| NNAN (SEQ ID NO: 14) | Inactive |
| NRAR (SEQ ID NO: 15) | Inactive |
| RNAR (SEQ ID NO: 16) | Inactive |
| RRAN (SEQ ID NO: 17) | Inactive |
| NNAR (SEQ ID NO: 18) | Inactive |
| RNAN (SEQ ID NO: 19) | Inactive |
| NRAN (SEQ ID NO: 20) | Inactive |
| AAAA (SEQ ID NO: 21) | Inactive |
| ARAR (SEQ ID NO: 22) | Inactive |
| RAAR (SEQ ID NO: 23) | Inactive |
| RRAA (SEQ ID NO: 24) | Inactive |
| AAAR (SEQ ID NO: 25) | Inactive |
| RAAA (SEQ ID NO: 26) | Inactive |
| ARAA (SEQ ID NO: 27) | Inactive |
| GGAG (SEQ ID NO: 28) | Inactive |
| GRAR (SEQ ID NO: 29) | Inactive |
| RGAR (SEQ ID NO: 30) | Inactive |
| RRAG (SEQ ID NO: 31) | Inactive |
| GGAR (SEQ ID NO: 32) | Inactive |
| RGAG (SEQ ID NO: 33) | Inactive |
| GRAG (SEQ ID NO: 34) | Inactive |
| GSAS (SEQ ID NO: 97) | Inactive |
| GSGA (SEQ ID NO: 111) | Inactive |

In embodiments, in lieu of an active furin cleavage site (SEQ ID NO: 6) the CoV S polypeptides described herein contain an inactivated furin cleavage site. In embodiments, the amino acid sequence of the inactivated furin cleavage site is represented by any one of SEQ ID NO: 7-34 or SEQ ID NO: 97. In embodiments, the amino acid sequence of the inactivated furin cleavage site is QQAQ (SEQ ID NO: 7). In embodiments, the amino acid sequence of the inactivated furin cleavage site is GSAS (SEQ ID NO: 97). In embodiments, the amino acid sequence of the inactivated furin cleavage site is GSGA (SEQ ID NO: 111). In embodiments, the amino acid sequence of the inactivated furin cleavage site is GG, GGG (SEQ ID NO: 127), GGGG (SEQ ID NO: 128), or GGGGG (SEQ ID NO: 129).

### CoV S Polypeptide Antigens-Modifications to S2 Subunit

In embodiments, the CoV S polypeptides contain one or more modifications to the S2 subunit having an amino acid sequence of SEQ ID NO: 120, which corresponds to amino acids 686-1273 of SEQ ID NO: 1 or amino acids 673-1260 of SEQ ID NO: 2.

The amino acid sequence of the S2 subunit (SEQ ID NO: 120) is shown below.

SVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQV
KQIYKTPPIKDFGGFNFSQILPDPSKPSKRSFIEDLLFNKVTLADAGFIK
QYGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKIQ
DSLSSTASALGKLQDVVNQNAQALNTLVKQLSSNFGAISSVLNDILSRLD
KVEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNN
TVYDPLQPELDSFKEELDKYFKNHTSPDVDLGDISGINASVVNIQKEIDR
LNEVAKNLNESLIDLQELGKYEQYIKWPWYIWLGFIAGLIAIVMVTIMLC
CMTSCCSCLKGCCSCGSCCKFDEDDSEPVLKGVKLHYT

In embodiments, the CoV S polypeptides described herein comprise an S2 subunit with at least 95%, at least 96%, at least 97%, at least 98%, at least 99%, or at least 99.5%, identity to the S2 subunit of SEQ ID NO: 1 or SEQ ID NO: 2. The S2 subunit may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the S2 subunit of SEQ ID NO: 1 or SEQ ID NO: 2. The S2 subunit may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the S2 subunit of SEQ ID NO: 1 or SEQ ID NO: 2.

In embodiments, the S2 subunit may contain any combination of modifications as shown in Table 1F.

TABLE 1F
Modifications to S2 (SEQ ID NO: 120)

| Position within | Position within | Position within | |
|---|---|---|---|
| SEQ ID NO: 1 | SEQ ID NO: 2 | SEQ ID NO: 120 | Possible Modifications |
| 689-698 | 676-685 | 4-13 | Deletion of up to about 1, up to about 2, up to |
| | | | about 3, up to about 4, up to about 5, up to about |
| | | | 6, up to about 7, up to about 8, up to about 9, or up |
| | | | to about 10 amino acids |
| 701 | 688 | 16 | Mutation to beta-branched amino acid |
| | | | Mutation to valine |
| | | | Mutation to isoleucine |
| | | | Mutation to threonine |
| 704 | 691 | 19 | Mutation to leucine |
| | | | Mutation to isoleucine |
| | | | Mutation to valine |
| 715-724 | 702-711 | 30-39 | Deletion of up to about 1, up to about 2, up to |
| | | | about 3, up to about 4, up to about 5, up to about |
| | | | 6, up to about 7, up to about 8, up to about 9, or up |
| | | | to about 10 amino acids |
| 716 | 703 | 31 | Mutation to beta-branched amino acid |
| | | | Mutation to valine |
| | | | Mutation to isoleucine |
| 764 | 751 | 79 | Mutation to lysine |
| | | | Mutation to arginine |
| | | | Mutation to histidine |
| 788-806 | 775-793 | 103-121 | Deletion of up to about 1, up to about 2, up to |
| | | | about 3, up to about 4, up to about 5, up to about |
| | | | 6, up to about 7, up to about 8, up to about 9, up to |
| | | | about 10, up to about 11, up to about 12, up to |
| | | | about 13, up to about 14, up to about 15, up to |
| | | | about 16, up to about 17, up to about 18, or up to |
| | | | about 19 amino acids |
| 796 | 783 | 111 | Mutation to tyrosine |
| | | | Mutation to phenylalanine |
| | | | Mutation to tryptophan |
| 819-828 | 806-815 | 134-143 | Deletion of up to about 1, up to about 2, up to |
| | | | about 3, up to about 4, up to about 5, up to about |
| | | | 6, up to about 7, up to about 8, up to about 9, or up |
| | | | to about 10 amino acids |
| 856 | 843 | 171 | Mutation to lysine |
| | | | Mutation to arginine |
| | | | Mutation to histidine |
| 859 | 846 | 174 | Mutation to asparagine |
| | | | Mutation to glutamine |
| 888 | 875 | 203 | Mutation to leucine |
| | | | Mutation to isoleucine |
| | | | Mutation to valine |
| 950 | 937 | 265 | Mutation to asparagine |
| | | | Mutation to glutamine |
| 954 | 941 | 269 | Mutation to histidine |
| | | | Mutation to lysine |
| | | | Mutation to arginine |
| 969 | 956 | 284 | Mutation to lysine |
| | | | Mutation to arginine |
| | | | Mutation to histidine |
| 981 | 968 | 296 | Mutation to phenylalanine |
| | | | Mutation to tyrosine |
| | | | Mutation to tryptophan |
| 982 | 969 | 297 | Mutation to alanine |
| | | | Mutation to glycine |
| | | | Mutation to threonine |
| 986 | 973 | 301 | Mutation to proline |
| | | | Mutation to glycine |
| 987 | 974 | 302 | Mutation to proline |
| | | | Mutation to glycine |
| 1027 | 1014 | 342 | Mutation to isoleucine |
| | | | Mutation to valine |
| | | | Mutation to serine |
| 1071 | 1058 | 386 | Mutation to histidine |
| | | | Mutation to arginine |
| | | | Mutation to lysine |
| 1118 | 1105 | 433 | Mutation to histidine |
| | | | Mutation to lysine |
| | | | Mutation to arginine |
| | | | Mutation to asparagine |
| | | | Mutation to glutamine |
| 1176 | 1163 | 491 | Mutation to phenylalanine |
| | | | Mutation to tyrosine |
| | | | Mutation to tryptophan |
| 1199 | 1186 | 514 | Mutation to asparagine |
| | | | Mutation to glutamine |
| 1214-1237 | 1201-1224 | 1-24 | Deletion of one or more amino acids of TM |
| 1238-1273 | 1225-1260 | 1-36 | Deletion of one or more amino acids of CD |

*amino acids 686-1273 of SEQ ID NO: 1 and amino acids 673-1260 of SEQ ID NO: 2

In embodiments, the CoV S polypeptides contain a deletion, corresponding to one or more deletions within amino acids 676-685 of the native CoV Spike(S) polypeptide (SEQ ID NO: 2). In embodiments, 1, 2, 3, 4, 5, 6, 7, 8, 9, or 10 amino acids of amino acids 676-685 of the native CoV Spike(S) polypeptide (SEQ ID NO:2) are deleted. In embodiments, the deletions of amino acids within amino acids 676-685 are consecutive e.g. amino acids 676 and 677 are deleted or amino acids 680 and 681 are deleted. In embodiments, the deletions of amino acids within amino acids 676-685 are non-consecutive e.g. amino acids 676 and 680 are deleted or amino acids 677 and 682 are deleted. In embodiments, CoV S polypeptides containing a deletion, corresponding to one or more deletions within amino acids 676-685, have an amino acid sequence selected from the group consisting of SEQ ID NO: 62 and SEQ ID NO: 63.

In embodiments, the CoV S polypeptides contain a deletion, corresponding to one or more deletions within amino acids 702-711 of the native CoV Spike(S) polypeptide (SEQ ID NO: 2). In embodiments, 1, 2, 3, 4, 5, 6, 7, 8, 9, or 10 amino acids of amino acids 702-711 of the native SARS-COV-2 Spike(S) polypeptide (SEQ ID NO:2) are deleted. In embodiments, the one or more deletions of amino acids within amino acids 702-711 are consecutive e.g. amino acids 702 and 703 are deleted or amino acids 708 and 709 are deleted. In embodiments, the deletions of amino acids within amino acids 702-711 are non-consecutive e.g. amino acids 702 and 704 are deleted or amino acids 707 and 710 are deleted. In embodiments, the CoV S polypeptides containing a deletion, corresponding to one or more deletions within amino acids 702-711, have an amino acid sequence selected from the group consisting of SEQ ID NO: 64 and SEQ ID NO: 65.

In embodiments, the CoV S polypeptides contain a deletion, corresponding to one or more deletions within amino acids 775-793 of the native CoV S polypeptide (SEQ ID NO: 2). In embodiments, up to about 1, 2, 3, 4, 5, 6, 7, 8, 9, 10, 11, 12, 13, 14, 15, 16, 17, 18, or 19 amino acids of amino acids 775-793 of the native SARS-COV-2 Spike(S) polypeptide (SEQ ID NO:2) are deleted. In embodiments, the one or more deletions of amino acids within amino acids 775-793 are consecutive e.g. amino acids 776 and 777 are deleted or amino acids 780 and 781 are deleted. In embodiments, the deletions of amino acids within amino acids 775-793 are non-consecutive e.g. amino acids 775 and 790 are deleted or amino acids 777 and 781 are deleted.

In embodiments, the CoV S polypeptides contain a deletion of the fusion peptide (SEQ ID NO: 104), which corresponds to amino acids 806-815 of SEQ ID NO: 2. In embodiments, 1, 2, 3, 4, 5, 6, 7, 8, 9, or 10 amino acids of the fusion peptide of the CoV Spike(S) polypeptide (SEQ ID NO: 2) are deleted. In embodiments, the deletions of amino acids within the fusion peptide arc consecutive e.g. amino acids 806 and 807 are deleted or amino acids 809 and 810 are deleted. In embodiments, the deletions of amino acids within the fusion peptide are non-consecutive e.g. amino acids 806 and 808 are deleted or amino acids 810 and 813 are deleted. In embodiments, the CoV S polypeptides containing a deletion, corresponding to one or more amino acids of the fusion peptide, have an amino acid sequence selected from SEQ ID NOS: 66, 77, and 105-108.

In embodiments, the CoV S polypeptides contain a mutation at Lys-973 of the native CoV Spike(S) polypeptide (SEQ ID NO: 2). In embodiments, Lys-973 is mutated to any natural amino acid. In embodiments, Lys-973 is mutated to proline. In embodiments, Lys-973 is mutated to glycine. In embodiments, the CoV S polypeptides containing a mutation at amino acid 973 are selected from the group consisting of SEQ ID NO: 84-89, 105-106, and 109-110.

In embodiments, the CoV S polypeptides contain a mutation at Val-974 of the native CoV Spike(S) polypeptide (SEQ ID NO: 2). In embodiments, Val-974 is mutated to any natural amino acid. In embodiments, Val-974 is mutated to proline. In embodiments, Val-974 is mutated to glycine. In embodiments, the CoV S polypeptides containing a mutation at amino acid 974 arc selected from the group consisting of SEQ ID NO: 84-89, 105-106, and 109-110.

In embodiments, the CoV S polypeptides contain a mutation at Lys-973 and Val-974 of the native CoV Spike(S) polypeptide (SEQ ID NO: 2). In embodiments, Lys-973 and Val-974 arc mutated to any natural amino acid. In embodiments, Lys-973 and Val-974 are mutated to proline. In embodiments, the CoV S polypeptides containing a mutation at amino acids 973 and 974 are selected from SEQ ID NOS: 84-89, 105-106, 109-110, 175, 220, and 217-228.

CoV S Polypeptide Antigens-Modifications to S2 subunit-HR1 Domain

In embodiments, the CoV S polypeptides contain one or more modifications to the HR1 domain having an amino acid sequence of SEQ ID NO: 119, which corresponds to amino acids 912-984 of SEQ ID NO: 1 or amino acids 889-971 of SEQ ID NO: 2.

The amino acid sequence of the HR1 domain (SEQ ID NO: 119) is shown below.

MAYRFNGIGVTQNVLYENQKLIANQFNSAIGKIQDSLSSTASALGKLQDV
VNQNAQALNTLVKQLSSNFGAISSVLNDILSRL

In embodiments, the CoV S polypeptides described herein comprise an HR1 domain with at least 95%, at least 96%, at least 97%, at least 98%, at least 99%, or at least 99.5%, identity to the HR1 domain of SEQ ID NO: 1 or SEQ ID NO: 2. The HR1 domain may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the HR1 domain of SEQ ID NO: 1 or SEQ ID NO: 2. The HR1 domain may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the HR1 domain of SEQ ID NO: 1 or SEQ ID NO: 2.

In embodiments, the HR1 domain may contain any combination of modifications as shown in Table 1G.

TABLE 1G
Modifications to HR1 (SEQ ID NO: 119)

| Position | Position | Position | |
|---|---|---|---|
| within | within | within | |
| SEQ ID | SEQ ID | SEQ ID | |
| NO: 1 | NO: 2 | NO: 119 | Possible Modifications |
| 950 | 937 | 49 | Mutation to asparagine |
| | | | Mutation to glutamine |
| 954 | 941 | 53 | Mutation to histidine |
| 969 | 956 | 68 | Mutation to lysine |
| | | | Mutation to arginine |
| | | | Mutation to histidine |
| 981 | 968 | 80 | Mutation to phenylalanine |
| | | | Mutation to tyrosine |
| | | | Mutation to tryptophan |
| 982 | 969 | 81 | Mutation to alanine |
| | | | Mutation to glycine |
| | | | Mutation to threonine |

* amino acids 912-984 of SEQ ID NO: 1 and amino acids 889-971 of SEQ ID NO: 2)

### CoV S Polypeptide Antigens-Modifications to S2 Subunit-HR2 Domain

In embodiments, the CoV S polypeptides contain one or more modifications to the HR2 domain having an amino acid sequence of SEQ ID NO: 125, which corresponds to amino acids 1163-1213 of SEQ ID NO: 1 or amino acids 1150-1200 of SEQ ID NO: 2.

The amino acid sequence of the HR2 domain (SEQ ID NO: 125) is shown below.
DVDLGDISGINASVVNIQKEIDRLNEVAKNLNESLIDLQELGKYEQYIKWP

In embodiments, the CoV S polypeptides described herein comprise an HR2 domain with at least 95%, at least 96%, at least 97%, at least 98%, at least 99%, or at least 99.5%, identity to the HR2 domain of SEQ ID NO: 1 or SEQ ID NO: 2. The HR2 domain may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the HR2 domain of SEQ ID NO: 1 or SEQ ID NO: 2. The HR2 domain may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the HR2 domain of SEQ ID NO: 1 or SEQ ID NO: 2.

CoV S Polypeptide Antigens-Modifications to the TM domain

In embodiments, the CoV S polypeptides contain one or more modifications to the TM domain having an amino acid sequence of SEQ ID NO: 123, which corresponds to amino acids 1214-1237 of SEQ ID NO: 1 or amino acids 1201-1224 of SEQ ID NO: 2.

The amino acid sequence of the TM domain (SEQ ID NO: 123) is shown below.

WYIWLGFIAGLIAIVMVTIMLCCM

In embodiments, the CoV S polypeptides described herein comprise a TM domain with at least 95%, at least 96%, at least 97%, at least 98%, at least 99%, or at least 99.5%, identity to the TM domain of SEQ ID NO: 1 or SEQ ID NO: 2. The TM domain may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the TM domain of SEQ ID NO: 1 or SEQ ID NO: 2. The TM domain may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the TM domain of SEQ ID NO: 1 or SEQ ID NO: 2.

In embodiments, the CoV S polypeptides described herein lack the entire TM domain. In embodiments, the CoV S polypeptides comprise the TM domain.

### CoV S Polypeptide Antigens-Modifications to the CT

In embodiments, the CoV S polypeptides contain one or more modifications to the CT having an amino acid sequence of SEQ ID NO: 124, which corresponds to amino acids 1238-1273 of SEQ ID NO: 1 or amino acids 1225-1260 of SEQ ID NO: 2.

The amino acid sequence of the CT (SEQ ID NO: 124) is shown below:

TSCCSCLKGCCSCGSCCKFDEDDSEPVLKGVKLHYT

In embodiments, the CoV S polypeptides described herein comprise a CT with at least 95%, at least 96%, at least 97%, at least 98%, at least 99%, or at least 99.5%, identity to the CT of SEQ ID NO: 1 or SEQ ID NO: 2. The CT may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the CT of SEQ ID NO: 1 or SEQ ID NO: 2. The CT may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the CT of SEQ ID NO: 1 or SEQ ID NO: 2.

In embodiments, the CoV S polypeptides described herein lack a CT. In embodiments, the CoV S polypeptides comprise the CT.

In embodiments, the CoV S polypeptides comprise a TM and a CT. In embodiments, the CoV Spike(S) polypeptides contain a deletion of one or more amino acids from the transmembrane and cytoplasmic tail (TMCT) (corresponding to amino acids 1201-1260). The amino acid sequence of the TMCT is represented by SEQ ID NO: 39. In embodiments, the CoV S polypeptides which have a deletion of one or more residues of the TMCT have enhanced protein expression. In embodiments, the CoV Spike(S) polypeptides which have one or more deletions from the TMCT have an amino acid sequence selected from the group consisting of SEQ ID NO: 40, 41, 42, 52, 54, 59, 61, 88, and 89. In embodiments, the CoV S polypeptides which have one or more deletions from the TM-CD are encoded by an isolated nucleic acid sequence selected from the group consisting of SEQ ID NO: 39, 43, 53, and 60.

### CoV S Polypeptide Antigens-Non-Limiting Combinations of Mutations

In embodiments, the CoV S polypeptides contain a deletion of amino acids 56 and 57 of the native CoV Spike(S) polypeptide (SEQ ID NO: 2).

In embodiments, the CoV S polypeptides contain deletions of amino acids 131 and 132 of the native CoV Spike(S) polypeptide (SEQ ID NO: 2).

In embodiments, the CoV S polypeptides contain a deletion of amino acids 56 and 131 of the native CoV Spike(S) polypeptide (SEQ ID NO: 2). In embodiments, the CoV S polypeptides contain a deletion of amino acids 57 and 131 of the native CoV Spike(S) polypeptide (SEQ ID NO: 2).

In embodiments, the CoV S polypeptides contain a deletion of amino acids 56, 57, and 131 of the native CoV Spike(S) polypeptide (SEQ ID NO: 2).

In embodiments, the CoV S polypeptides contain a deletion of amino acids 56 and 132 of the native CoV Spike(S) polypeptide (SEQ ID NO: 2).

In embodiments, the CoV S polypeptides contain a deletion of amino acids 57 and 132 of the native CoV Spike(S) polypeptide (SEQ ID NO: 2).

In embodiments, the CoV S polypeptides contain a deletion of amino acids 56, 57, and 132 of the native CoV Spike(S) polypeptide (SEQ ID NO: 2).

In embodiments, the CoV S polypeptides contain a deletion of amino acids 56, 57, 131, and 132 of the native CoV Spike(S) polypeptide (SEQ ID NO: 2).

In embodiments, the CoV S polypeptides contain mutations that stabilize the prefusion conformation of the CoV S polypeptide. In embodiments, the CoV S polypeptides contain proline or glycine substitutions which stabilize the prefusion conformation. This strategy has been utilized for to develop a prefusion stabilized MERS-COV S protein as described in the following documents which are each incorporated by reference herein in their entirety: Proc Natl Acad Sci USA. 2017 Aug. 29; 114 (35): E7348-E7357; Sci Rep. 2018 Oct. 24; 8 (1): 15701; U.S. Publication No. 2020/0061185; and PCT Application No. PCT/US2017/058370.

In embodiments, the CoV S polypeptides contain a mutation at Lys-973 and Val-974 and an inactivated furin cleavage site. In embodiments, the CoV S polypeptides contain mutations of Lys-973 and Val-974 to proline and an inactivated furin cleavage site, having the amino acid sequence of QQAQ (SEQ ID NO: 7) or GSAS (SEQ ID NO: 96). In embodiments, the CoV S polypeptides containing mutations of Lys-973 and Val-974 to proline and an inactivated furin cleavage site have an amino acid sequences of SEQ ID NOS: 86 or 87 and a nucleic acid sequence of SEQ ID NO: 96.

In embodiments, the CoV S polypeptides contain a mutation at Lys-973 and Val-974, an inactivated furin cleavage site, and a deletion of one or more amino acids of the fusion peptide. In embodiments, the CoV S polypeptides contain mutations of Lys-973 and Val-974 to proline, an inactivated furin cleavage site having the amino acid sequence of QQAQ (SEQ ID NO: 7) or GSAS (SEQ ID NO: 96), and deletion of one or more amino acids of the fusion peptide. In embodiments, the CoV S polypeptides containing mutations of Lys-973 and Val-974 to proline, an inactivated furin cleavage site, and deletion of one or more amino acids of the fusion peptide having an amino acid sequence of SEQ ID NO: 105 or 106. In embodiments, the CoV S polypeptide contains a mutation of Leu-5 to phenylalanine, mutation of Thr-7 to asparagine, mutation of Pro-13 to serine, mutation of Asp-125 to tyrosine, mutation of Arg-177 to serine, mutation of Lys-404 to threonine, mutation of Glu-471 to lysine, mutation of Asn-488 to tyrosine, mutation of His-642 to tyrosine, mutation of Thr-1014 to isoleucine, mutations of Lys-973 and Val-974 to proline, and an inactivated furin cleavage site having the amino acid sequence of QQAQ (SEQ ID NO: 7) or GSAS (SEQ ID NO: 96) relative to the native CoV Spike(S) polypeptide (SEQ ID NO: 2).

In embodiments, the CoV S polypeptide contains a mutation of Trp-139 to cysteine, mutation of Leu-439 to arginine, mutations of Lys-973 and Val-974 to proline, and an inactivated furin cleavage site having the amino acid sequence of QQAQ (SEQ ID NO: 7) or GSAS (SEQ ID NO: 96) relative to the native CoV Spike(S) polypeptide (SEQ ID NO: 2). In embodiments, the CoV S polypeptide contains a mutation of Trp-152 to cysteine, mutation of Leu-452 to arginine, mutation of Ser-13 to isoleucine, mutations of Lys-986 and Val-987 to proline, and an inactivated furin cleavage site having the amino acid sequence of QQAQ (SEQ ID NO: 7) or GSAS (SEQ ID NO: 96) relative to the native CoV Spike(S) polypeptide (SEQ ID NO: 1).

In embodiments, the CoV S polypeptide contains a mutation of Lys-404 to threonine or asparagine, mutation of Glu-471 to lysine, mutation of Asn-488 to tyrosine, mutation of Leu-5 to phenylalanine, mutation of Asp-67 to alanine, mutation of Asp-202 to glycine, deletion of one or more of amino acids 229-231, mutation of Arg-233 to isoleucine, mutations of Lys-973 and Val-974 to proline, and an inactivated furin cleavage site having the amino acid sequence of QQAQ (SEQ ID NO: 7) or GSAS (SEQ ID NO: 96) relative to the native CoV Spike(S) polypeptide (SEQ ID NO: 2).

In embodiments, the CoV S polypeptide contains a mutation of Asn-488 to tyrosine, mutations of Lys-973 and Val-974 to proline, and an inactivated furin cleavage site having the amino acid sequence of QQAQ (SEQ ID NO: 7) or GSAS (SEQ ID NO: 96) relative to the native CoV Spike(S) polypeptide (SEQ ID NO: 2). In embodiments, the CoV S polypeptide having a mutation of Asn-488 to tyrosine, mutations of Lys-973 and Val-974 to proline, and an inactivated furin cleavage site having the amino acid sequence of QQAQ (SEQ ID NO: 7) or GSAS (SEQ ID NO: 96) comprises an amino acid sequence of SEQ ID NO: 112.

In embodiments, the CoV S polypeptide contains a mutation of Asp-601 to glycine, a mutation of Asn-488 to tyrosine, mutations of Lys-973 and Val-974 to proline, and an inactivated furin cleavage site having the amino acid sequence of QQAQ (SEQ ID NO: 7) or GSAS (SEQ ID NO: 96) relative to the native CoV Spike(S) polypeptide (SEQ ID NO: 2). In embodiments, the CoV S polypeptide having a mutation of Asn-488 to tyrosine, mutations of Lys-973 and Val-974 to proline, and an inactivated furin cleavage site having the amino acid sequence of QQAQ (SEQ ID NO: 7) or GSAS (SEQ ID NO: 96) comprises an amino acid sequence of SEQ ID NO: 113.

In embodiments, the CoV S polypeptide contains deletion of amino acids 56, 57, and 131, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of QQAQ (SEQ ID NO: 7), GSAS (SEQ ID NO: 96), or GG relative to the native CoV Spike(S) polypeptide (SEQ ID NO: 2). In embodiments, the CoV S polypeptide having deletion of amino acids 56, 57, and 131, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of QQAQ (SEQ ID NO: 7) or GSAS (SEQ ID NO: 96) comprises an amino acid sequence of SEQ ID NO: 114. In embodiments, the CoV S polypeptide having deletion of amino acids 56, 57, and 131, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of QQAQ (SEQ ID NO: 7) or GSAS (SEQ ID NO: 96) or GG comprises an amino acid sequence of SEQ ID NO: 136. In embodiments, the CoV S polypeptide having deletion of amino acids 56, 57, and 131, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of GG comprises an amino acid sequence of SEQ ID NO: 137 or SEQ ID NO: 138. In some embodiments, the CoV S polypeptide having an amino acid sequence of SEQ ID NO: 114 or SEQ ID NO: 136 is encoded by a nucleic acid having a nucleic acid sequence of SEQ ID NO: 135. In some embodiments, the CoV S polypeptide having an amino acid sequence of SEQ ID NO: 137 or SEQ ID NO: 138 is encoded by a nucleic acid having a sequence of SEQ ID NO: 139.

In embodiments, the CoV S polypeptide contains deletion of amino acids 56, 57, and 132, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of QQAQ (SEQ ID NO: 7) or GSAS (SEQ ID NO: 96 relative to the native CoV Spike(S) polypeptide (SEQ ID NO: 2). In embodiments, the CoV S polypeptide having a deletion of amino acids 56, 57, and 132, mutation of Asn-488 to tyrosine, a mutation of Ala-557 to aspartate, mutation of Asp-601 to glycine, mutation of Pro-668 to histidine, mutation of Thr-703 to isoleucine, mutation of Ser-969 to alanine, mutation of Asp-1105 to histidine, mutations of Lys-973 and Val-974 to proline, and an inactivated furin cleavage site having the amino acid sequence of QQAQ (SEQ ID NO: 7) or GSAS (SEQ ID NO: 96) comprises an amino acid sequence of SEQ ID NO: 114.

In embodiments, the CoV S polypeptide contains mutation of Asn-488 to tyrosine, mutation of Asp-67 to alanine, mutation of Leu-229 to histidine, mutation of Asp-202 to glycine, mutation of Lys-404 to asparagine, mutation of Glu-471 to lysine, mutation of Ala-688 to valine, mutation of Asp-601 to glycine, mutations of Lys-973 and Val-974 to proline, and an inactivated furin cleavage site having the amino acid sequence of QQAQ (SEQ ID NO: 7) or GSAS (SEQ ID NO: 96) relative to the native CoV Spike(S) polypeptide (SEQ ID NO: 2). In embodiments, the CoV S polypeptide having a mutation of Asn-488 to tyrosine, mutation of Asp-67 to alanine, mutation of Leu-229 to histidine, mutation of Asp-202 to glycine, mutation of Lys-404 to asparagine, mutation of Glu-471 to lysine, mutation of Ala-688 to valine, mutation of Asp-601 to glycine, mutations of Lys-973 and Val-974 to proline, and an inactivated furin cleavage site having the amino acid sequence of QQAQ (SEQ ID NO: 7) or GSAS (SEQ ID NO: 96) comprises an amino acid sequence of SEQ ID NO: 115.

In embodiments, the CoV S polypeptide contains one or more modifications selected from: K973P, V974P, an inactivated furin cleavage site, deletions of amino acid 56, deletion of amino acid 57, deletion of amino acid 131, N488Y, A557D, D601G, P668H, T7031, S969A, and D1105H, wherein the amino acids are numbered with respect to a CoV S polypeptide having an amino acid sequence of SEQ ID NO: 2. In embodiments, the inactivated furin cleavage site has the amino acid sequence of QQAQ (SEQ ID NO: 7). In embodiments, the inactivated furin cleavage site has the amino acid sequence of GG.

In embodiments, the CoV S polypeptide contains one or more modifications selected from: K973P, V974P, an inactivated furin cleavage site, D67A, D202G, L229H, K404N, E471K, N488Y, D601G, and A688V, wherein the amino acids are numbered with respect to a CoV S polypeptide having an amino acid sequence of SEQ ID NO: 2. In embodiments, the inactivated furin cleavage site has the amino acid sequence of QQAQ (SEQ ID NO: 7). In embodiments, the inactivated furin cleavage site has the amino acid sequence of GG.

In embodiments, the CoV S polypeptide contains one or more modifications selected from: K973P, V974P, an inactivated furin cleavage site, deletion of amino acids 229-231, D67A, D202G, K404N, E471K, N488Y, D601G, and A688V, wherein the amino acids are numbered with respect to a CoV S polypeptide having an amino acid sequence of SEQ ID NO: 2.

In embodiments, the CoV S polypeptide contains one or more modifications selected from: K973P, V974P, an inactivated furin cleavage site having the amino acid sequence of QQAQ (SEQ ID NO: 7), deletion of amino acids 229-231, L5F, D67A, D202G, K404N, E471K, N488Y, D601G, and A688V, wherein the amino acids are numbered with respect to a CoV S polypeptide having an amino acid sequence of SEQ ID NO: 2. In embodiments, the CoV S polypeptide having one or more modifications selected from K973P, V974P, an inactivated furin cleavage site having the amino acid sequence of QQAQ (SEQ ID NO: 7), deletion of amino acids 229-231, L5F, D67A, D202G, K404N, E471K, N488Y, D601G, and A688V, wherein the amino acids are numbered with respect to a CoV S polypeptide having an amino acid sequence of SEQ ID NO: 2 comprises the amino acid sequence of SEQ ID NO: 144. In embodiments, the CoV S polypeptide having the amino acid sequence of SEQ ID NO: 144 is encoded by a nucleic acid having a sequence of SEQ ID NO: 145.

In embodiments, the CoV S polypeptide contains one or more modifications selected from: K973P, V974P, an inactivated furin cleavage site having the amino acid sequence of GG, deletion of amino acids 229-231, L5F, D67A, D202G, K404N, E471K, N488Y, D601G, and A688V, wherein the amino acids are numbered with respect to a CoV S polypeptide having an amino acid sequence of SEQ ID NO: 2. In embodiments, the CoV S polypeptide having one or more modifications selected from K973P, V974P, an inactivated furin cleavage site having the amino acid sequence of GG, deletion of amino acids 229-231, L5F, D67A, D202G, K404N, E471K, N488Y, D601G, and A688V, wherein the amino acids are numbered with respect to a CoV S polypeptide having an amino acid sequence of SEQ ID NO: 2 comprises the amino acid sequence of SEQ ID NO: 144. In embodiments, the CoV S polypeptide having the amino acid sequence of SEQ ID NO: 144 is encoded by a nucleic acid having a sequence of SEQ ID NO: 145.

In embodiments, the CoV S polypeptide contains one or more modifications selected from: K973P, V974P, an inactivated furin cleavage site, L5F, T7N, P13S, D125Y, R177S, K404T, E471K, N488Y, D601G, H642Y, T1014I, and V1163F, wherein the amino acids are numbered with respect to a CoV S polypeptide having an amino acid sequence of SEQ ID NO: 2. In embodiments, the CoV S polypeptide containing one or more modifications selected from: K973P, V974P, an inactivated furin cleavage site, L5F, T7N, P13S, D125Y, R177S, K404T, E471K, N488Y, D601G, H642Y, T1014I, and V1163F, wherein the amino acids are numbered with respect to a CoV S polypeptide having an amino acid sequence of SEQ ID NO: 2, has an amino acid sequence of SEQ ID NO: 151. In embodiments, the CoV S polypeptide having an amino acid sequence of SEQ ID NO: 151 is encoded by a nucleic acid having a sequence of SEQ ID NO: 150.

In embodiments, the CoV S polypeptide contains one or more modifications selected from: K973P, V974P, an inactivated furin cleavage site, deletion of amino acids 229-231, L5F, D67A, D202G, L229H, K404N, E471K, N488Y, D601G, and A688V, wherein the amino acids are numbered with respect to a CoV S polypeptide having an amino acid sequence of SEQ ID NO: 2.

In embodiments, the CoV S polypeptide contains one or more modifications selected from: K973P, V974P, an inactivated furin cleavage site, K404N, E471K, N488Y, L5F, D67A, D202G, L229H, D601G, A688V, and deletion of amino acids 229-231, wherein the amino acids are numbered with respect to a CoV S polypeptide having an amino acid sequence of SEQ ID NO: 2. In embodiments, the inactivated furin cleavage site has the amino acid sequence of QQAQ (SEQ ID NO: 7). In embodiments, the inactivated furin cleavage site has the amino acid sequence of GG.

In embodiments, the CoV S polypeptide contains one or more modifications selected from: K973P, V974P, an inactivated furin cleavage site, K404N, E471K, and N488K wherein the amino acids are numbered with respect to a CoV S polypeptide having an amino acid sequence of SEQ ID NO: 2. In embodiments, the CoV S polypeptide contains one or more modifications selected from: K973P, V974P, an inactivated furin cleavage site, K404N, E471K, and N488Y. In embodiments, the CoV S polypeptide is the RBD of the CoV S polypeptide having one or more modifications selected from K973P, V974P, an inactivated furin cleavage site, K404N, E471K, and N488K wherein the amino acids are numbered with respect to a CoV S polypeptide having an amino acid sequence of SEQ ID NO: 2. In embodiments, the CoV S polypeptide is the RBD of the CoV S polypeptide having one or more modifications selected from K973P, V974P, an inactivated furin cleavage site, K404N, E471K, and N488Y wherein the amino acids are numbered with respect to a CoV S polypeptide having an amino acid sequence of SEQ ID NO: 2.

In embodiments, the CoV S polypeptide contains one or more modifications selected from: K973P, V974P, an inactivated furin cleavage site having the amino acid sequence of GG, D601G, E404N, E471K, and N488Y. In embodiments, the CoV S polypeptide contains one or more modifications selected from: K973P, V974P, an inactivated furin cleavage site having the amino acid sequence of GG, and a D601G mutation, wherein the amino acids are numbered with respect to a CoV S polypeptide having an amino acid sequence of SEQ ID NO: 2. In embodiments, the CoV S polypeptide containing modifications selected from: K973P, V974P, an inactivated furin cleavage site having the amino acid sequence of GG, and a D601G mutation has an amino acid sequence of SEQ ID NO: 133.

In embodiments, the CoV S polypeptide contains one or more modifications selected from: K973P, V974P, an inactivated furin cleavage site, optionally wherein the inactivated furin cleavage site is QQAQ (SEQ ID NO: 7) or GG, K404N, E471K, N488K, D67A, D202G, L229H, D601G, and A688V, wherein the amino acids are numbered with respect to a CoV S polypeptide having an amino acid sequence of SEQ ID NO: 2. In embodiments, the CoV S polypeptide containing one or more modifications selected from: K973P, V974P, an inactivated furin cleavage site, optionally wherein the inactivated furin cleavage site is QQAQ (SEQ ID NO: 7) or GG, K404N, E471K, N488K, D67A, D202G, L229H, D601G, and A688V has an amino acid sequence of SEQ ID NO: 132 or SEQ ID NO: 141. In embodiments, the CoV S polypeptide having an amino acid sequence of SEQ ID NO: 132 is encoded by a nucleic acid having a nucleic acid sequence of SEQ ID NO: 131. In embodiments, the CoV S polypeptide having an amino acid sequence of SEQ ID NO: 132 is encoded by a nucleic acid having a nucleic acid sequence of SEQ ID NO: 142.

In embodiments, the CoV S polypeptide contains one or more modifications selected from: K973P, V974P, an inactivated furin cleavage site, W139C and L439R, wherein the amino acids are numbered with respect to a CoV S polypeptide having an amino acid sequence of SEQ ID NO: 2. In embodiments, the CoV S polypeptide comprising K973P, V974P, an inactivated furin cleavage site, W139C and L439R modifications is expressed with a signal peptide having an amino acid sequence of SEQ ID NO: 117 or SEQ ID NO: 5. In embodiments, the CoV S polypeptide comprises one or more modifications selected from: K973P, V974P, an inactivated furin cleavage site, D601G, W139C, and L439R, wherein the amino acids are numbered with respect to a CoV S polypeptide having an amino acid sequence of SEQ ID NO: 2. In embodiments, the CoV S polypeptide comprises K973P, V974P, an inactivated furin cleavage site, D601G, W139C, and L439R modifications and is expressed with a signal peptide having an amino acid sequence of SEQ ID NO: 117 or SEQ ID NO: 5.

In embodiments, the CoV S polypeptide comprises one or more modifications selected from: K973P, V974P, an inactivated furin cleavage site, D601G, L5F, D67A, D202G, deletions of amino acids 229-231, R233I, K404N, E471K, N488Y, and A688V, wherein the amino acids are numbered with respect to a CoV S polypeptide having an amino acid sequence of SEQ ID NO: 2.

In embodiments, the CoV S polypeptide contains one or more modifications selected from: K973P, V974P, an inactivated furin cleavage site, optionally wherein the inactivated furin cleavage site is QQAQ (SEQ ID NO: 7), W139C, S481P, D601G, and L439R, wherein the amino acids are numbered with respect to a CoV S polypeptide having an amino acid sequence of SEQ ID NO: 2. In embodiments, the CoV S polypeptide contains one or more modifications selected from: K973P, V974P, an inactivated furin cleavage site, optionally wherein the inactivated furin cleavage site is QQAQ (SEQ ID NO: 7), W139C, D601G, and L439R, wherein the amino acids are numbered with respect to a CoV S polypeptide having an amino acid sequence of SEQ ID NO: 2. In embodiments, the CoV S polypeptide contains one or more modifications selected from: K973P, V974P, an inactivated furin cleavage site, optionally wherein the inactivated furin cleavage site is QQAQ (SEQ ID NO: 7), W139C, S481P, and D601G wherein the amino acids are numbered with respect to a CoV S polypeptide having an amino acid sequence of SEQ ID NO: 2. In embodiments, the CoV S polypeptide containing one or more modifications selected from: K973P, V974P, an inactivated furin cleavage site, optionally wherein the inactivated furin cleavage site is QQAQ (SEQ ID NO: 7), W139C, S481P, D601G, and L439R has the amino acid sequence of SEQ ID NO: 153. In embodiments, the CoV S polypeptide having the amino acid sequence of SEQ ID NO: 153 comprises a signal peptide having an amino acid sequence of SEQ ID NO: 117. In embodiments, the CoV S polypeptide having the amino acid sequence of SEQ ID NO: 153 comprises a signal peptide having an amino acid sequence of SEQ ID NO: 5.

In embodiments, the CoV S polypeptide contains one or more modifications selected from: K973P, V974P, an inactivated furin cleavage site, optionally wherein the inactivated furin cleavage site is QQAQ (SEQ ID NO: 7), T82I, D240G, E471K, D601G, and A688V, wherein the amino acids are numbered with respect to a CoV S polypeptide having an amino acid sequence of SEQ ID NO: 2. In embodiments, the CoV S polypeptide containing one or more modifications selected from: K973P, V974P, an inactivated furin cleavage site, optionally wherein the inactivated furin cleavage site is QQAQ (SEQ ID NO: 7), T82I, D240G, E471K, D601G, and A688V, wherein the amino acids are numbered with respect to a CoV S polypeptide having an amino acid sequence of SEQ ID NO: 2, has an amino acid sequence of SEQ ID NO: 156. In embodiments, the CoV S polypeptide containing one or more modifications selected from: K973P, V974P, an inactivated furin cleavage site, optionally wherein the inactivated furin cleavage site is QQAQ (SEQ ID NO: 7), T82I, D240G, E471K, D601G, and A688V, wherein the amino acids are numbered with respect to a CoV S polypeptide having an amino acid sequence of SEQ ID NO: 2, comprises a signal peptide having an amino acid sequence of SEQ ID NO: 154 or SEQ ID NO: 5.

In embodiments, the CoV S polypeptide contains one or more modifications selected from: K973P, V974P, an inactivated furin cleavage site, optionally wherein the inactivated furin cleavage site is QQAQ (SEQ ID NO: 7), T82I, D240G, S464N, D601G, and A688V, wherein the amino acids are numbered with respect to a CoV S polypeptide having an amino acid sequence of SEQ ID NO: 2. In embodiments, the CoV S polypeptide containing one or more modifications selected from: K973P, V974P, an inactivated furin cleavage site, optionally wherein the inactivated furin cleavage site is QQAQ (SEQ ID NO: 7), T82I, D240G, S464N, D601G, and A688V, wherein the amino acids are numbered with respect to a CoV S polypeptide having an amino acid sequence of SEQ ID NO: 2, has an amino acid sequence of SEQ ID NO: 158. In embodiments, the CoV S polypeptide containing one or more modifications selected from: K973P, V974P, an inactivated furin cleavage site, optionally wherein the inactivated furin cleavage site is QQAQ (SEQ ID NO: 7), T82I, D240G, S464N, D601G, and A688V, wherein the amino acids are numbered with respect to a CoV S polypeptide having an amino acid sequence of SEQ ID NO: 2, comprises a signal peptide of SEQ ID NO: 154.

In embodiments, the CoV S polypeptide contains one or more modifications selected from: K973P, V974P, an inactivated furin cleavage site, optionally wherein the inactivated furin cleavage site is QQAQ (SEQ ID NO: 7), deletion of amino acid 56, deletion of amino acid 57, deletion of amino acid 131, a N488Y mutation, an A557D mutation, a D601G mutation, a P668H mutation, a T703I mutation, a S969A mutation, and a D1105H mutation, wherein the CoV S polypeptide is numbered with respect to the wild-type SARS-COV-2 S polypeptide having the amino acid sequence of SEQ ID NO: 2. In embodiments, the CoV S polypeptide contains one or more modifications selected from: K973P, V974P, an inactivated furin cleavage site, optionally wherein the inactivated furin cleavage site is QQAQ (SEQ ID NO: 7), deletion of amino acid 56, deletion of amino acid 57, deletion of amino acid 132, a N488Y mutation, an A557D mutation, a D601G mutation, a P668H mutation, a T7031 mutation, a S969A mutation, and a D1105H mutation, wherein the CoV S polypeptide is numbered with respect to the wild-type SARS-COV-2 S polypeptide having the amino acid sequence of SEQ ID NO: 2.

In embodiments, the CoV S polypeptide contains one or more modifications selected from: K973P, V974P, an inactivated furin cleavage site, optionally wherein the inactivated furin cleavage site is QQAQ (SEQ ID NO: 7), a D67A mutation, a L229H mutation, a R233I mutation, an A688V mutation, an N488Y mutation, a K404N mutation, a E471K mutation, and a D601G mutation, wherein the CoV S polypeptide is numbered with respect to the wild-type SARS-COV-2 S polypeptide having the amino acid sequence of SEQ ID NO: 2.

In embodiments, the CoV S polypeptide contains one or more modifications selected from: K973P, V974P, an inactivated furin cleavage site, optionally wherein the inactivated furin cleavage site is QQAQ (SEQ ID NO: 7), a L5F mutation, a T7N mutation, a P13S mutation, a D125Y mutation, a R177S mutation, a K404T mutation, a E471K mutation, a N488Y mutation, a D601G mutation, a H642Y mutation, a T1014I mutation, and a T1163F mutation, wherein the CoV S polypeptide is numbered with respect to the wild-type SARS-COV-2 S polypeptide having the amino acid sequence of SEQ ID NO: 2.

In embodiments, the CoV S polypeptide contains one or more modifications selected from: K986P, V987P, an inactivated furin cleavage site, optionally wherein the inactivated furin cleavage site is QQAQ (SEQ ID NO: 7), a S13I mutation, a W152C mutation, and a L452R mutation, wherein the CoV S polypeptide is numbered with respect to the wild-type SARS-COV-2 S polypeptide having the amino acid sequence of SEQ ID NO: 1. In embodiments, the CoV S polypeptide contains one or more modifications selected from: K986P, V987P, an inactivated furin cleavage site, optionally wherein the inactivated furin cleavage site is QQAQ (SEQ ID NO: 7), a S13I mutation, a W152C mutation, and a L452R mutation, wherein the CoV S polypeptide is numbered with respect to the wild-type SARS-COV-2 S polypeptide having the amino acid sequence of SEQ ID NO: 1 lacks an N-terminal signal peptide.

In embodiments, the CoV S polypeptide contains one or more modifications selected from: K986P, V987P, A67V, T95I, G142D, L212I, G339D, S371L, S373P, S375F, K417N, N440K, G446S, S477N, T478K, E484A, Q493R, G496S, Q498R, N501Y, Y505H, T547K, D614G, H655Y, H679K, P681H, N764K, D796Y, N856K, Q954H, N969K, L981F, deletion of amino acids 69, 70, 143, 144, 145, and 211, and insertion of the amino acids EPE between amino acids 214 and 215, optionally wherein the inactivated furin cleavage site is QQAQ (SEQ ID NO: 7), wherein the CoV S polypeptide is numbered with respect to the wild-type SARS-COV-2 S polypeptide having the amino acid sequence of SEQ ID NO: 1. In embodiments, the CoV S polypeptide having one or more of the aforementioned modifications lacks an N-terminal signal peptide. In embodiments, the CoV S polypeptide has an amino acid sequence of SEQ ID NO: 159.

In embodiments, the CoV S polypeptide contains one or more modifications selected from: K986P, V987P, A67V, T951, G142D, L212I, G339D, S371L, S373P, S375F, K417N, N440K, G446S, S477N, T478K, E484A, Q493R, G496S, Q498R, N501Y, Y505H, T547K, D614G, H655Y, H679K, P681H, N764K, D796Y, N856K, Q954H, N969K, L981F, optionally wherein the inactivated furin cleavage site is QQAQ (SEQ ID NO: 7), wherein the CoV S polypeptide is numbered with respect to the wild-type SARS-COV-2 S polypeptide having the amino acid sequence of SEQ ID NO: 1. In embodiments, the CoV S polypeptide having one or more of the aforementioned modifications lacks an N-terminal signal peptide. In embodiments, the CoV S polypeptide has an amino acid sequence of SEQ ID NO: 160.

In embodiments, the CoV S polypeptide is any one of SEQ ID NOS: 159 or 167. In embodiments, the amino acid sequence of the CoV S polypeptide has at least 90%, at least 95%, at least 96%, at least 97%, at least 98%, at least 99%, or at least 99.5% identity to any one of SEQ ID NOS: 159 or 167. In embodiments, the CoV S polypeptide is any one of SEQ ID NOS: 160 or 170. In embodiments, the amino acid sequence of the CoV S polypeptide has at least 90%, at least 95%, at least 96%, at least 97%, at least 98%, at least 99%, or at least 99.5% identity to any one of SEQ ID NOS: 160 or 170.

In embodiments, the CoV S polypeptide is encoded by a nucleic acid of any one of SEQ ID NOS: 161, 162, 163, 164, 165, 166, 168, 169, 171, and 172. In embodiments, the CoV S polypeptide of any one of SEQ ID NOS: 160, 170, 159, or 167 lacks the N-terminal signal peptide. For example, the CoV S polypeptide comprises the polypeptide sequence of SEQ ID NOS: 160, 170, 159, or 167, which is C-terminal to MFVFLVLLPLVSS (SEQ ID NO: 5).

In embodiments, the CoV S polypeptide contains a set of modifications as described in the table below, wherein the modifications are numbered with respect to SEQ ID NO: 1. In embodiments, the CoV S polypeptide contains a set of modifications as described in the table below; an inactivated furin cleavage site (optionally wherein the furin cleavage site is QQAQ (SEQ ID NO: 7)), and K986P and V987P modifications, wherein the modifications are numbered with respect to SEQ ID NO: 1.

In embodiments, the CoV S polypeptide contains one or more modifications at amino acids 180, 252, 253, 444, 478, and 521, wherein the CoV S polypeptide is numbered according to the CoV S polypeptide of SEQ ID NO: 1.

In embodiments, provided herein are CoV S polypeptides with modifications compared to a CoV S polypeptide having an amino acid sequence with at least 90%, at least 91%, at least 92%, at least 93%, at least 94%, at least 95%, at least 96%, at least 97%, at least 98%, at least 99%, or at least 100% identity to any one of SEQ ID NOS: 245, 250, 255, 260, 265, 269, 273, and 277. In embodiments, the modifications occur at one or more of amino acid positions 180, 252, 253, 444, 478, 486, 521, wherein the modifications are numbered according to a CoV S polypeptide having the amino acid sequence of SEQ ID NO: 1.

| | Modification | Modification | Modificatio | Modificatio | Modificatio | Modificatio |
|---|---|---|---|---|---|---|
| | Combination 1 | Combination 2 | Combination 3 | Combination 4 | Combination 5 | Combination 6 |
| Mutations | T19R | A67V | T19R | W64H | T19R | D80A |
| | T95I | T95I, G142D | G142D | H66W | G142D | D215G |
| | G142D | G339D | E156G | D213V | E156G | L242H |
| | Y145H | S371L | K417N | L214R | K417N | K417N |
| | E156G | S373P | N439K | T19R | L452R | E484K |
| | A222V | S375F | L452R | G142D | T478K | N501Y |
| | K417N | K417N, E484K | T478K | E156G | E484Q | D614G |
| | L452R | T478K, N501Y | E484K | K417N | D614G | A701V |
| | T478K | N440K, Q493K | N501Y | N439K | P681R | |
| | D614G | G446S, Q498R | D614G | L452R | D950N | |
| | P681R | Y505H, T547K | P681R | T478K | | |
| | D950N | D614G, H655Y | D950N | E484K | | |
| | | N679K, P681H | W258I | N501Y | | |
| | | N764K, D796Y | | D614G | | |
| | | N856J, Q954H | | P681R | | |
| | | N969K, L981 | | D950N | | |
| | | | | W258I | | |
| Deletions | F157 del | H69-V70 del | F157 del | F157 del | F157 del | |
| Insertions | R158 del | 143-145del | R158 del | R158 del | R158 del | |
| | | 211-212del | | | | |
| | | Insertion of | | | | |
| | | EPE between | | | | |
| | | 214 and 215 | | | | |

In embodiments, the CoV Spike(S) polypeptides comprise a polypeptide linker. In embodiments, the polypeptide linker contains glycine and serine. In embodiments, the linker has about 50%, about 55%, about 60%, about 65%, about 70%, about 75%, about 80%, about 85%, about 90%, about 95%, or about 100% glycine.

In embodiments, the polypeptide linker has a repeat of (SGGG)n (SEQ ID NO: 91), wherein n is an integer from 1 to 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50). In embodiments, the polypeptide linker has an amino acid sequence corresponding to SEQ ID NO: 90.

In embodiments, the polypeptide linker has a repeat of (GGGGS)n (SEQ ID NO: 93), wherein n is an integer from 1 to 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50).

In embodiments, the polypeptide linker has a repeat of (GGGS)n (SEQ ID NO: 92), wherein n is an integer from 1 to 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50).

In aspects, the polypeptide linker is a poly-(Gly)n linker, wherein n is 1, 2, 3, 4, 5, 6, 7, 8, 9, 10, 15, 16, 17, 18, 19, or 20. In other embodiments, the linker is selected from the group consisting of: dipeptides, tripeptides, and quadripeptides. In embodiments, the linker is a dipeptide selected from the group consisting of alanine-serine (AS), leucine-glutamic acid (LE), and serine-arginine (SR).

In embodiments, the polypeptide linker comprises between 1 to 100 contiguous amino acids of a naturally occurring CoV S polypeptide or of a CoV S polypeptide disclosed herein. In embodiments, the polypeptide linker has an amino acid sequence corresponding to SEQ ID NO: 94.

In embodiments, the CoV Spike(S) polypeptides comprise a foldon. In embodiments, the TMCT is replaced with a foldon. In embodiments, a foldon causes trimerization of the CoV Spike(S) polypeptide. In embodiments, the foldon is an amino acid sequence known in the art. In embodiments, the foldon has an amino acid sequence of SEQ ID NO: 68. In embodiments, the foldon is a T4 fibritin trimerization motif. In embodiments, the T4 fibritin trimerization domain has an amino acid sequence of SEQ ID NO: 103. In embodiments, the foldon is separated in amino acid sequence from the CoV Spike(S) polypeptide by a polypeptide linker. Non-limiting examples of polypeptide linkers are found throughout this disclosure.

In embodiments, the disclosure provides CoV S polypeptides comprising a fragment of a coronavirus S protein and nanoparticles and vaccines comprising the same. In embodiments, the fragment of the coronavirus S protein is between 10 and 1500 amino acids in length (e.g. about 10, about 20, about 30, about 40, about 50, about 60, about 70, about 80, about 90, about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, or about 1500 amino acids in length). In embodiments, the fragment of the coronavirus S protein is selected from the group consisting of the receptor binding domain (RBD), subdomain 1, subdomain 2, upper helix, fusion peptide, connecting region, heptad repeat 1, central helix, heptad repeat 2, NTD, and TMCT.

In embodiments, the CoV S polypeptide comprises an RBD and a subdomain 1. In embodiments, the CoV S polypeptide comprising an RBD and a subdomain 1 is amino acids 319 to 591 of SEQ ID NO: 1.

In embodiments, the CoV S polypeptide contains a fragment of a coronavirus S protein, wherein the fragment of the coronavirus S protein is the RBD. Non-limiting examples of RBDs include the RBD of SARS-COV-2 (amino acid sequence=SEQ ID NO: 69), the RBD of SARS (amino acid sequence=SEQ ID NO: 70), and the RBD of MERS, (amino acid sequence=SEQ ID NO: 71).

In embodiments, the CoV S polypeptide contains two or more RBDs, which are connected by a polypeptide linker. In embodiments, the polypeptide linker has an amino acid sequence of SEQ ID NO: 90 or SEQ ID NO: 94.

In embodiments, the CoV S polypeptide contains 1, 2, 3, 4, 5, 6, 7, 8, 9, 10, 11, 12, 13, 14, 15, 16, 17, 18, 19, or 20 RBDs.

In some embodiments, the CoV S polypeptide contains two or more SARS-COV-2 RBDs, which are connected by a polypeptide linker. In embodiments, the antigen containing two or more SARS-COV-2 RBDs has an amino acid sequence corresponding to one of SEQ ID NOS: 72-75.

In embodiments, the CoV S polypeptide contains a SARS-COV-2 RBD and a SARS RBD. In embodiments, the CoV S polypeptide comprises a SARS-COV-2 RBD and a SARS RBD, wherein each RBD is separated by a polypeptide linker. In embodiments, the CoV S polypeptide comprising a SARS-COV-2 RBD and a SARS RBD has an amino acid sequence selected from the group consisting of SEQ ID NOS: 76-79.

In embodiments, the CoV S polypeptide contains a SARS-COV-2 RBD and a MERS RBD. In embodiments, the CoV S polypeptide comprises a SARS-COV-2 RBD and a MERS RBD, wherein each RBD is separated by a polypeptide linker.

In embodiments, the CoV S polypeptide comprises a SARS RBD and a MERS RBD. In embodiments, the CoV S polypeptide comprises a SARS RBD and a MERS RBD, wherein each RBD is separated by a polypeptide linker.

In embodiments, the CoV S polypeptide contains a SARS-COV-2 RBD, a SARS RBD, and a MERS RBD. In embodiments, the CoV S polypeptide contains a SARS-COV-2 RBD, a SARS

RBD, and a MERS RBD, wherein each RBD is separated by a polypeptide linker. In embodiments, the CoV S polypeptide comprising a SARS-COV-2 RBD, a SARS RBD, and a MERS RBD has an amino acid sequence selected from the group consisting of SEQ ID NOS: 80-83.

In embodiments, the CoV S polypeptides described herein are expressed with an N-terminal signal peptide. In embodiments, the N-terminal signal peptide has an amino acid sequence of SEQ ID NO: 5 (MFVFLVLLPLVSS). In embodiments, the N-terminal signal peptide has an amino acid sequence of SEQ ID NO: 117 (MFVFLVLLPLVSI). In embodiments, the N-terminal signal peptide has an amino acid sequence of SEQ ID NO: 154 (MFVFFVLLPLVSS). In embodiments, the N-terminal signal peptide has an amino acid sequence of SEQ ID NO: 193 (MFGFLVLLPLVSS). In embodiments, the signal peptide may be replaced with any signal peptide that enables expression of the CoV S protein. In embodiments, one or more of the CoV S protein signal peptide amino acids may be deleted or mutated. An initiating methionine residue is maintained to initiate expression. In embodiments, the CoV S polypeptides are encoded by a nucleic acid sequence selected from the group consisting of SEQ ID NO: 35, SEQ ID NO: 37, SEQ ID NO: 95, SEQ ID NO: 43, SEQ ID NO: 47, SEQ ID NO: 50, SEQ ID NO: 53, SEQ ID NO: 55, SEQ ID NO: 57, SEQ ID NO: 96, SEQ ID NO: 60, SEQ ID NO: 131, SEQ ID NO: 135, SEQ ID NO: 142, SEQ ID NO: 145, SEQ ID NO: 148, SEQ ID NO: 150, SEQ ID NO: 196, SEQ ID NO: 197; SEQ ID NO: 198; SEQ ID NO: 199; SEQ ID NO: 201; SEQ ID NO: 202; SEQ ID NO: 204; SEQ ID NO: 206; SEQ ID NO: 208; SEQ ID NO: 210; SEQ ID NO: 212; SEQ ID NO: 214; SEQ ID NO: 216; SEQ ID NO: 329; SEQ ID NO: 330; SEQ ID NO: 331; SEQ ID NO: 332; SEQ ID NO: 333; and SEQ ID NO: 334. In embodiments, the N-terminal signal peptide of the CoV S polypeptide contains a mutation at Ser-13 relative to the native CoV Spike(S) signal polypeptide (SEQ ID NO: 5). In embodiments, Ser-13 is mutated to any natural amino acid. In embodiments, Ser-13 is mutated to alanine, methionine, isoleucine, leucine, threonine, or valine. In embodiments, Ser-13 is mutated to isoleucine.

Following expression of the CoV S protein in a host cell, the N-terminal signal peptide is cleaved to provide the mature CoV protein sequence (SEQ ID NOS: 2, 4, 38, 41, 44, 48, 51, 54, 58, 61, 63, 65, 67, 73, 75, 78, 79, 82, 83, 85, 87, 89,106,110, 132, 133, 114, 138, 141, 144, 147, 151, 153, 156, 158, 174, 175, 176, 181-184, 186, 188, 190, 195, 217-228 233-236, 243, 255-264, 273-280, 283-284, 287-288, 291-292, 294, 296, 298, 300, 302, 304, 306, 308, 310, 312, 314, 316, 318, 320, 322, 324, 236, and 328). In embodiments, the signal peptide is cleaved by host cell proteases. In aspects, the full-length protein may be isolated from the host cell and the signal peptide cleaved subsequently.

Following cleavage of the signal peptide from a CoV Spike(S) polypeptide with an amino acid sequence corresponding to any one of SEQ ID NOS: 1, 3, 36, 40, 42, 46, 49, 52, 56, 59, 62, 64, 66, 72, 74, 76, 77, 80, 81, 84, 86, 87, 105, 107, 88, 109,130, 134, 136, 137, 140, 143, 146, 149, 152, 155, 157, 159, 160, 173, 177-180, 185, 189, 191, 194, 200, 203, 205, 207, 209, 211, 213, 215, 229-232, 242, 245-254, 265-272, 281-282, 285-286, 289-290, and 293, 295, 297, 299, 301, 303, 305, 307, 309, 311, 313, 315, 317, 319, 321, 323, 325, 327, 329, 330, 331, 332, 333, and 334 during expression and purification, a mature polypeptide having an amino acid sequence selected from the group consisting of SEQ ID NOS: 2, 4, 38, 41, 44, 48, 51, 54, 58, 61, 63, 65, 67, 73, 75, 78, 79, 82, 83, 85, 106, 108, 89, and 110, 112-115, 132, 133, 114, 138, 141, 144, 147, 151, 153, 156, 158, 174, 175, 176, 181-184, 186, 188, 190, 195, 217-228, 233-236, 243, 255-264, 273-280, 283, 284, 287, 288, 291, 292, and 294, 296, 298, 300, 302, 304, 306, 308, 310, 312, 314, 316, 318, 320, 322, 324, 236, and 328 is obtained and used to produce a CoV S nanoparticle vaccine or CoV S nanoparticles.

Advantageously, the disclosed CoV S polypeptides may have enhanced protein expression and stability relative to the native CoV Spike(S) protein.

In embodiments, the CoV S polypeptides described herein contain further modifications from the native coronavirus S protein (SEQ ID NO: 2). In embodiments, the coronavirus S proteins described herein exhibit at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity to the native coronavirus S protein. A person of skill in the art would use known techniques to calculate the percent identity of the recombinant coronavirus S protein to the native protein or to any of the CoV S polypeptides described herein. For example, percentage identity can be calculated using the tool CLUSTALW2, which is available online. The following default parameters may be used for CLUSTALW2 Pairwise alignment: Protein Weight Matrix=Gonnet; Gap Open=10; Gap Extension=0.1.

In embodiments, the CoV S polypeptides described herein are at least 90%, at least 91%, at least 92%, at least 93%, at least 94%, at least 95%, at least 96%, at least 97%, at least 98%, at least 99%, or at least 99.5% identical to the CoV S polypeptide having an amino acid sequence of any one of SEQ ID NO: 87, SEQ ID NO: 174, SEQ ID NO: 175, SEQ ID NO: 176, SEQ ID NOS: 181-184, SEQ ID NO: 186, SEQ ID NO: 188, SEQ ID NO: 190, SEQ ID NO: 195; SEQ ID NOS: 217-228, SEQ ID NOS: 233-236, and SEQ ID NO: 243, SEQ ID NOS 255-328; SEQ ID NOS: 329-333; SEQ ID NO: 334. A CoV S polypeptide may have a deletion, an insertion, or mutation of up to about 1, up to about 2, up to about 3, up to about 4, up to about 5, up to about 10, up to about 15, up to about 20, up to about 25, up to about 30, up to about 35, up to about 40, up to about 45, or up to about 50 amino acids compared to the amino acid sequence of the CoV S polypeptide having an amino acid sequence of any one of SEQ ID NO: 87, SEQ ID NO: 174, SEQ ID NO: 175, SEQ ID NO: 176, SEQ ID NOS: 181-184, SEQ ID NO: 186, SEQ ID NO: 188, SEQ ID NO: 190, SEQ ID NO: 195; SEQ ID NOS: 217-228, SEQ ID NOS: 233-236, SEQ ID NO: 243, and SEQ ID NOS: 255-328; SEQ ID NOS: 329-333; SEQ ID NO: 334. A CoV S polypeptide may have may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, between about 25 and 30 amino acids, between about 30 and 35 amino acids, between about 35 and 40 amino acids, between about 40 and 45 amino acids, or between about 45 and 50 amino acids, as compared to the CoV S polypeptide having an amino acid sequence of any one of SEQ ID NO: 87, SEQ ID NO: 174, SEQ ID NO: 175, SEQ ID NO: 176, SEQ ID NOS: 181-184, SEQ ID NO: 186, SEQ ID NO: 188, SEQ ID NO: 190, SEQ ID NO: 195; SEQ ID NOS: 217-228, SEQ ID NOS: 233-236, SEQ ID NO: 243, and SEQ ID NOS: 255-302; SEQ ID NOS: 329-333; SEQ ID NO: 334. In embodiments, the CoV S polypeptides described herein comprise about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, or about 75 deletions, insertions, or mutations compared to the coronavirus S protein having an amino acid sequence of any one of SEQ ID NO: 87, SEQ ID NO: 174, SEQ ID NO: 175, SEQ ID NO: 176, SEQ ID NOS: 181-184, SEQ ID NO: 186, SEQ ID NO: 188, SEQ ID NO: 190, SEQ ID NO: 195; SEQ ID NOS: 217-228, SEQ ID NOS: 233-236, SEQ ID NO: 243, SEQ ID NOS: 255-328; SEQ ID NOS: 329-333; SEQ ID NO: 334.

In embodiments, the coronavirus S polypeptide is extended at the N-terminus, the C-terminus, or both the N-terminus and the C-terminus. In aspects, the extension is a tag useful for a function, such as purification or detection. In aspects the tag contains an epitope. For example, the tag may be a polyglutamate tag, a FLAG-tag, a HA-tag, a polyHis-tag (having about 5-10 histidines) (SEQ ID NO: 101), a hexahistidine tag (SEQ ID NO: 100), an 8×-His-tag (having eight histidines) (SEQ ID NO: 102), a Myc-tag, a Glutathione-S-transferase-tag, a Green fluorescent protein-tag, Maltose binding protein-tag, a Thioredoxin-tag, or an Fc-tag. In other aspects, the extension may be an N-terminal signal peptide fused to the protein to enhance expression. While such signal peptides are often cleaved during expression in the cell, some nanoparticles may contain the antigen with an intact signal peptide. Thus, when a nanoparticle comprises an antigen, the antigen may contain an extension and thus may be a fusion protein when incorporated into nanoparticles. For the purposes of calculating identity to the sequence, extensions are not included. In embodiments, the tag is a protease cleavage site. Non-limiting examples of protease cleavage sites include the HRV3C protease cleavage site, chymotrypsin, trypsin, elastase, endopeptidase, caspase-1, caspase-2, caspase-3, caspase-4, caspase-5, caspase-6, caspase-7, caspase-8, caspase-9, caspase-10, enterokinase, factor Xa, Granzyme B, TEV protease, and thrombin. In embodiments, the protease cleavage site is an HRV3C protease cleavage site. In embodiments, the protease cleavage site comprises an amino acid sequence of SEQ ID NO: 98.

In embodiments, the CoV S glycoprotein comprises a fusion protein. In embodiments, the CoV S glycoprotein comprises an N-terminal fusion protein. In embodiments, the Cov S glycoprotein comprises a C-terminal fusion protein. In embodiments, the fusion protein encompasses a tag useful for protein expression, purification, or detection. In embodiments, the tag is a polyHis-tag (having about 5-10 histidines), a Myc-tag, a Glutathione-S-transferase-tag, a Green fluorescent protein-tag, Maltose binding protein-tag, a Thioredoxin-tag, a Strep-tag, a Twin-Strep-tag, or an Fc-tag. In embodiments, the tag is an Fc-tag. In embodiments, the Fc-tag is monomeric, dimeric, or trimeric. In embodiments, the tag is a hexahistidine tag, e.g. a polyHis-tag which contains six histidines (SEQ ID NO: 100). In embodiments, the tag is a Twin-Strep-tag with an amino acid sequence of SEQ ID NO: 99.

In embodiments, the CoV S polypeptide is a fusion protein comprising another coronavirus protein. In embodiments, the other coronavirus protein is from the same coronavirus. In embodiments, the other coronavirus protein is from a different coronavirus.

In aspects, the CoV S protein may be truncated. For example, the N-terminus may be truncated by about 10 amino acids, about 30 amino acids, about 50 amino acids, about 75 amino acids, about 100 amino acids, or about 200 amino acids. The C-terminus may be truncated instead of or in addition to the N-terminus. For example, the C-terminus may be truncated by about 10 amino acids, about 30 amino acids, about 50 amino acids, about 75 amino acids, about 100 amino acids, or about 200 amino acids. For purposes of calculating identity to the protein having truncations, identity is measured over the remaining portion of the protein.

### Nanoparticles Containing CoV Spike(S) Polypeptides

In embodiments, the mature CoV S polypeptide antigens are used to produce a vaccine comprising coronavirus S nanoparticles. In embodiments, nanoparticles of the present disclosure comprise the CoV S polypeptides described herein. In embodiments, the nanoparticles of the present disclosure comprise CoV S polypeptides associated with a detergent core. The presence of the detergent facilitates formation of the nanoparticles by forming a core that organizes and presents the antigens. In embodiments, the nanoparticles may contain the CoV S polypeptides assembled into multi-oligomeric glycoprotein-detergent (e.g. PS80) nanoparticles with the head regions projecting outward and hydrophobic regions and PS80 detergent forming a central core surrounded by the glycoprotein. In embodiments, the CoV S polypeptide inherently contains or is adapted to contain a transmembrane domain to promote association of the protein into a detergent core. In embodiments, the CoV S polypeptide contains a head domain. Primarily the transmembrane domains of a CoV S polypeptide trimer associate with detergent; however, other portions of the polypeptide may also interact. Advantageously, the nanoparticles have improved resistance to environmental stresses such that they provide enhanced stability and/or improved presentation to the immune system due to organization of multiple copies of the protein around the detergent.

In embodiments, the detergent core is a non-ionic detergent core. In embodiments, the CoV S polypeptide is associated with the non-ionic detergent core. In embodiments, the detergent is selected from the group consisting of polysorbate-20 (PS20), polysorbate-40 (PS40), polysorbate-60 (PS60), polysorbate-65 (PS65) and polysorbate-80 (PS80).

In embodiments, the detergent is PS80.

In embodiments, the CoV S polypeptide forms a trimer. In embodiments, the CoV S polypeptide nanoparticles are composed of multiple polypeptide trimers surrounding a non-ionic detergent core. In embodiments, the nanoparticles contain at least about 1 trimer or more. In embodiments, the nanoparticles contain at least about 5 trimers to about 30 trimers of the Spike protein. In embodiments, each nanoparticle may contain 1, 2, 3, 4, 5, 6, 7, 8, 9, 10, 11, 12, or 15, 20, 25, or 30 trimers, including all values and ranges in between. Compositions disclosed herein may contain nanoparticles having different numbers of trimers. For example, a composition may contain nanoparticles where the number of trimers ranges from 2-9; in embodiments, the nanoparticles in a composition may contain from 2-6 trimers. In embodiments, the compositions contain a heterogeneous population of nanoparticles having 2 to 6 trimers per nanoparticle, or 2 to 9 trimers per nanoparticle. In embodiments, the compositions may contain a substantially homogenous population of nanoparticles. For example, the population may contain about 95% nanoparticles having 5 trimers.

In embodiments, provided herein are nanoparticles having from 1 to 50 CoV S polypeptides, wherein the amino acid sequence of each CoV S polypeptide is different. In embodiments, the nanoparticles comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 different CoV S polypeptides.

The nanoparticles disclosed herein range in particle size. In embodiments, the nanoparticles disclosed herein range in particle size from a Z-ave size from about 20 nm to about 60 nm, about 20 nm to about 50 nm, about 20 nm to about 45 nm, about 20 nm to about 35 nm, about 20 nm to about 30 nm, about 25 nm to about 35 nm, about 25 nm to about 45 nm, about 30 nm to about 120 nm, about 30 nm to about 80 nm, about 30 nm to about 60 nm, about 30 nm to about 65 nm, or from about 30 nm to about 50 nm. Particle size (Z-ave) is measured by dynamic light scattering (DLS) using a Zetasizer NanoZS (Malvern, UK), unless otherwise specified.

In embodiments, the nanoparticles comprising the CoV S polypeptides disclosed herein have a reduced particle size compared to nanoparticles comprising a wild-type CoV S polypeptide. In embodiments, the CoV S polypeptides are at least about 40% smaller in particle size, for example, at least about 40%, at least about 45%, at least about 50%, at least about 55%, at least about 60%, at least about 65%, at least about 70%, at least about 75%, at least about 80%, or at least about 85% smaller in particle size.

The nanoparticles comprising CoV S polypeptides disclosed herein are more homogenous in size, shape, and mass than nanoparticles comprising a wild-type CoV S polypeptide. The polydispersity index (PDI), which is a measure of heterogeneity, is measured by dynamic light scattering using a Malvern Setasizer unless otherwise specified. In embodiments, the particles measured herein have a PDI from about 0.1 to about 0.45, for example, about 0.1, about 0.2, about 0.25, about 0.29, about 0.3, about 0.35, about 0.40, or about 0.45. In embodiments, the nanoparticles measured herein have a PDI that is at least about 25% smaller than the PDI of nanoparticles comprising the wild-type CoV S polypeptide of SEQ ID NO: 2, for example, at least about 25%, at least about 30%, at least about 35%, at least about 40%, at least about 45%, at least about 50%, at least about 55%, or at least about 60%, smaller.

The CoV S polypeptides and nanoparticles comprising the same have improved thermal stability as compared to the wild-type CoV S polypeptide or a nanoparticle thereof. The thermal stability of the CoV S polypeptides is measured using differential scanning calorimetry (DSC) unless otherwise specified. The enthalpy of transition (ΔHcal) is the energy required to unfold a CoV S polypeptide. In embodiments, the CoV S polypeptides have an increased ΔHcal as compared to the wild-type CoV S polypeptide. In embodiments, the ΔHcal of a CoV S polypeptide is about 2-fold, about 3-fold, about 4-fold, about 5-fold, about 6-fold, about 7-fold, about 8-fold, about 9-fold, or about 10-fold greater than the ΔHcal of a wild-type CoV S polypeptide.

Several nanoparticle types may be included in vaccine compositions disclosed herein. In aspects, the nanoparticle type is in the form of an anisotropic rod, which may be a dimer or a monomer. In other aspects, the nanoparticle type is a spherical oligomer. In yet other aspects, the nanoparticle may be described as an intermediate nanoparticle, having sedimentation properties intermediate between the first two types. Formation of nanoparticle types may be regulated by controlling detergent and protein concentration during the production process. Nanoparticle type may be determined by measuring sedimentation co-efficient.

### Production of Nanoparticles Containing CoV S Polypeptide Antigens

The nanoparticles of the present disclosure are non-naturally occurring products, the components of which do not occur together in nature. Generally, the methods disclosed herein use a detergent exchange approach wherein a first detergent is used to isolate a protein and then that first detergent is exchanged for a second detergent to form the nanoparticles.

The antigens contained in the nanoparticles are typically produced by recombinant expression in host cells. Standard recombinant techniques may be used. In embodiments, the CoV S polypeptides are expressed in insect host cells using a baculovirus system. In embodiments, from 1-50 CoV S polypeptides are co-expressed in a host cell. In embodiments, the baculovirus is a cathepsin-L knock-out baculovirus, a chitinase knock-out baculovirus. Optionally, the baculovirus is a double knock-out for both cathepsin-L and chitinase. High level expression may be obtained in insect cell expression systems. Non limiting examples of insect cells are, Spodoptera frugiperda (Sf) cells, e.g. Sf9, Sf21, Trichoplusiani cells, e.g. High Five cells, and Drosophila S2 cells. In embodiments, the CoV S polypeptide described herein are produced in any suitable host cell. In embodiments, the host cell is an insect cell. In embodiments, the insect cell is an Sf9 cell.

Typical transfection and cell growth methods can be used to culture the cells. Vectors, e.g., vectors comprising polynucleotides that encode fusion proteins, can be transfected into host cells according to methods well known in the art. For example, introducing nucleic acids into eukaryotic cells can be achieved by calcium phosphate co-precipitation, electroporation, microinjection, lipofection, and transfection employing polyamine transfection reagents. In one embodiment, the vector is a recombinant baculovirus.

Methods to grow host cells include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag®, Wave Biotech, Bridgewater, N.J.). In other embodiment, the pre-sterilized plastic bags are about 50 L to 3500 L bags.

### Extraction and Purification of Nanoparticles Containing CoV Spike(S) Protein Antigens

After growth of the host cells, the protein may be harvested from the host cells using detergents and purification protocols. In embodiments, multiple CoV S proteins are purified simultaneously. In embodiments, host cells expressing multiple CoV S proteins arc pooled together. Once the host cells have grown for 48 to 96 hours, the cells are isolated from the media and a detergent-containing solution is added to solubilize the cell membrane, releasing the protein in a detergent extract. Triton X-100 and TERGITOL® nonylphenol ethoxylate, also known as NP-9, are each preferred detergents for extraction. The detergent may be added to a final concentration of about 0.1% to about 1.0%. For example, the concentration may be about 0.1%, about 0.2%, about 0.3%, about 0.5%, about 0.7%, about 0.8%, or about 1.0%. The range may be about 0.1% to about 0.3%. In aspects, the concentration is about 0.5%.

In other aspects, different first detergents may be used to isolate the protein from the host cell. For example, the first detergent may be Bis (polyethylene glycol bis[imidazoylcarbonyl]), nonoxynol-9, Bis (polyethylene glycol bis[imidazoyl carbonyl]), BRIJ® Polyethylene glycol dodecyl ether 35, BRIJ® Polyethylene glycol (3) cetyl ether 56, BRIJ® alcohol ethoxylate 72, BRIJ® Polyoxyl 2 stearyl ether 76, BRIJ® polyethylene glycol monoolelyl ether 92V, BRIJ® Polyoxyethylene (10) oleyl ether 97, BRIJ® Polyethylene glycol hexadecyl ether 58P, CREMOPHOR® EL Macrogolglycerol ricinoleate, Decaethyleneglycol monododecyl ether, N-Decanoyl-N-methylglucamine, n-Decyl alpha-Dglucopyranoside, Decyl beta-D-maltopyranoside, n-Dodecanoyl-N-methylglucamide, nDodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O-(N-heptylcarbamoyl)-alpha-D-glucopyranoside, Nonaethylene glycol monododecyl ether, N-Nonanoyl-N-methylglucamine, N-Nonanoyl N-methylglucamine, Octaethylene glycol monodecyl ether, Octaethylene glycolmonododecyl ether, Octaethylene glycol monohexadecyl ether, Octaethylene glycol monooctadecyl ether, Octaethylene glycol monotetradecyl ether, Octyl-beta-D glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis (imidazolyl carbonyl), Polyoxyethylene 25 propylene glycol stearate, Saponin from Quillaja bark, SPAN® 20 sorbitan laurate, SPAN® 40 sorbitan monopalmitate, SPAN® 60 sorbitan stearate, SPAN® 65 sorbitan tristearate, SPAN® 80 sorbitane monooleate, SPAN® 85 sorbitane trioleate, TERGITOL® secondary alcohol ethoxylate Type 15-S-12, TERGITOL® secondary alcohol ethoxylate Type 15-S-30, TERGITOL® secondary alcohol ethoxylate Type 15-S-5, TERGITOL® secondary alcohol ethoxylate Type 15-S-7, TERGITOL® secondary alcohol ethoxylate Type 15-S-9, TERGITOL® nonylphenol ethoxylate Type NP-10, TERGITOL® nonylphenol ethoxylate Type NP-4, TERGITOL® nonylphenol ethoxylate Type NP-40, TERGITOL® nonylphenol ethoxylate Type NP-7, TERGITOL® nonylphenol ethoxylate Type NP-9, TERGITOL® branched secondary alcohol ethoxylate Type TMN-10, TERGITOL® branched secondary alcohol ethoxylate Type TMN-6, TRITON™ X-100 Polyethylene glycol tert-octylphenyl ether or combinations thereof.

The nanoparticles may then be isolated from cellular debris using centrifugation. In embodiments, gradient centrifugation, such as using cesium chloride, sucrose and iodixanol, may be used. Other techniques may be used as alternatives or in addition, such as standard purification techniques including, e.g., ion exchange, affinity, and gel filtration chromatography.

For example, the first column may be an ion exchange chromatography resin, such as FRACTOGEL® EMD methacrylate based polymeric beads TMAE (EMD Millipore), the second column may be a lentil (Lens culinaris) lectin affinity resin, and the third column may be a cation exchange column such as a FRACTOGEL® EMD methacrylate based polymeric beads SO3 (EMD Millipore) resin. In other aspects, the cation exchange column may be an MMC column or a Nuvia C Prime column (Bio-Rad Laboratories, Inc). Preferably, the methods disclosed herein do not use a detergent extraction column; for example a hydrophobic interaction column. Such a column is often used to remove detergents during purification but may negatively impact the methods disclosed here.

### Detergent Exchange of Nanoparticles Containing CoV S Polypeptide Antigens

To form nanoparticles, the first detergent, used to extract the protein from the host cell is substantially replaced with a second detergent to arrive at the nanoparticle structure. NP-9 is a preferred extraction detergent. Typically, the nanoparticles do not contain detectable NP-9 when measured by HPLC. The second detergent is typically selected from the group consisting of PS20, PS40, PS60, PS65, and PS80. Preferably, the second detergent is PS80.

In particular aspects, detergent exchange is performed using affinity chromatography to bind glycoproteins via their carbohydrate moiety. For example, the affinity chromatography may use a legume lectin column. Legume lectins are proteins originally identified in plants and found to interact specifically and reversibly with carbohydrate residues. See, for example, Sharon and Lis, “Legume lectins--a large family of homologous proteins,” FASEB J. 1990 November; 4 (14): 3198-208; Liener, “The Lectins: Properties, Functions, and Applications in Biology and Medicine,” Elsevier, 2012. Suitable lectins include concanavalin A (con A), pea lectin, sainfoin lect, and lentil lectin. Lentil lectin is a preferred column for detergent exchange due to its binding properties. Lectin columns are commercially available; for example, Capto Lentil Lectin, is available from GE Healthcare. In certain aspects, the lentil lectin column may use a recombinant lectin. At the molecular level, it is thought that the carbohydrate moieties bind to the lentil lectin, freeing the amino acids of the protein to coalesce around the detergent resulting in the formation of a detergent core providing nanoparticles having multiple copies of the antigen, e.g., glycoprotein oligomers which can be dimers, trimers, or tetramers anchored in the detergent. In embodiments, the CoV S polypeptides form trimers. In embodiments, the CoV S polypeptide trimers are anchored in detergent. In embodiments, each CoV S polypeptide nanoparticle contains at least one trimer associated with a non-ionic core.

The detergent, when incubated with the protein to form the nanoparticles during detergent exchange, may be present at up to about 0.1% (w/v) during early purifications steps and this amount is lowered to achieve the final nanoparticles having optimum stability. For example, the non-ionic detergent (e.g., PS80) may be about 0.005% (v/v) to about 0.1% (v/v), for example, about 0.005% (v/v), about 0.006% (v/v), about 0.007% (v/v), about 0.008% (v/v), about 0.009% (v/v), about 0.01% (v/v), about 0.015% (v/v), about 0.02% (v/v), about 0.025% (v/v), about 0.03% (v/v), about 0.035% (v/v), about 0.04% (v/v), about 0.045% (v/v), about 0.05% (v/v), about 0.055% (v/v), about 0.06% (v/v), about 0.065% (v/v), about 0.07% (v/v), about 0.075% (v/v), about 0.08% (v/v), about 0.085% (v/v), about 0.09% (v/v), about 0.095% (v/v), or about 0.1% (v/v) PS80. In embodiments, the nanoparticle contains about 0.03% to about 0.05% PS80. In embodiments, the nanoparticle contains about 0.01% (v/v) PS80.

In embodiments, purified CoV S polypeptides are dialyzed. In embodiments, dialysis occurs after purification. In embodiments, the CoV S polypeptides are dialyzed in a solution comprising sodium phosphate, NaCl, and PS80. In embodiments, the dialysis solution comprising sodium phosphate contains between about 5 mM and about 100 mM of sodium phosphate, for example, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM sodium phosphate. In embodiments, the pH of the solution comprising sodium phosphate is about 6.5, about 6.6, about 6.7, about 6.8, about 6.9, about 7.0, about 7.1, about 7.2, about 7.3, about 7.4, or about 7.5. In embodiments, the dialysis solution comprising sodium chloride comprises about 50 mM NaCl to about 500 mM NaCl, for example, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM NaCl. In embodiments, the dialysis solution comprising PS80 comprises about 0.005% (v/v), about 0.006% (v/v), about 0.007% (v/v), about 0.008% (v/v), about 0.009% (v/v), about 0.01% (v/v), about 0.015% (v/v), about 0.02% (v/v), about 0.025% (v/v), about 0.03% (v/v), about 0.035% (v/v), about 0.04% (v/v), about 0.045% (v/v), about 0.05% (v/v), about 0.055% (v/v), about 0.06% (v/v), about 0.065% (v/v), about 0.07% (v/v), about 0.075% (v/v), about 0.08% (v/v), about 0.085% (v/v), about 0.09% (v/v), about 0.095% (v/v), or about 0.1% (v/v) PS80. In embodiments, the dialysis solution comprises about 25 mM sodium phosphate (pH 7.2), about 300 mM NaCl, and about 0.01% (v/v) PS80.

Detergent exchange may be performed with proteins purified as discussed above and purified, frozen for storage, and then thawed for detergent exchange.

Stability of compositions disclosed herein may be measured in a variety of ways. In one approach, a peptide map may be prepared to determine the integrity of the antigen protein after various treatments designed to stress the nanoparticles by mimicking harsh storage conditions. Thus, a measure of stability is the relative abundance of antigen peptides in a stressed sample compared to a control sample. For example, the stability of nanoparticles containing the CoV S polypeptides may be evaluated by exposing the nanoparticles to various pHs, proteases, salt, oxidizing agents, including but not limited to hydrogen peroxide, various temperatures, freeze/thaw cycles, and agitation. It is thought that the position of the glycoprotein anchored into the detergent core provides enhanced stability by reducing undesirable interactions. For example, the improved protection against protease-based degradation may be achieved through a shielding effect whereby anchoring the glycoproteins into the core at the molar ratios disclosed herein results in steric hindrance blocking protease access. Stability may also be measured by monitoring intact proteins.

### Immunogenic Compositions Containing CoV S Polypeptide Antigens

In embodiments, provided herein are immunogenic compositions comprising from 1 to 50, from 2 to 50, from 3 to 50, from 4 to 50, from 5 to 50, from 1 to 25, from 2 to 25, from 3 to 25, from 4 to 25, from 5 to 25, from 1 to 10, from 2 to 10, from 3 to 10, from 4 to 10, from 5 to 10, of from 3 to 8 CoV S glycoproteins. In embodiments, provided herein are immunogenic compositions comprising five different CoV S glycoproteins. In embodiments, the CoV S glycoproteins are in the form of nanoparticles. In embodiments, a nanoparticle comprises from 1 to 50, from 2 to 50, from 3 to 50, from 4 to 50, from 5 to 50, from 1 to 25, from 2 to 25, from 3 to 25, from 4 to 25, from 5 to 25, from 1 to 10, from 2 to 10, from 3 to 10, from 4 to 10, from 5 to 10, of from 3 to 8 CoV S glycoproteins. In embodiments, a nanoparticle comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 CoV S glycoproteins.

In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 260. In embodiments, the immunogenic composition further comprises from 1-10 additional CoV S glycoproteins. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 261. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 262. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 263. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 264. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 87. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of any one of SEQ ID NOS: 244-328.

In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 274. In embodiments, the immunogenic composition further comprises from 1-10 additional CoV S glycoproteins. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 276. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 278. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 280. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 87. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of any one of SEQ ID NOS: 244-328.

In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 284. In embodiments, the immunogenic composition further comprises from 1-10 additional CoV S glycoproteins. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 288. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 292. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 87. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of any one of SEQ ID NOS: 244-328.

In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 260. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 274. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 222. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 87 and a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 274. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 87 and a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of SEQ ID NO: 222. In embodiments, the immunogenic composition comprises a CoV S glycoprotein with at least 90%, a least 91%, at least 92%, at least 93%, at least 94%, at least 95%, at least 96%, at least 97%, at least 98%, at least 99%, or 100% identity to a CoV S glycoprotein of any one of SEQ ID NOS: 244-328.

In embodiments, the immunogenic compositions are vaccine compositions. In aspects, the immunogenic composition may contain nanoparticles with antigens from more than one viral strain from the same species of virus. In another embodiment, the disclosures provide for a pharmaceutical pack or kit comprising one or more containers filled with one or more of the components of the immunogenic compositions.

Compositions disclosed herein may be used either prophylactically or therapeutically, but will typically be prophylactic. Accordingly, the disclosure includes methods for treating or preventing infection. The methods involve administering to the subject a therapeutic or prophylactic amount of the immunogenic compositions of the disclosure. Preferably, the pharmaceutical composition is a vaccine composition that provides a protective effect. In other aspects, the protective effect may include amelioration of a symptom associated with infection in a percentage of the exposed population. For example, the composition may prevent or reduce one or more virus disease symptoms selected from: fever fatigue, muscle pain, headache, sore throat, vomiting, diarrhea, rash, symptoms of impaired kidney and liver function, internal bleeding and external bleeding, compared to an untreated subject.

The nanoparticles may be formulated for administration as vaccines in the presence of various excipients, buffers, and the like. For example, the vaccine compositions may contain sodium phosphate, sodium chloride, and/or histidine. Sodium phosphate may be present at about 10 mM to about 50 mM, about 15 mM to about 25 mM, or about 25 mM; in particular cases, about 22 mM sodium phosphate is present. Histidine may be present about 0.1% (w/v), about 0.5% (w/v), about 0.7% (w/v), about 1% (w/v), about 1.5% (w/v), about 2% (w/v), or about 2.5% (w/v). Sodium chloride, when present, may be about 150 mM. In certain compositions, the sodium chloride may be present in higher concentrations, for example from about 200 mM to about 500 mM. In embodiments, the sodium chloride is present in a high concentration, including but not limited to about 200 mM, about 250 mM, about 300 mM, about 350 mM, about 400 mM, about 450 mM, or about 500 mM.

In embodiments, the nanoparticles described herein have improved stability at certain pH levels. In embodiments, the nanoparticles are stable at slightly acidic pH levels. For example, the nanoparticles that are stable at a slightly acidic pH, for example from pH 5.8 to pH 7.0. In embodiments, the nanoparticles and compositions containing nanoparticles may be stable at pHs ranging from about pH 5.8 to about pH 7.0, including about pH 5.9 to about pH 6.8, about pH 6.0 to about pH 6.5, about pH 6.1 to about pH 6.4, about pH 6.1 to about pH 6.3, or about pH 6.2. In embodiments, the nanoparticles and compositions described herein are stabile at neutral pHs, including from about pH 7.0 to about pH 7.4. In embodiments, the nanoparticles and compositions described herein are stable at slightly alkaline pHs, for example from about pH 7.0 to about pH 8.5, from about pH 7.0 to about pH 8.0, or from about pH 7.0 to about pH 7.5, including all values and ranges in between.

### Adjuvants

In embodiments, the compositions disclosed herein may be combined with one or more adjuvants to enhance an immune response. In embodiments, the compositions are prepared without adjuvants, and are thus available to be administered as adjuvant-free compositions. Advantageously, adjuvant-free compositions disclosed herein may provide protective immune responses when administered as a single dose. Alum-free compositions that induce robust immune responses are especially useful in adults about 60 and older.

### Aluminum-Based Adjuvants

In embodiments, the adjuvant may be alum (e.g. AlPO4 or Al(OH)₃). In embodiments, the nanoparticle is substantially bound to the alum. In embodiments, the nanoparticle may be at least 80% bound, at least 85% bound, at least 90% bound or at least 95% bound to the alum. In embodiments, the nanoparticle is 92% to 97% bound to the alum in a composition. The amount of alum is present per dose is typically in a range between about 400 μg to about 1250 μg. For example, the alum may be present in a per dose amount of about 300 μg to about 900 μg, about 400 μg to about 800 μg, about 500 μg to about 700 μg, about 400 μg to about 600 μg, or about 400 μg to about 500 μg. Typically, the alum is present at about 400 μg for a dose of 120 μg of the protein nanoparticle.

### Saponin Adjuvants

Adjuvants containing saponin may also be combined with the immunogens disclosed herein. Saponins are glycosides derived from the bark of the Quillaja saponaria Molina tree. Typically, saponin is prepared using a multi-step purification process resulting in multiple fractions. As used, herein, the term “a saponin fraction from Quillaja saponaria Molina” is used generically to describe a semi-purified or defined saponin fraction of Quillaja saponaria or a substantially pure fraction thereof.

### Saponin Fractions

Several approaches for producing saponin fractions are suitable. Fractions A, B, and C are described in U.S. Pat. No. 6,352,697 and may be prepared as follows. A lipophilic fraction from Quil A, a crude aqueous Quillaja saponaria Molina extract, is separated by chromatography and eluted with 70% acetonitrile in water to recover the lipophilic fraction. This lipophilic fraction is then separated by semi-preparative HPLC with elution using a gradient of from 25% to 60% acetonitrile in acidic water. The fraction referred to herein as “Fraction A” or “QH-A” is, or corresponds to, the fraction, which is eluted at approximately 39% acetonitrile. The fraction referred to herein as “Fraction B” or “QH-B” is, or corresponds to, the fraction, which is eluted at approximately 47% acetonitrile. The fraction referred to herein as “Fraction C” or “QH-C” is, or corresponds to, the fraction, which is eluted at approximately 49% acetonitrile. Additional information regarding purification of Fractions is found in U.S. Pat. No. 5,057,540. When prepared as described herein, Fractions A, B and C of Quillaja saponaria Molina each represent groups or families of chemically closely related molecules with definable properties. The chromatographic conditions under which they are obtained are such that the batch-to-batch reproducibility in terms of elution profile and biological activity is highly consistent.

Other saponin fractions have been described. Fractions B3, B4 and B4b are described in EP 0436620. Fractions QA1-QA22 are described EP03632279 B2, Q-VAC (Nor-Feed, AS Denmark), Quillaja saponaria Molina Spikoside (Isconova AB, Ultunaallén 2B, 756 51 Uppsala, Sweden). Fractions QA-1, QA-2, QA-3, QA-4, QA-5, QA-6, QA-7, QA-8, QA-9, QA-10, QA-11, QA-12, QA-13, QA-14, QA-15, QA-16, QA-17, QA-18, QA-19, QA-20, QA-21, and QA-22 of EP 0 3632 279 B2, especially QA-7, QA-17, QA-18, and QA-21 may be used. They are obtained as described in EP 0 3632 279 B2, especially at page 6 and in Example 1 on page 8 and 9.

The saponin fractions described herein and used for forming adjuvants are often substantially pure fractions; that is, the fractions are substantially free of the presence of contamination from other materials. In particular aspects, a substantially pure saponin fraction may contain up to 40% by weight, up to 30% by weight, up to 25% by weight, up to 20% by weight, up to 15% by weight, up to 10% by weight, up to 7% by weight, up to 5% by weight, up to 2% by weight, up to 1% by weight, up to 0.5% by weight, or up to 0.1% by weight of other compounds such as other saponins or other adjuvant materials.

### ISCOM Structures

Saponin fractions may be administered in the form of a cage-like particle referred to as an ISCOM (Immune Stimulating COMplex). ISCOMs may be prepared as described in EP0109942B1, EP0242380B 1 and EP0180546 B1. In embodiments, a transport and/or a passenger antigen may be used, as described in EP 9600647-3 (PCT/SE97/00289).

### Matrix Adjuvants

In embodiments, the ISCOM is an ISCOM matrix complex. An ISCOM matrix complex comprises at least one saponin fraction and a lipid. The lipid is at least a sterol, such as cholesterol. In embodiments, the ISCOM matrix complex also contains a phospholipid. The ISCOM matrix complexes may also contain one or more other immunomodulatory (adjuvant-active) substances, not necessarily a glycoside, and may be produced as described in EP0436620B1, which is incorporated by reference in its entirety herein.

In other aspects, the ISCOM is an ISCOM complex. An ISCOM complex contains at least one saponin, at least one lipid, and at least one kind of antigen or epitope. The ISCOM complex contains antigen associated by detergent treatment such that that a portion of the antigen integrates into the particle. In contrast, ISCOM matrix is formulated as an admixture with antigen and the association between ISCOM matrix particles and antigen is mediated by electrostatic and/or hydrophobic interactions.

In embodiments, the saponin fraction is integrated into an ISCOM matrix complex or an ISCOM complex, or at least one additional adjuvant, which also is integrated into the ISCOM or ISCOM matrix complex or mixed therewith, is selected from fraction A, fraction B, or fraction C of Quillaja saponaria, a semipurified preparation of Quillaja saponaria, a purified preparation of Quillaja saponaria, or any purified sub-fraction e.g., QA 1-21.

In particular aspects, each ISCOM particle may contain at least two saponin fractions. Any combinations of weight % of different saponin fractions may be used. Any combination of weight % of any two fractions may be used. For example, the particle may contain any weight % of fraction A and any weight % of another saponin fraction, such as a crude saponin fraction or fraction C, respectively. Accordingly, in particular aspects, each ISCOM matrix particle or each ISCOM complex particle may contain from 0.1 to 99.9 by weight, 5 to 95% by weight, 10 to 90% by weight 15 to 85% by weight, 20 to 80% by weight, 25 to 75% by weight, 30 to 70% by weight, 35 to 65% by weight, 40 to 60% by weight, 45 to 55% by weight, 40 to 60% by weight, or 50% by weight of one saponin fraction, e.g. fraction A and the rest up to 100% in each case of another saponin e.g. any crude fraction or any other faction e.g. fraction C. The weight is calculated as the total weight of the saponin fractions. Examples of ISCOM matrix complex and ISCOM complex adjuvants are disclosed in U.S Published Application No. 2013/0129770, which is incorporated by reference in its entirety herein.

In embodiments, the ISCOM matrix or ISCOM complex comprises from 5-99% by weight of one fraction, e.g. fraction A and the rest up to 100% of weight of another fraction e.g. a crude saponin fraction or fraction C. The weight is calculated as the total weight of the saponin fractions.

In embodiment, the ISCOM matrix or ISCOM complex comprises from 40% to 99% by weight of one fraction, e.g. fraction A and from 1% to 60% by weight of another fraction, e.g. a crude saponin fraction or fraction C. The weight is calculated as the total weight of the saponin fractions.

In embodiments, the ISCOM matrix or ISCOM complex comprises from 70% to 95% by weight of one fraction e.g., fraction A, and from 30% to 5% by weight of another fraction, e.g., a crude saponin fraction, or fraction C. The weight is calculated as the total weight of the saponin fractions. In other embodiments, the saponin fraction from Quillaja saponaria Molina is selected from any one of QA 1-21.

In addition to particles containing mixtures of saponin fractions, ISCOM matrix particles and ISCOM complex particles may each be formed using only one saponin fraction. Compositions disclosed herein may contain multiple particles wherein each particle contains only one saponin fraction. That is, certain compositions may contain one or more different types of ISCOM-matrix complexes particles and/or one or more different types of ISCOM complexes particles, where each individual particle contains one saponin fraction from Quillaja saponaria Molina, wherein the saponin fraction in one complex is different from the saponin fraction in the other complex particles.

In embodiments, one type of saponin fraction or a crude saponin fraction may be integrated into one ISCOM matrix complex or particle and another type of substantially pure saponin fraction, or a crude saponin fraction, may be integrated into another ISCOM matrix complex or particle. A composition or vaccine may comprise at least two types of complexes or particles each type having one type of saponins integrated into physically different particles.

In the compositions, mixtures of ISCOM matrix complex particles and/or ISCOM complex particles may be used in which one saponin fraction Quillaja saponaria Molina and another saponin fraction Quillaja saponaria Molina are separately incorporated into different ISCOM matrix complex particles and/or ISCOM complex particles.

The ISCOM matrix or ISCOM complex particles, which each have one saponin fraction, may be present in composition at any combination of weight %. In particular aspects, a composition may contain 0.1% to 99.9% by weight, 5% to 95% by weight, 10% to 90% by weight, 15% to 85% by weight, 20% to 80% by weight, 25% to 75% by weight, 30% to 70% by weight, 35% to 65% by weight, 40% to 60% by weight, 45% to 55% by weight, 40 to 60% by weight, or 50% by weight, of an ISCOM matrix or complex containing a first saponin fraction with the remaining portion made up by an ISCOM matrix or complex containing a different saponin fraction. In aspects, the remaining portion is one or more ISCOM matrix or complexes where each matrix or complex particle contains only one saponin fraction. In other aspects, the ISCOM matrix or complex particles may contain more than one saponin fraction.

In particular compositions, the only saponin fraction in a first ISCOM matrix or ISCOM complex particle is Fraction A and the only saponin fraction in a second ISCOM matrix or ISCOM complex particle is Fraction C.

In embodiments, the Fraction A of Quillaja Saponaria Molina accounts for at least about 80%, 81%, 82%, 83%, 84%, 85%, 86%, 87%, 88%, 89%, 90%, 91%, 92%, 93%, 94%, 95%, 96%, 97%, 98%, or 99% by weight, and fraction C of Quillaja Saponaria Molina accounts for the remainder, respectively, of the sum of the weights of fraction A of Quillaja Saponaria Molina and fraction C of Quillaja Saponaria Molina in the adjuvant.

Preferred compositions comprise a first ISCOM matrix containing Fraction A and a second ISCOM matrix containing Fraction C, wherein the Fraction A ISCOM matrix constitutes about 70% per weight of the total saponin adjuvant, and the Fraction C ISCOM matrix constitutes about 30% per weight of the total saponin adjuvant. In another preferred composition, the Fraction A ISCOM matrix constitutes about 85% per weight of the total saponin adjuvant, and the Fraction C ISCOM matrix constitutes about 15% per weight of the total saponin adjuvant. In another preferred composition, the Fraction A ISCOM matrix constitutes about 92% per weight of the total saponin adjuvant, and the Fraction C ISCOM matrix constitutes about 8% per weight of the total saponin adjuvant. Thus, in certain compositions, the Fraction A ISCOM matrix is present in a range of about 70% to about 85%, and Fraction C ISCOM matrix is present in a range of about 15% to about 30%, of the total weight amount of saponin adjuvant in the composition. In certain compositions, the Fraction A ISCOM matrix is present in a range of about 70% to about 92%, and Fraction C ISCOM matrix is present in a range of about 8% to about 30%, of the total weight amount of saponin adjuvant in the composition. In embodiments, the Fraction A ISCOM matrix accounts for 50-96% by weight and Fraction C ISCOM matrix accounts for the remainder, respectively, of the sums of the weights of Fraction A ISCOM matrix and Fraction C ISCOM in the adjuvant. In a particularly preferred composition, referred to herein as MATRIX-MTM, the Fraction A ISCOM matrix is present at about 85% and Fraction C ISCOM matrix is present at about 15% of the total weight amount of saponin adjuvant in the composition. MATRIX-MTM may be referred to interchangeably as Matrix-M1.

Exemplary QS-7 and QS-21 fractions, their production and their use is described in U.S. Pat. Nos. 5,057,540; 6,231,859; 6,352,697; 6,524,584; 6,846,489; 7,776,343, and 8,173,141, which are incorporated by reference herein.

In embodiments, other adjuvants may be used in addition or as an alternative. The inclusion of any adjuvant described in Vogel et al., “A Compendium of Vaccine Adjuvants and Excipients (2nd Edition),” herein incorporated by reference in its entirety for all purposes, is envisioned within the scope of this disclosure. Other adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalenc/TWEEN® polysorbate 80 emulsion. In embodiments, the adjuvant may be a paucilamellar lipid vesicle; for example, NOVASOMES®. NOVASOMES® are paucilamellar nonphospholipid vesicles ranging from about 100 nm to about 500 nm. They comprise BRIJ® alcohol ethoxylate 72, cholesterol, oleic acid and squalene. NOVASOMES® have been shown to be an effective adjuvant (see, U.S. Pat. Nos. 5,629,021, 6,387,373, and 4,911,928.

### Administration and Dosage

In embodiments, the disclosure provides a method for eliciting an immune response against one or more coronaviruses. In embodiments, the response is against one or more of the SARS-CoV-2 virus, MERS, and SARS. In embodiments, the response is against a heterogeneous SARS-CoV-2 strain. In embodiments, the heterogeneous SARS-COV-2 strain has a World Health Organization Label of alpha, beta, gamma, delta, epsilon, eta, iota, kappa, zeta, mu, or omicron. In embodiments, the heterogeneous SARS-COV-2 strain has a PANGO lineage selected from the group consisting of B.1.1.529; BA.1, BA.1.1, BA.2, BA.3, BA.4, BA.5, B.1.1.7, B.1.351, P.1, B.1.617.2, AY, B.1.427, B.1.429, B.1.525, B.1.526, B.1.617.1, B.1.617.3, P.2, B.1.621, or B.1.621.1. The method involves administering an immunologically effective amount of a composition containing a nanoparticle or containing a recombinant CoV Spike(S) polypeptide to a subject. Advantageously, the proteins disclosed herein induce one or more of particularly useful anti-coronavirus responses.

In embodiments, the nanoparticles or CoV S polypeptides are administered with an adjuvant. In aspects, the nanoparticles or CoV S polypeptides are administered without an adjuvant. In aspects, the adjuvant may be bound to the nanoparticle, such as by a non-covalent interaction. In other aspects, the adjuvant is co-administered with the nanoparticle but the adjuvant and nanoparticle do not interact substantially.

In embodiments, the nanoparticles or CoV S polypeptides may be used for the prevention and/or treatment of one or more of a SARS-COV-2 infection, a heterogeneous SARS-COV-2 strain infection, a SARS infection, or a MERS infection. Thus, the disclosure provides a method for eliciting an immune response against one or more of the SARS-COV-2 virus, heterogeneous SARS-COV-2 virus, MERS, and SARS. The method involves administering an immunologically effective amount of a composition containing a nanoparticle or a CoV S polypeptide to a subject. Advantageously, the proteins disclosed herein induce particularly useful anti-coronavirus responses.

In embodiments, the nanoparticles or CoV S polypeptides described herein have an efficacy against a SARS-COV-2 virus or a heterogeneous SARS-COV-2 strain that is between about 50% and about 99%, between about 80% and about 99%, between about 75% and about 99%, between about 80% and about 95%, between about 90% and about 98%, between about 75% and about 95%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

Compositions disclosed herein may be administered via a systemic route or a mucosal route or a transdermal route or directly into a specific tissue. As used herein, the term “systemic administration” includes parenteral routes of administration. In particular, parenteral administration includes subcutaneous, intraperitoneal, intravenous, intraarterial, intramuscular, or intrasternal injection, intravenous, or kidney dialytic infusion techniques. Typically, the systemic, parenteral administration is intramuscular injection. As used herein, the term “mucosal administration” includes oral, intranasal, intravaginal, intra-rectal, intra-tracheal, intestinal and ophthalmic administration. Preferably, administration is intramuscular.

Compositions may be administered on a single dose schedule or a multiple dose schedule. Multiple doses may be used in a primary immunization schedule or in a booster immunization schedule. In embodiments, about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 doses are administered. In a multiple dose schedule the various doses may be given by the same or different routes e.g., a parenteral prime and mucosal boost, a mucosal prime and parenteral boost, etc. In aspects, a boost dose is administered about 2 weeks, about 3 weeks, about 4 weeks, about 5 weeks, about 6 weeks, about 2 months, about 3 months, about 4 months, about 5 months, about 6 months, about 7 months, about 8 months, about 9 months, about 10 months, about 11 months, about 12 months (1 year), about 2 years, about 3 years, about 4 years, about 5 years, about 6 years, about 7 years, about 8 years, about 9 years, or about 10 years after the first dose. In embodiments, a boost dose is administered every year after administration of the initial dose. In embodiments, the follow-on boost dose is administered 3 weeks or 4 weeks after administration of the prior dose. In embodiments, the first dose is administered at day 0, and the boost dose is administered at day 21. In embodiments, the first dose is administered at day 0, and the boost dose is administered at day 28. In embodiments, the first dose is administered at day 0, a boost dose is administered at day 21, and a second boost dose is administered about six months after administration of the first dose or second dose. In embodiments, the first dose is administered at day 0, and the boost dose is administered at day 28, and a second boost dose is administered about six months after administration of the first dose. In embodiments, the first dose is administered at day 0, a boost dose is administered at day 21, and a second boost dose is administered about six months after administration of the second dose. In embodiments, the first dose is administered at day 0, and the boost dose is administered at day 28, and a second boost dose is administered about six months after administration of the second dose.

In embodiments, the first dose is administered at day 0, a boost dose is administered at day 21, and a second boost dose is administered about 1 year after administration of the first dose or the first boost dose. In embodiments, the first dose is administered at day 0, a first boost dose is administered at day 28, and a second boost dose is administered about 1 year after administration of the first dose. In embodiments, the first dose is administered at day 0, a boost dose is administered at day 21, and a second boost dose is administered about 1 year after administration of the second dose. In embodiments, the first dose is administered at day 0, a first boost dose is administered at day 28, and a second boost dose is administered about 1 year after administration of the second dose. In embodiments, the second boost dose is administered from 6 months to 24 months or from 12 to 24 months after the first boost dose.

In embodiments, the boost dose comprises the same immunological composition as the initial dose. In embodiments, the boost dose comprises a different immunological composition than the initial dose. In embodiments, the different immunological composition is a SARS-COV-2 Spike glycoprotein, an mRNA encoding a SARS-Cov-2 Spike glycoprotein, a plasmid DNA encoding a SARS-Cov-2 Spike glycoprotein, an viral vector encoding a SARS-Cov-2 Spike glycoprotein, or an inactivated SARS-COV-2 virus. In embodiments, the boost dose comprises the initial composition. In embodiments, the initial dose comprises a SARS-COV-2 S glycoprotein (e.g., a SARS COV-2 S glycoprotein having the amino acid sequence of SEQ ID NO: 87), and the boost dose comprises the same SARS-COV-2 S glycoprotein (e.g., a SARS COV-2 S glycoprotein having the amino acid sequence of SEQ ID NO: 87). In embodiments, the initial dose comprises a SARS-COV-2 S glycoprotein (e.g., a SARS COV-2 S glycoprotein having the amino acid sequence of SEQ ID NO: 87), and the boost dose comprises a different SARS-COV-2 S glycoprotein (e.g., a SARS COV-2 S glycoprotein having the amino acid sequence of SEQ ID NO: 132).

In embodiments, the initial dose comprises a combination of SARS-COV-2 S glycoproteins (e.g., a SARS COV-2 S glycoprotein having the amino acid sequence of SEQ ID NO: 87 and a SARS COV-2 S glycoprotein having the amino acid sequence of SEQ ID NO: 132). In embodiments, the boost dose comprises a combination of SARS-COV-2 S glycoproteins (e.g., a SARS COV-2 S glycoprotein having the amino acid sequence of SEQ ID NO: 87 and a SARS CoV-2 S glycoprotein having the amino acid sequence of SEQ ID NO: 132). In embodiments, the initial dose comprises a SARS-COV-2 S glycoprotein, a plasmid DNA encoding a SARS-Cov-2 S glycoprotein, an viral vector encoding a SARS-COV-2 Spike glycoprotein, or an inactivated SARS-COV-2 virus. In embodiments, the initial dose comprises a SARS-COV-2 Spike glycoprotein, a plasmid DNA encoding a SARS-COV-2 Spike glycoprotein, an viral vector encoding a SARS-Cov-2 Spike glycoprotein, or an inactivated SARS-COV-2 virus, and the boost dose comprises one or more SARS-COV-2 S glycoproteins.

In embodiments, the dose, as measured in ug, may be the total weight of the dose including the solute, or the weight of the CoV S polypeptide nanoparticles, or the weight of the CoV S polypeptide. Dose is measured using protein concentration assay either A280 or ELISA.

The dose of antigen, including for pediatric administration, may be in the range of about 5 μg to about 25 μg, about 1 μg to about 300 μg, about 90 μg to about 270 μg, about 100 μg to about 160 μg, about 110 μg to about 150 μg, about 120 μg to about 140 μg, or about 140 μg to about 160 μg. In embodiments, the dose is about 120 μg, administered with alum. In aspects, a pediatric dose may be in the range of about 1 μg to about 90 μg. In embodiments, the dose of CoV Spike(S) polypeptide is about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21, about 22, about 23, about 24, about 25 μg, about 26 μg, about 27 μg, about 28 μg, about 29 μg, about 30 μg, about 40 μg, about 50, about 60, about 70, about 80, about 90 about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, or about 300 μg, including all values and ranges in between. In embodiments, the dose of CoV S polypeptide is 5 μg. In embodiments, the dose of CoV S polypeptide is 25 μg. In embodiments, the dose of a CoV S polypeptide is the same for the initial dose and for boost doses. In embodiments, the dose of a CoV S polypeptide is the different for the initial dose and for boost doses.

In embodiments, the amount of first CoV S glycoprotein in the composition ranges from about 1 μg to about 100 μg. In embodiments, the amount of second, third, fourth, and fifth CoV S glycoproteins is less than the amount of first CoV S glycoprotein in the composition. In embodiments, the immunogenic composition comprises from about 1 ng to about 5 μg of a second CoV S glycoprotein in the immunogenic composition. In embodiments, the immunogenic composition comprises from about 1 ng to about 5 μg of a third CoV S glycoprotein in the immunogenic composition. In embodiments, the immunogenic composition comprises from about 1 ng to about 5 μg of a fourth CoV S glycoprotein in the immunogenic composition. In embodiments, the immunogenic composition comprises from about 1 ng to about 5 μg of a fifth CoV S glycoprotein in the immunogenic composition. In embodiments, the total amount of CoV S glycoprotein in the immunogenic composition is greater than I ug and less than about 5 μg, less than about 10 μg, less than about 15 μg, less than about 20 μg, or less than about 25 μg.

Certain populations may be administered with or without adjuvants. In certain aspects, compositions may be free of added adjuvant. In such circumstances, the dose may be increased by about 10%.

In embodiments, the immunogenic compositions described herein are provided in pre-filled syringes. When the immunogenic composition is prepared in a pre-filled syringe, the CoV S polypeptides and adjuvant are combined in advance of administration.

In embodiments, the dose of the adjuvant administered with a CoV S polypeptide, including naturally and non-naturally occurring polypeptides, is from about 1 μg to about 100 μg, for example, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21, about 22, about 23, about 24, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, or about 100 μg of adjuvant. In embodiments, the dose of adjuvant is about 50 μg. In embodiments, the adjuvant is a saponin adjuvant, e.g., MATRIX-M™

In embodiments, the dose is administered in a volume of about 0.1 mL to about 1.5 mL, for example, about 0.1 mL, about 0.2 mL, about 0.25 mL, about 0.3 mL, about 0.4 mL, about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, or about 1.5 mL. In embodiments, the dose is administered in a volume of 0.25 mL. In embodiments, the dose is administered in a volume of 0.5 mL. In embodiments, the dose is administered in a volume of 0.6 mL.

In particular embodiments for a vaccine against MERS, SARS, or the SARS-COV-2 coronavirus, the dose may comprise a CoV S polypeptide concentration of about 1 μg/mL to about 50 μg/mL, 10 μg/mL to about 100 μg/mL, about 10 μg/mL to about 50 μg/mL, about 175 μg/mL to about 325 μg/mL, about 200 μg/mL to about 300 μg/mL, about 220 μg/mL to about 280 μg/mL, or about 240 μg/mL to about 260 μg/mL.

In another embodiment, the disclosure provides a method of formulating a vaccine composition that induces immunity to an infection or at least one disease symptom thereof to a mammal, comprising adding to the composition an effective dose of a nanoparticle or a CoV S polypeptide. The disclosed CoV S polypeptides and nanoparticles are useful for preparing compositions that stimulate an immune response that confers immunity or substantial immunity to infectious agents. Thus, in one embodiment, the disclosure provides a method of inducing immunity to infections or at least one disease symptom thereof in a subject, comprising administering at least one effective dose of a nanoparticle and/or a CoV S polypeptide.

In embodiments, the CoV S polypeptides or nanoparticles comprising the same are administered in combination with an additional immunogenic composition. In embodiments, the additional immunogenic composition induces an immune response against SARS-COV-2. In embodiments, the additional immunogenic composition is administered within about 1 minute, about 5 minutes, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days of the disclosed CoV S polypeptides or nanoparticles comprising the same. In embodiments, the additional composition is administered with a first dose of a composition comprising a CoV S polypeptide or nanoparticle comprising the same. In embodiments, the additional composition is administered with a boost dose of a composition comprising a CoV S polypeptide or nanoparticle comprising the same.

In embodiments, the additional immunogenic composition comprises an mRNA encoding a SARS-Cov-2 Spike glycoprotein, a plasmid DNA encoding a SARS-Cov-2 Spike glycoprotein, an viral vector encoding a SARS-Cov-2 Spike glycoprotein, or an inactivated SARS-COV-2 virus.

In embodiments, the additional immunogenic composition comprises mRNA that encodes for a CoV S polypeptide. In embodiments, the mRNA encodes for a CoV S polypeptide comprising proline substitutions at positions 986 and 987 of SEQ ID NO: 1. In embodiments, the mRNA encodes for a CoV S polypeptide comprising an intact furin cleavage site. In embodiments, the mRNA encodes for a CoV S polypeptide comprising proline substitutions at positions 986 and 987 of SEQ ID NO: 1 and an intact furin cleavage site. In embodiments, the mRNA encodes for a CoV S polypeptide comprising proline substitutions at positions 986 and 987 of SEQ ID NO: 1 and an inactive furin cleavage site. In embodiments, the mRNA encodes for a CoV S polypeptide having an amino acid sequence of SEQ ID NO: 87. In embodiments, the mRNA encoding for a CoV S polypeptide is encapsulated in a lipid nanoparticle. An exemplary immunogenic composition comprising mRNA that encodes for a CoV S polypeptide is described in Jackson et al. N. Eng. J. Med. 2020. An mRNA Vaccine against SARS-COV-2-preliminary report, which is incorporated by reference in its entirety herein. In embodiments, the composition comprising mRNA that encodes for a CoV S polypeptide is administered at a dose of 25 μg, 100 μg, or 250 μg.

In embodiments, the additional immunogenic composition comprises an adenovirus vector encoding for a CoV S polypeptide. In embodiments, the AAV vector encodes for a wild-type CoV S polypeptide. In embodiments, the AAV vector encodes for a CoV S polypeptide comprising proline substitutions at positions 986 and 987 of SEQ ID NO: 1 and an intact furin cleavage site. In embodiments, the AAV vector encodes for a CoV S polypeptide comprising proline substitutions at positions 986 and 987 of SEQ ID NO: 1 and an inactive furin cleavage site. In embodiments, the AAV vector encodes for a CoV S polypeptide having an amino acid sequence of SEQ ID NO: 87. The following publications describe immunogenic compositions comprising an adenovirus vector encoding for a CoV S polypeptide, each of which is incorporated by reference in its entirety herein: van Doremalen N. et al. A single dose of ChAdOx1 MERS provides protective immunity in rhesus macaques. Science Advances, 2020; van Doremalen N. et al. ChAdOx1 nCOV-19 vaccination prevents SARS-COV-2 pneumonia in rhesus macaques. bioRxiv, (2020).

In embodiments, the additional immunogenic composition comprises deoxyribonucleic acid (DNA). In embodiments, the additional immunogenic composition comprises plasmid DNA. In embodiments, the plasmid DNA encodes for a CoV S polypeptide. In embodiments, the DNA encodes for a CoV S polypeptide comprising proline substitutions at positions 986 and 987 of SEQ ID NO: 1 and an intact furin cleavage site. In embodiments, the DNA encodes for a CoV S polypeptide comprising proline substitutions at positions 986 and 987 of SEQ ID NO: 1 and an inactive furin cleavage site. In embodiments, the DNA encodes for a CoV S polypeptide having an amino acid sequence of SEQ ID NO: 87. In embodiments, the DNA encodes for a CoV S polypeptide having an amino acid sequence of SEQ ID NO: 174 or SEQ ID NO: 175.

In embodiments, the additional immunogenic composition comprises an inactivated virus vaccine.

In embodiments, the CoV S polypeptides or nanoparticles comprising CoV S polypeptides are administered to a patient that has or has previously had a confirmed infection caused by SARS-CoV-2 or a heterogeneous SARS-COV-2 strain. The infection with SARS-COV-2 or a heterogeneous SARS-COV-2 strain may be confirmed by a nucleic acid amplification test (e.g., polymerase chain reaction) or serological testing (e.g., testing for antibodies against a SARS-COV-2 viral antigen). In embodiments, the CoV S polypeptides or nanoparticles comprising CoV S polypeptides are administered to a patient at least about 3 days, at least about 1 week, at least about 2 weeks, at least about 3 weeks, at least about 4 weeks after a patient has been diagnosed with COVID-19. In embodiments, the CoV S polypeptides or nanoparticles comprising CoV S polypeptides are administered to a patient between 1 week and 1 year after the patient's diagnosis with COVID-19, for example, about 1 week, about 2 weeks, about 3 weeks, about 4 weeks, about 5 weeks, about 6 weeks, about 1 month, about 2 months, about 3 months, about 4 months, about 5 months, about 6 months, about 7 months, about 8 months, about 9 months, about 10 months, about 11 months, or about 1 year. In embodiments, the CoV S polypeptides or nanoparticles comprising CoV S polypeptides are administered to a patient between I week and 20 years after the patient's diagnosis with COVID-19, for example, about 1 week, about 2 weeks, about 3 weeks, about 4 weeks, about 5 weeks, about 6 weeks, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, or about 20 years.

In embodiments, the CoV S polypeptides or nanoparticles comprising the same are administered after the patient has been administered a first immunogenic composition. Non-limiting examples of first immunogenic compositions include a SARS-COV-2 Spike glycoprotein, an mRNA encoding a SARS-Cov-2 Spike glycoprotein, a plasmid DNA encoding a SARS-Cov-2 Spike glycoprotein, an viral vector encoding a SARS-Cov-2 Spike glycoprotein, or an inactivated SARS-COV-2 virus. In embodiments, the CoV S polypeptides or nanoparticles comprising the same are administered between about 1 week and about 1 year, between about I week and 1 month, between about 3 weeks and 4 weeks, between about 1 week and 5 years, between about 1 year and about 5 years, between about 1 year and about 3 years, between about 3 years and about 5 years, between about 5 years and about 10 years, between about 1 year and about 10 years, or between about 1 year and about 2 years after administration of the first immunogenic composition. In embodiments, the CoV S polypeptides or nanoparticles comprising the same are administered between about 1 week and about 1 year after administration of the first immunogenic composition, for example, about 1 week, about 2 weeks, about 3 weeks, about 4 weeks, about 5 weeks, about 6 weeks, about 7 weeks, about 8 weeks, about 9 weeks, about 10 weeks, about 1 month, about 2 months, about 3 months, about 4 months, about 5 months, about 6 months, about 7 months, about 8 months, about 9 months, about 10 months, about 11 months, or about 1 year after administration of the first immunogenic composition.

In embodiments, the CoV S proteins or nanoparticles comprising CoV S proteins are useful for preparing immunogenic compositions to stimulate an immune response that confers immunity or substantial immunity to one or more of MERS, SARS, SARS-COV-2, and a heterogeneous SARS-COV-2 strain. Both mucosal and cellular immunity may contribute to immunity to infection and disease. Antibodies secreted locally in the upper respiratory tract are a major factor in resistance to natural infection. Secretory immunoglobulin A (sIgA) is involved in protection of the upper respiratory tract and serum IgG in protection of the lower respiratory tract. The immune response induced by an infection protects against reinfection with the same virus or an antigenically similar viral strain. The antibodies produced in a host after immunization with the nanoparticles disclosed herein can also be administered to others, thereby providing passive administration in the subject.

In embodiments, the CoV S proteins or nanoparticles comprising CoV S proteins induce cross-neutralizing antibodies against SARS-COV-2 viruses containing S proteins with one or more modifications selected from: deletion of one or more amino acids selected from the group consisting of amino acid 11-14, 56, 57, 130, 131, 132, 144, 145, 198, 199, 228, 229, 230, 231, 234, 235, 236, 237, 238, 239, 240, 676-685, 676-702, 702-711, 775-793, 806-815 and combinations thereof; (b) mutation of one or more amino acids selected from the group consisting of amino acid 5, 6, 7, 11, 12, 13, 14, 51, 53, 54, 56, 57, 62, 63, 67, 70, 82, 125, 129, 131, 132, 133, 134, 139, 143, 144, 145, 170, 177, 197, 198, 199, 200, 201, 202, 209, 229, 233, 239, 240, 244, 245, 326, 333, 355, 358, 360, 362, 363, 392, 395, 404, 419, 426, 427, 431, 432, 433, 439, 440, 447, 464, 465, 471, 473, 477, 480, 481, 483, 485, 488, 492, 534, 557, 591, 600, 601, 626, 642, 645, 664, 666, 668, 688, 691, 703, 751, 783, 843, 846, 875, 937, 941, 956, 968, 969, 1014, 1058, 1105, 1163, 1186 and combinations thereof; or (c) insertion of a tripeptide having the amino acid sequence of EPE between amino acids 214 and 215; wherein the amino acids of the CoV S glycoprotein are numbered with respect to a polypeptide having the sequence of SEQ ID NO: 2.

In embodiments, the CoV S proteins or nanoparticles comprising CoV S proteins induce cross-neutralizing antibodies against SARS-COV-2 viruses containing S proteins with one or more modifications selected from: deletions of amino acid 56, deletion of amino acid 57, deletion of amino acid 131, N488Y, A557D, D601G, P668H, T7031, S969A, D1105H, N426K, and Y440F, wherein the amino acids are numbered with respect to a CoV S polypeptide having an amino acid sequence of SEQ ID NO: 2.

In embodiments, the CoV S protein or nanoparticle comprising a CoV S protein induces cross-neutralizing antibodies against SARS-COV-2 viruses containing S proteins with one or more modifications selected from: deletions of amino acid 56, deletion of amino acid 57, deletion of amino acid 131, N488Y, A557D, D601G, P668H, T7031, S969A, and D1105H, wherein the amino acids are numbered with respect to a CoV S polypeptide having an amino acid sequence of SEQ ID NO: 2

In embodiments, the CoV S protein or nanoparticle comprising a CoV S protein induces cross-neutralizing antibodies against SARS-COV-2 viruses containing S proteins with one or more modifications selected from: D67A, D202G, L229H, K404N, E471K, N488Y, D601G, and A688V, wherein the amino acids are numbered with respect to a CoV S polypeptide having an amino acid sequence of SEQ ID NO: 2.

In embodiments, the CoV S protein or nanoparticle comprising a CoV S protein induces cross-neutralizing antibodies against SARS-COV-2 viruses containing S proteins with one or more modifications selected from: deletion of amino acids 229-231, D67A, D202G, K404N, E471K, N488Y, D601G, and A688V, wherein the amino acids are numbered with respect to a CoV S polypeptide having an amino acid sequence of SEQ ID NO: 2.

In embodiments, the CoV S protein or nanoparticle comprising a CoV S protein induces cross-neutralizing antibodies against SARS-COV-2 viruses containing S proteins with one or more modifications selected from: deletion of amino acids 229-231, L5F, D67A, D202G, K404N, E471K, N488Y, D601G, and A688V wherein the amino acids are numbered with respect to a CoV S polypeptide having an amino acid sequence of SEQ ID NO: 2.

In embodiments, the CoV S protein or nanoparticle comprising a CoV S protein induces cross-neutralizing antibodies against SARS-COV-2 viruses containing S proteins with one or more modifications selected from: L5F, T7N, P13S, D125Y, R177S, K404T, E471K, N488Y, D601G, H642Y, T1014I, and V1163F, wherein the amino acids are numbered with respect to a CoV S polypeptide having an amino acid sequence of SEQ ID NO: 2.

In embodiments, the CoV S protein or nanoparticle comprising a CoV S protein induces cross-neutralizing antibodies against SARS-COV-2 viruses with an S protein comprising one or more modifications selected from: W139C and L439R, wherein the amino acids are numbered with respect to a CoV S polypeptide having an amino acid sequence of SEQ ID NO: 2. In embodiments, the CoV S protein comprising W139C and L439R modifications is expressed with a signal peptide having an amino acid sequence of SEQ ID NO: 117 or SEQ ID NO: 5. In embodiments, the CoV S protein or nanoparticle comprising a CoV S protein induces cross-neutralizing antibodies against SARS-COV-2 viruses with one or more modifications selected from: D601G, W139C, and L439R, wherein the amino acids are numbered with respect to a CoV S polypeptide having an amino acid sequence of SEQ ID NO: 2. In embodiments, the CoV S protein or nanoparticle comprising D601G, W139C, and L439R modifications is expressed with a signal peptide having an amino acid sequence of SEQ ID NO: 117 or SEQ ID NO: 5.

In embodiments, the CoV S protein or nanoparticle comprising a CoV S protein induces cross-neutralizing antibodies against SARS-COV-2 viruses with one or more modifications selected from: D601G, L5F, D67A, D202G, deletions of amino acids 229-231, R233I, K404N, E471K, N488Y, and A688V, wherein the amino acids are numbered with respect to a CoV S polypeptide having an amino acid sequence of SEQ ID NO: 2. In embodiments, the CoV S protein or nanoparticle comprising a CoV S protein induces cross-neutralizing antibodies against SARS-CoV-2 viruses with one or more modifications selected from: L5F, D67A, D202G, deletions of amino acids 229-231, R233I, K404N, E471K, N488Y, and A688V, wherein the amino acids are numbered with respect to a CoV S polypeptide having an amino acid sequence of SEQ ID NO: 2.

In embodiments, the present disclosure provides a method of producing one or more of high affinity anti-MERS-COV, anti-SARS-COV, and anti-SARS-COV-2 virus antibodies. The high affinity antibodies produced by immunization with the nanoparticles disclosed herein are produced by administering an immunogenic composition comprising an S CoV polypeptide or a nanoparticle comprising an S CoV polypeptide to an animal, collecting the serum and/or plasma from the animal, and purifying the antibody from the serum/and or plasma. In one embodiment, the animal is a human. In embodiments, the animal is a chicken, mouse, guinea pig, rat, rabbit, goat, human, horse, sheep, or cow. In one embodiment, the animal is bovine or equine. In another embodiment, the bovine or equine animal is transgenic. In yet a further embodiment, the transgenic bovine or equine animal produces human antibodies. In embodiments, the animal produces monoclonal antibodies. In embodiments, the animal produces polyclonal antibodies. In one embodiment, the method further comprises administration of an adjuvant or immune stimulating compound. In a further embodiment, the purified high affinity antibody is administered to a human subject. In one embodiment, the human subject is at risk for infection with one or more of MERS, SARS, and SARS-COV-2.

In embodiments, the CoV S proteins or nanoparticles are co-administered with an influenza glycoprotein or nanoparticle comprising an influenza glycoprotein or a respiratory syncytial virus (RSV) fusion (F) glycoprotein. In embodiments, the CoV S proteins or nanoparticles are co-formulated with RSV F glycoproteins, influenza glycoproteins, or a combination thereof. Suitable glycoproteins and nanoparticles are described in US Publication No. 2018/0133308 and US Publication No. 2019/0314487, each of which is incorporated by reference herein in its entirety. In embodiments, the CoV S protein or nanoparticle is coadministered with: (a) a detergent-core nanoparticle, wherein the detergent-core nanoparticle comprises a recombinant influenza hemagglutinin (HA) glycoprotein from a Type B influenza strain; and (b) a Hemagglutinin Saponin Matrix Nanoparticle (HaSMaN), wherein the HaSMaN comprises a recombinant influenza HA glycoprotein from a Type A influenza strain and ISCOM matrix adjuvant. In embodiments, the CoV S protein or nanoparticle is coadministered with a nanoparticle comprising a non-ionic detergent core and an influenza HA glycoprotein, wherein the influenza HA glycoprotein contains a head region that projects outward from the non-ionic detergent core and a transmembrane domain that is associated with the non-ionic detergent core, wherein the influenza HA glycoprotein is a HA0 glycoprotein, wherein the amino acid sequence of the influenza HA glycoprotein has 100% identity to the amino acid sequence of the native influenza HA protein. In embodiments, the influenza glycoprotein or nanoparticle is coformulated with the CoV S protein or nanoparticle.

In some embodiments, the disclosure provides co-formulation (i.e., pre-filled syringes or pre-mix) strategies for immunogenic compositions comprising a CoV S glycoprotein and an adjuvant (e.g., a saponin adjuvant). Typical vaccine administration strategies currently being utilized are bedside mix formulations. That is, vaccine compositions and adjuvants are stored separately and are mixed prior to administration. Pre-mix, co-formulation, or pre-filled syringe strategies for vaccine are less common due to the concerns of the stability of the antigens (e.g., a CoV S glycoprotein) and their subsequent immunogenic capabilities. The present disclosure provides immunogenic compositions that can be pre-mixed and stored in advance. The disclosed vaccination strategies and formulations may improve the efficiency of vaccination and may reduce the risks of bedside mixing errors, while maintaining the overall safety and immunogenicity.

A variety of containers may be used to store and transport the pre-mix formulations, including syringes for single administrations and plastic ampules. In some instances, plastic ampules can be manufactured using the blow-fill-seal manufacturing technique or method. In general, the blow-fill-seal (BFS) manufacturing method includes extruding a plastic material (e.g., resin) to form a parison, which is then placed into a mold and cut to size. A filling needle or mandrel is then used to inflate the plastic, which in turn, results in a hollow ampule that substantially conforms to the shape of the mold. Once inflated, a desired volume of liquid can be injected into the ampule, the filling needle or mandrel can be removed, and the ampule can be sealed. Accordingly, BFS can be an automated process that can be performed in a sterile environment without direct human intervention.

In some instances, the ability to aseptically manufacture sterile ampules containing a desired liquid can make BFS manufactured ampules particularly well suited for the pharmaceutical industry. BFS technology, however, has not been compatible with all pharmaceutical liquids, products, etc. For example, some known BFS manufacturing methods include delivering the liquid or product into the ampule while the plastic is still relatively hot, which can result in adverse effects to temperature sensitive liquids and/or products such as vaccines, biologics, etc. Advances in cool BFS technology, however, have increased the variety of suitable products, liquids, etc. allowing some vaccines, biologics, and/or other temperature sensitive pharmaceuticals to be contained in BFS ampules.

In some instances, a BFS ampule can have a size, shape, and/or configuration that is at least partially based on a desired use and/or a desired pharmaceutical liquid or dosage that the ampule is configured to contain. For example, some known BFS ampules can include a pierce through top, a twist-off top, a top including a male or female luer, and/or the like. Some known BFS ampules can have a size and/or shape based on volume of the liquid or dosage configured to be disposed therein. In addition, some known BFS ampules can be manufactured in a strip of multiple, temporarily connected ampules, which can increase manufacturing, packaging, and/or storing efficiencies and/or the like.

In embodiments, the immunogenic compositions described herein are provided in pre-filled syringes. When the immunogenic composition is prepared in a pre-filled syringe, an antigen and adjuvant is combined in advance of administration. In embodiments, the pre-filled syringe contains a CoV S glycoprotein and an adjuvant (e.g., a saponin adjuvant). In embodiments, the pre-filled syringe contains a CoV S glycoprotein and a saponin adjuvant, wherein the adjuvant comprises at least two iscom particles, wherein the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina; wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the adjuvant. In embodiments, the pre-filled syringe contains a CoV S glycoprotein and a saponin adjuvant, wherein the adjuvant comprises at least two iscom particles, wherein the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina; wherein fraction A of Quillaja Saponaria Molina and fraction C of Quillaja Saponaria Molina account for about 92% by weight and about 8% by weight, respectively, of the sum of weights of fraction A of Quillaja Saponaria Molina and fraction C of Quillaja Saponaria Molina in the adjuvant. In embodiments, the pre-filled syringe contains a CoV S glycoprotein and a saponin adjuvant, wherein the adjuvant comprises at least two iscom particles, wherein the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina; wherein fraction A of Quillaja Saponaria Molina accounts for at least about 75% by weight and fraction C of Quillaja Saponaria Molina accounts for the remainder of the sum of weights of fraction A of Quillaja Saponaria Molina and fraction C of Quillaja Saponaria Molina in the adjuvant.

### EXAMPLES

### Example 1

### Expression and Purification of Pre-Fusion Coronavirus Spike(S) Polypeptide Nanoparticles

The native coronavirus (CoV) Spike(S) polypeptide (SEQ ID NO: 1 and SEQ ID NO:2) and CoV Spike polypeptides which have amino acid sequences corresponding to SEQ ID NOS: 3, 4, 38, 41, 44, 48, 51, 54, 58, 61, 63, 65, 67, 73, 75, 78, 79, 82, 83, 85, 87,106,108, 89, 112-115, 132, 133, 114, 138, 141, 144, 147, 151, 153, 156, 158, 174-176, 186, 188, 190, 195, 217-228, 233-236, and 245-292 were expressed in a baculovirus expression system and recombinant plaques expressing the CoV S polypeptides were picked and confirmed. In each case the signal peptide was SEQ ID NO: 5, 154, 193, or 117. Additionally, CoV S polypeptides encoded by a nucleic acid of any one of SEQ ID NOS: 161, 162, 163, 164, 165, 166, 168, 169, 171, 172, 196-199, 201, 202, 204, 206, 208, 210, 212, 214, 216, and 237-292 were produced. Additionally, CoV S polypeptides that comprise the polypeptide sequence of SEQ ID NOS: 87, 159, 167, 160, 170, 174, 175, 186, 188, 190, 195, 217-228, 233-236, 243, 255-264, 273-280, 283, 284, 287, 288, 291, 292, 294, 296, 298, 300, 302, 304, 306, 308, 310, 312, 314, 316, 318, 320, 322, 324, 326, 328, 329, 330, 331, 332, 333, and 334 which is C-terminal to a signal peptide having the amino acid sequence of any one of SEQ ID NOS: 5, 154, 193, or 117 were produced.

Table 2A shows the sequence characteristics of the aforementioned CoV Spike polypeptides. Table 2B shows the sequences of additional CoV Spike polypeptides that are being produced. The CoV S polypeptides of Table 2B have from about 1 to about 50 modifications compared to a CoV S polypeptide of Table 2A. The modifications may be in the RBD, NTD, SD1/2, S1 subunit, or S2 subunit. The modifications may also be proximal to the furin cleavage domain.

TABLE 2A
Selected CoV Spike Polypeptides

| CoV S polypeptide | SEQ ID NO. |
|---|---|
| BV2364 | 48 |
| BV2365 | 4 |
| BV2361/BV2366 | 2 |
| BV2367 | 63 |
| BV2368 | 65 |
| BV2369 | 67 |
| BV2373, formulated into a composition referred | 87 |
| to herein as “NVX-CoV2373” | |
| BV2374 | 85 |
| BV2374 | 58 |
| BV2384 | 110 |
| BV2425 | 114 |
| BV2426 | 115 |
| BV2438 | 132 |
| BV2423 | 133 |
| BV2425 | 114 |
| BV2425-2 | 138 |
| BV2439 | 141 |
| BV2441 | 144 |
| BV2442 | 147 |
| BV2443 | 151 |
| BV2448 | 153 |
| BV1.526NY-1 | 156 |
| BV1.526NY-2 | 158 |
| BV2508 | 174 |
| BV2509 | 175 |
| BV2465 | 176 |
| BV2457 | 181 |
| BV2472 | 182 |
| BV2480 | 183 |
| BV2481 | 184 |
| BV2523 | 195 |
| BV2540 | 192 |
| BV2541 | 190 |
| BV2515 | 188 |
| BV2542 | 186 |
| BV2515-1 | 217 |
| BV2523-1 | 218 |
| BV2540-1 | 222 |
| BV2541-1 | 221 |
| BV2549 | 219 |
| BV2542-2 | 220 |
| BV2562 | 225 |
| BV2559 | 223 |
| BV2577 | 224 |
| BV2578 | 226 |
| BV2589 | 227 |
| BV2590 | 228 |
| BV2592 | 233 |
| XBB.1 | 234 |
| BV2591 | 235 |
| BN.1 | 236 |
| Deltacron | 243 |
| BV2561 | 244 |

TABLE 2B
Selected CoV Spike Polypeptides

| CoV S polypeptide | SEQ ID NO. |
|---|---|
| 1A | 245 |
| 1B | 246 |
| 1C | 247 |
| 1D | 248 |
| 1E | 249 |
| 1A-1 | 250 |
| 1B-1 | 251 |
| 1C-1 | 252 |
| 1D-1 | 253 |
| 1E-1 | 254 |
| 1A-2 | 255 |
| 1B-2 | 256 |
| 1C-2 | 257 |
| 1D-2 | 258 |
| 1E-2 | 259 |
| 1A-3 | 260 |
| 1B-3 | 261 |
| 1C-3 | 262 |
| 1D-3 | 263 |
| 1E-3 | 264 |
| 2A | 265 |
| 2B | 266 |
| 2C | 267 |
| 2D | 268 |
| 2A-1 | 269 |
| 2B-1 | 270 |
| 2C-1 | 271 |
| 2D-1 | 272 |
| 2A-2 | 273 |
| 2B-2 | 274 |
| 2C-2 | 275 |
| 2D-2 | 276 |
| 2A-3 | 277 |
| 2B-3 | 278 |
| 2C-3 | 279 |
| 2D-3 | 280 |
| 3A-1 | 281 |
| 3A-2 | 285 |
| 3A-3 | 289 |
| 3B-1 | 282 |
| 3B-2 | 286 |
| 3B-3 | 290 |
| 3C-1 | 283 |
| 3C-2 | 287 |
| 3C-3 | 291 |
| 3D-1 | 284 |
| 3D-2 | 288 |
| 3D-3 | 292 |

### Protein and Nanoparticle Production

The recombinant baculovirus was amplified by infection of Sf9 or rhabdovirus-free Sf22a insect cells. A culture of insect cells was infected at ˜0.6 MOI (Multiplicity of infection=virus ffu or pfu/cell) with baculovirus. The insect cell culture may be infected with baculoviruses that express different CoV S glycoproteins. For example, the insect cell culture may be infected with baculoviruses expressing five different CoV S glycoproteins. The culture and supernatant is harvested 48-72 hrs post-infection. The crude cell harvest, approximately 30 mL, is clarified by centrifugation for 15 minutes at approximately 800×g. The resulting crude cell harvests containing the coronavirus Spike(S) protein are purified as nanoparticles as described below.

To produce nanoparticles, non-ionic surfactant TERGITOL® nonylphenol ethoxylate NP-9 is used in the membrane protein extraction protocol. Crude extraction is further purified by passing through anion exchange chromatography, lentil lectin affinity/HIC and cation exchange chromatography. The washed cells are lysed by detergent treatment and then subjected to low pH treatment which leads to precipitation of BV and Sf9 host cell DNA and protein. The neutralized low pH treatment lysate is clarified and further purified on anion exchange and affinity chromatography before a second low pH treatment is performed.

Affinity chromatography is used to remove Sf9/BV proteins, DNA and NP-9, as well as to concentrate the coronavirus Spike(S) protein. Briefly, lentil lectin is a metalloprotein containing calcium and manganese, which reversibly binds polysaccharides and glycosylated proteins containing glucose or mannose. The coronavirus Spike(S) protein-containing anion exchange flow through fraction is loaded onto the lentil lectin affinity chromatography resin (Capto Lentil Lectin, GE Healthcare). The glycosylated coronavirus Spike(S) protein is selectively bound to the resin while non-glycosylated proteins and DNA are removed in the column flow through. Weakly bound glycoproteins are removed by buffers containing high salt and low molar concentration of methyl alpha-D-mannopyranoside (MMP).

The column washes are also used to detergent exchange the NP-9 detergent with the surfactant polysorbate 80 (PS80). The coronavirus Spike(S) polypeptides are eluted in nanoparticle structure from the lentil lectin column with a high concentration of MMP. After elution, the coronavirus Spike(S) protein trimers are assembled into nanoparticles composed of coronavirus Spike(S) protein trimers and PS80 contained in a detergent core.

Binding of CoV S Polypeptides to hACE2

The ability of the CoV S polypeptides to bind to hACE2 is evaluated by bio-layer interferometry and ELISA.

### Bio-Layer Interferometry (BLI)

The BLI experiments are performed using an Octet QK384 system (Pall Forté Bio, Fremont, CA). His-tagged human ACE2 (2 μg mL-1) was immobilized on nickel-charged Ni-NTA biosensor tips. After baseline, SARS-COV-2 S protein containing samples were 2-fold serially diluted and were allowed to associate for 600 seconds followed by dissociation for an additional 900 sec. Data is analyzed with Octet software HT 101:1 global curve fit.

The CoV S polypeptides of Table 2A retained the ability to bind to hACE2. Dissociation kinetics showed that the CoV S polypeptides remained tightly bound as evident by minimal or no dissociation over 900 seconds of observation in the absence of fluid phase S protein. The ability of the CoV S polypeptides of Table 2B to bind to hACE2 by BLI is evaluated.

### ELISA

The specificity of the CoV S polypeptides for hACE2 was confirmed by ELISA. Ninety-six well plates were coated with 100 μL SARS-COV-2 spike protein (2 μg/mL) overnight at 4° C. Plates were washed with phosphate buffered saline with 0.05% Tween (PBS-T) buffer and blocked with TBS Startblock blocking buffer (ThermoFisher, Scientific). His-tagged hACE2 and hDPP4 receptors were 3-fold serially diluted (5-0.0001 μg mL-1) and added to coated wells for 2 hours at room temperature. The plates were washed with PBS-T. Optimally diluted horseradish peroxidase (HRP) conjugated anti-histidine was added and color developed by addition of and 3,3′,5,5′-tetramethylbenzidine peroxidase substrate (TMB, T0440-IL, Sigma, St. Louis, MO, USA). Plates were read at an OD of 450 nm with a SpectraMax Plus plate reader (Molecular Devices, Sunnyvale, CA, USA) and data analyzed with SoftMax software. EC50 values were calculated by 4-parameter fitting using GraphPad Prism 7.05 software.

### Example 2

### Formulation of Pentavalent COVID-19 Immunogenic Composition

Pentavalent COVID-19 immunogenic compositions are formulated. The pentavalent COVID-19 immunogenic comprises five CoV S glycoproteins. Each of the first, second, third, fourth, and fifth CoV S glycoproteins may have an inactivated furin cleavage site. The inactivated furin cleavage site may comprise the amino sequence of QQAQ (SEQ ID NO: 7). Each of the first, second, third, fourth, and fifth CoV S glycoproteins may also have modifications at amino acids 986 and 987, wherein each CoV S glycoprotein is numbered with respect to a CoV S glycoprotein of SEQ ID NO: 1. Amino acids 986 and 987 of one or more of the first, second, third, fourth, and fifth CoV S glycoproteins may be proline.

Table 2C1 shows the identity of the five CoV S glycoproteins in each immunogenic composition. The second, third, fourth, and fifth CoV S glycoproteins have one or more modifications compared to the first CoV S glycoprotein. Each of the first, second, third, fourth, and fifth CoV S glycoproteins in the immunogenic compositions have different amino acid sequences. Table 2C2 shows possible first, second, third, fourth, and fifth CoV S glycoproteins in the compositions and shows their modifications relative to a CoV S glycoprotein of SEQ ID NO: 1.

Compared to the first CoV S glycoprotein in the composition, one or more of the second, third, fourth, and fifth CoV S glycoprotein may have modifications in the receptor binding domain, N-terminal domain, S1 subunit, S2 subunit, or SD1/2. The modifications may occur at positions 180, 252, 253, 444, 478, 486, 521, or a combination thereof, wherein the modifications are numbered with respect to a CoV S polypeptide of SEQ ID NO: 1. Position 180 of the CoV S polypeptide may be glutamate or valine, wherein the CoV S polypeptide is numbered within respect to SEQ ID NO: 1. Position 252 of the CoV S polypeptide may be glycine or valine, wherein the CoV S polypeptide is numbered within respect to SEQ ID NO: 1. Position 253 of the CoV S polypeptide may be aspartate or glycine, wherein the CoV S polypeptide is numbered within respect to SEQ ID NO: 1. Position 444 of the CoV S polypeptide may be threonine or lysine, wherein the CoV S polypeptide is numbered within respect to SEQ ID NO: 1. Position 478 of the CoV S polypeptide may be threonine, arginine, or lysine, wherein the CoV S polypeptide is numbered within respect to SEQ ID NO: 1. Position 486 of the CoV S polypeptide may be threonine or serine wherein the CoV S polypeptide is numbered within respect to SEQ ID NO: 1. Position 521 of the CoV S polypeptide may be proline or serine, wherein the CoV S polypeptide is numbered within respect to SEQ ID NO: 1.

TABLE 2C1

| | | Second, Third, Fourth, and |
|---|---|---|
| Composition | First CoV S Glycoprotein | Fifth CoV S Glycoprotein |
| 1 | A CoV S glycoprotein with at least 90%, | A CoV S glycoprotein with at least 90%, |
| | at least 91%, at least 92%, at least | at least 91%, at least 92%, at least |
| | 93%, at least 94%, at least 95%, at | 93%, at least 94%, at least 95%, at |
| | least 96%, at least 97%, at least 98%, | least 96%, at least 97%, at least 98%, |
| | at least 99%, or 100% identity to any | at least 99%, or 100% identity to |
| | one of SEQ ID NO: 245, SEQ ID NO: | any one of SEQ ID NOS: 246-249, |
| | 250, SEQ ID NO: 255, and SEQ ID | 251-254, 256-259, and 261-264 |
| | NO: 260 | |
| 2 | A CoV S glycoprotein with at least 90%, | A CoV S glycoprotein with at least 90%, |
| | at least 91%, at least 92%, at least | at least 91%, at least 92%, at least |
| | 93%, at least 94%, at least 95%, at | 93%, at least 94%, at least 95%, at |
| | least 96%, at least 97%, at least 98%, | least 96%, at least 97%, at least 98%, |
| | at least 99%, or 100% identity to any | at least 99%, or 100% identity to |
| | one of SEQ ID NO: 265, SEQ ID NO: | any one of SEQ ID NOS: 267-272, |
| | 266, SEQ ID NO: 273, and SEQ ID | 275-280 |
| | NO: 274 | |
| 3 | A CoV S glycoprotein with at least 90%, | A CoV S glycoprotein with at least 90%, |
| | at least 91%, at least 92%, at least | at least 91%, at least 92%, at least |
| | 93%, at least 94%, at least 95%, at | 93%, at least 94%, at least 95%, at |
| | least 96%, at least 97%, at least 98%, | least 96%, at least 97%, at least 98%, |
| | at least 99%, or 100% identity to any | at least 99%, or 100% identity to |
| | one of SEQ ID NOS: 281-284 | any one of SEQ ID NOS: 285-292 |
| 4 | A CoV S glycoprotein with at least 90%, | A CoV S glycoprotein with at least 90%, |
| | at least 91%, at least 92%, at least | at least 91%, at least 92%, at least |
| | 93%, at least 94%, at least 95%, at | 93%, at least 94%, at least 95%, at |
| | least 96%, at least 97%, at least 98%, | least 96%, at least 97%, at least 98%, |
| | at least 99%, or 100% identity to any | at least 99%, or 100% identity to |
| | one of SEQ ID NO: 245, SEQ ID NO: | any one of SEQ ID NOS: 246-249, |
| | 250, SEQ ID NO: 255, and SEQ ID | 251-254, 256-259, 261-264, 267-272, |
| | NO: 260 | 275-280, and 285-292 |
| 5 | A CoV S glycoprotein with at least 90%, | A CoV S glycoprotein with at least 90%, |
| | at least 91%, at least 92%, at least | at least 91%, at least 92%, at least |
| | 93%, at least 94%, at least 95%, at | 93%, at least 94%, at least 95%, at |
| | least 96%, at least 97%, at least 98%, | least 96%, at least 97%, at least 98%, |
| | at least 99%, or 100% identity to any | at least 99%, or 100% identity to |
| | one of SEQ ID NO: 265, SEQ ID NO: | any one of SEQ ID NOS: 246-249, |
| | 266, SEQ ID NO: 273, and SEQ ID | 251-254, 256-259, 261-264, 267-272, |
| | NO: 274 | 275-280, and 285-292 |
| 6 | A CoV S glycoprotein with at least 90%, | A CoV S glycoprotein with at least 90%, |
| | at least 91%, at least 92%, at least | at least 91%, at least 92%, at least |
| | 93%, at least 94%, at least 95%, at | 93%, at least 94%, at least 95%, at |
| | least 96%, at least 97%, at least 98%, | least 96%, at least 97%, at least 98%, |
| | at least 99%, or 100% identity to any | at least 99%, or 100% identity to |
| | one of SEQ ID NOS: 281-284 | any one of SEQ ID NOS: 246-249, |
| | | 251-254, 256-259, 261-264, 267-272, |
| | | 275-280, and 285-292 |

The five CoV S polypeptide nanoparticles are produced according to the procedures of Example 1 and suspended in a pharmaceutically acceptable buffer to form an immunogenic composition. An adjuvant may be added to the composition. The adjuvant may be a saponin adjuvant. For example, the saponin adjuvant may contain two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. Fraction A and Fraction C account for 85% and 15% by weight, respectively, of the sum of the weights of fraction A of Quillaja Saponaria Molina and fraction C of Quillaja Saponaria Molina in the adjuvant.

TABLE 2C2
CoV S Polypeptides

| | | Modification (numbered according |
|---|---|---|
| CoV S polypeptide | SEQ ID NO. | to SEQ ID NO: 1) |
| 1A | 245 | |
| 1B | 246 | P521S |
| 1C | 247 | K444T |
| 1D | 248 | F486S |
| 1E | 249 | T478K |
| 1A-1 | 250 | |
| 1B-1 | 251 | P521S |
| 1C-1 | 252 | K444T |
| 1D-1 | 253 | F486S |
| 1E-1 | 254 | T478K |
| 1A-2 | 255 | |
| 1B-2 | 256 | P521S |
| 1C-2 | 257 | K444T |
| 1D-2 | 258 | F486S |
| 1E-2 | 259 | T478K |
| 1A-3 | 260 | |
| 1B-3 | 261 | P521S |
| 1C-3 | 262 | K444T |
| 1D-3 | 263 | F486S |
| 1E-3 | 264 | T478K |
| 2A | 265 | |
| 2B | 266 | Inactive primary furin cleavage site, |
| | | mutation of amino acids 986 and 987 |
| | | to proline |
| 2C | 267 | P521S |
| 2D | 268 | Inactive primary furin cleavage site, |
| | | mutation of amino acids 986 and 987 |
| | | to proline, P521S |
| 2A-1 | 269 | K444T |
| 2B-1 | 270 | Inactive primary furin cleavage site, |
| | | mutation of amino acids 986 and 987 |
| | | to proline, K444T |
| 2C-1 | 271 | F486S |
| 2D-1 | 272 | Inactive primary furin cleavage site, |
| | | mutation of amino acids 986 and 987 |
| | | to proline, K444T, F486S |
| 2A-2 | 273 | |
| 2B-2 | 274 | Inactive primary furin cleavage site, |
| | | mutation of amino acids 986 and 987 |
| | | to proline |
| 2C-2 | 275 | P521S |
| 2D-2 | 276 | Inactive primary furin cleavage site, |
| | | mutation of amino acids 986 and 987 |
| | | to proline, P521S |
| 2A-3 | 277 | K444T |
| 2B-3 | 278 | Inactive primary furin cleavage site, |
| | | mutation of amino acids 986 and 987 |
| | | to proline, K444T |
| 2C-3 | 279 | F486S |
| 2D-3 | 280 | Inactive primary furin cleavage site, |
| | | mutation of amino acids 986 and 987 |
| | | to proline, K444T, F486S |
| 3A-1 | 281 | |
| 3A-2 | 285 | K444T |
| 3A-3 | 289 | T486S |
| 3B-1 | 282 | Inactive primary furin cleavage site, |
| | | mutation of amino acids 986 and 987 |
| | | to proline |
| 3B-2 | 286 | Inactive primary furin cleavage site, |
| | | mutation of amino acids 986 and 987 |
| | | to proline, K444T |
| 3B-3 | 290 | Inactive primary furin cleavage site, |
| | | mutation of amino acids 986 and 987 |
| | | to proline, T486S |
| 3C-1 | 283 | |
| 3C-2 | 287 | K444T |
| 3C-3 | 291 | T486S |
| 3D-1 | 284 | Inactive primary furin cleavage site, |
| | | mutation of amino acids 986 and 987 |
| | | to proline |
| 3D-2 | 288 | Inactive primary furin cleavage site, |
| | | mutation of amino acids 986 and 987 |
| | | to proline, K444T |
| 3D-3 | 292 | Inactive primary furin cleavage site, |
| | | mutation of amino acids 986 and 987 |
| | | to proline, T486S |

### Example 3

### Immunogenicity of Monovalent and Bivalent COVID-19 Immunogenic Compositions

Monovalent COVID-19 immunogenic compositions were prepared that contained one COV S glycoprotein. Bivalent COVID-19 immunogenic compositions were also prepared which had two CoV S glycoproteins. The immunogenicity of the immunogenic compositions below was evaluated. Table C3 shows the identity of the CoV S glycoproteins in each composition.

TABLE C3

| | Amino acid sequence of CoV S |
|---|---|
| Composition | glycoprotein(s) in composition |
| 1 (also referred to as “XBB.1.16”) | SEQ ID NO: 260 |
| 2 (also referred to as (“XBB.1.5”) | SEQ ID NO: 274 |
| 3 (also referred to as (“Prototype”) | SEQ ID NO: 87 |
| 4 (also referred to as (“Prototype + | SEQ ID NO: 87 and |
| XBB.1.5”) | SEQ ID NO: 274 |
| 5 (also referred to as “Prototype + | SEQ ID NO: 87 and |
| BA.5”) | SEQ ID NO: 222 |
| 6 (also referred to as “BA.5”) | SEQ ID NO: 222 |

The CoV S glycoprotein nanoparticles were produced according to the procedures of Example 1 and suspended in a pharmaceutically acceptable buffer to form an immunogenic composition. Each composition contained a saponin adjuvant that contained two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. Fraction A and Fraction C account for 85% and 15% by weight, respectively, of the sum of the weights of fraction A of Quillaja Saponaria Molina and fraction C of Quillaja Saponaria Molina in the adjuvant.

### Administration of Monovalent Compositions

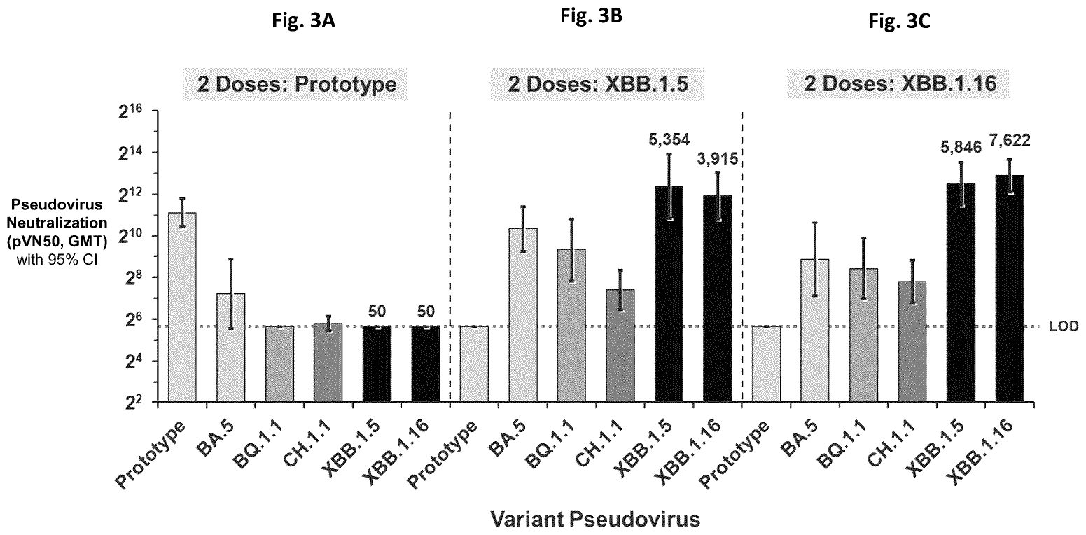

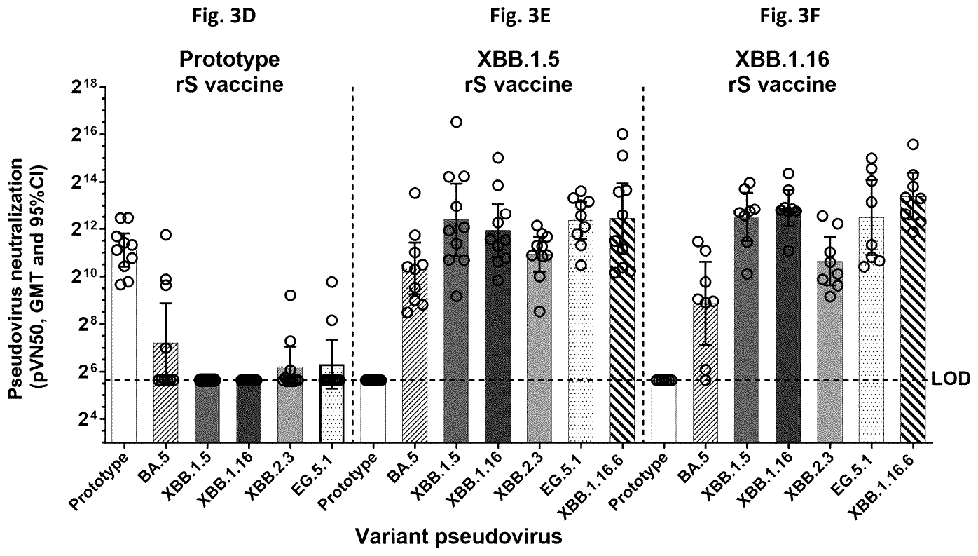

Groups of mice (n=10) were inoculated intramuscularly with two doses of Composition 3, two doses of Composition 2, or two doses of Composition 1. FIGS. 3A-3C shows that Composition 3 (FIG. 3A, FIG. 3D), Composition 2 (FIG. 3B, FIG. 3E), and Composition 1 (FIG. 3C, FIG. 3F) each neutralized pseudoviruses expressing S proteins from various SARS-COV-2 heterogeneous strains. Humoral responses following administration of Composition 2 and Composition 1 were comparable and highly immunogenic as they induced pseudovirus neutralizing antibodies against SARS-COV-2 Omicron BA.5, XBB.1.5, XBB.1.16, XBB.2.3, EG.5.1, and XBB.1.16.6. (FIG. 3E, FIG. 3F). Following the primary vaccination series, homologous variant responses for Composition 2 (GMT-4148, 95% CI: 2494-6900) and Composition 1 (GMT=7622, 95% CI: 4451-13053) vaccinations were slightly higher than heterologous responses for Composition 2 to a pseudovirus expressing the SARS-COV-2 S glycoprotein of Composition 1 (GMT=3473, 95% CI: 2282-5287) and Composition 1 to a SARS-COV-2 pseudovirus expressing the SARS-COV-2 S glycoprotein of Composition 2 (GMT=5846, 95% CI: 2863-11940) (FIG. 3E, FIG. 3F). Compared to homologous variant responses, EG.5.1 neutralization responses were similar in magnitude following immunization with XBB.1.5 (GMT=6478, 95% CI: 3464-12112) and XBB.1.16 (GMT=5770, 95% CI: 1918-17358). (FIG. 3E). XBB.1.16.6 variant neutralization responses were also similar following immunization with XBB.1.5 (GMT=5548, 95% CI: 1960-15704), and XBB.1.16 (GMT=10781, 95% CI: 5413-21472) (FIG. 3E). The magnitude of EG.5.1 and XBB subvariant responses were comparable to the levels achieved with homologous responses to a two-dose primary series of Prototype vaccine, suggesting adequate antibody levels were achieved.

### Immunogenicity Induced by Bivalent Composition 4 Compared to Monovalent Composition 2

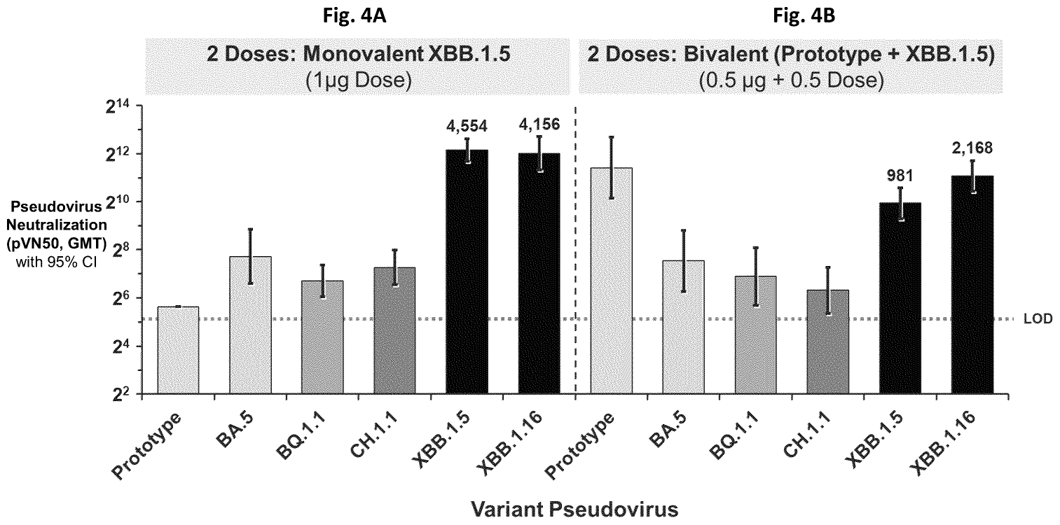

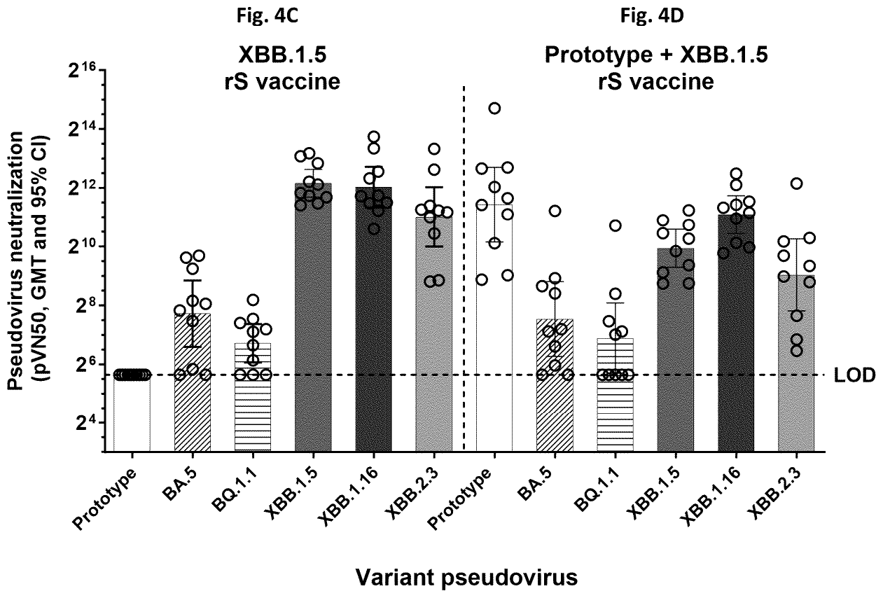

Mice (n=10 per group) were inoculated intramuscularly with Composition 2 (1 μg) or Composition 4, a bivalent composition containing 0.5 μg Prototype CoV S glycoprotein (SEQ ID NO: 87) and 0.5 μg XBB.1.5 (SEQ ID NO: 274), on days 0 and 14, and sera were collected at day 21 (1 week after the second dose). FIGS. 4A-4B show that administration of two doses of Composition 2 (FIG. 4A, FIG. 4C), a monovalent composition comprising a CoV S glycoprotein of SEQ ID NO: 274, results in a superior immune response in mice to administration of two doses of Composition 4 (FIG. 4B, FIG. 4D), the bivalent composition containing a CoV S glycoprotein of SEQ ID NO: 274 and a CoV S glycoprotein of SEQ ID NO: 87. Specifically, the monovalent composition elicited cross-reactive pseudovirus neutralizing antibodies against the SARS-COV-2 Variant Strains, Omicron XBB.1.5 (GMT=4554, 95% CI: 3285-6313), XBB.1.16 (GMT=4156, 95% CI: 2568-6724), and XBB.2.3 (GMT=2058, 95% CI: 1019-4157). In contrast, the bivalent composition induced a ˜50% or lower response to XBB.1.5 (GMT=981, 95% CI: 625-1541), XBB.1.16 (GMT=2168, 95% CI: 1387-3389), and XBB.2.3 (GMT=524, 95% CI: 224-1227)

### Effect of Administering Composition 5 as Primary Series and Composition 2 or Composition 1 Boosters on Immunogenicity Against SARS-COV-2 Variants

Groups of mice (n=10 per group) were inoculated intramuscularly with a primary immunization series of a bivalent vaccine (Composition 5) on days 0 and 14, followed by a single booster dose with either Composition 2 or Composition 1 on day 47. Sera were collected at day 21 (1 week after the second dose) and day 61 (2 weeks after booster dose).

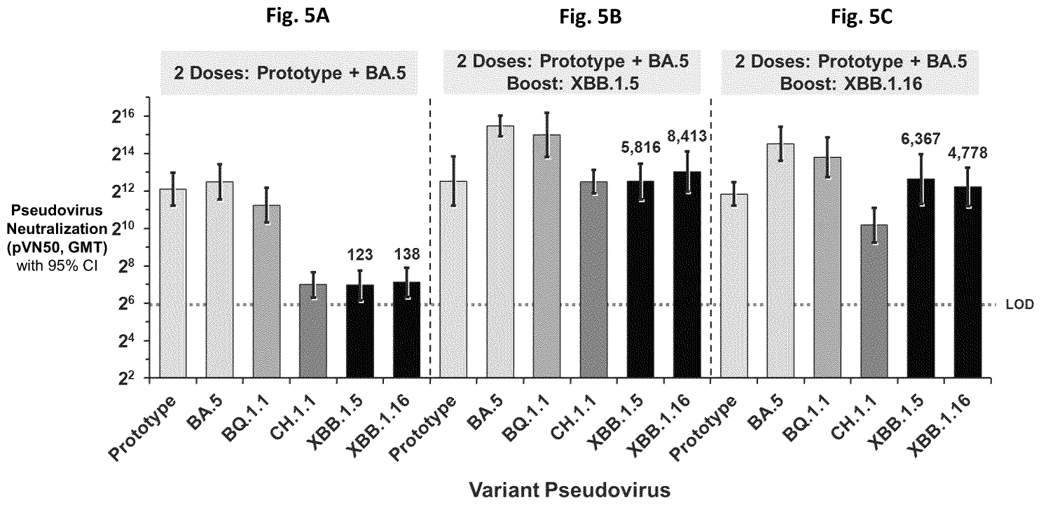

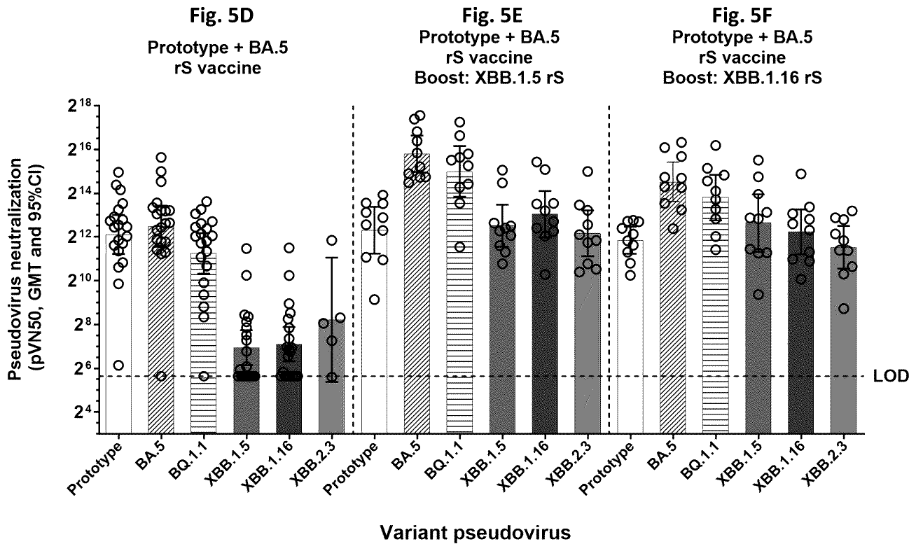

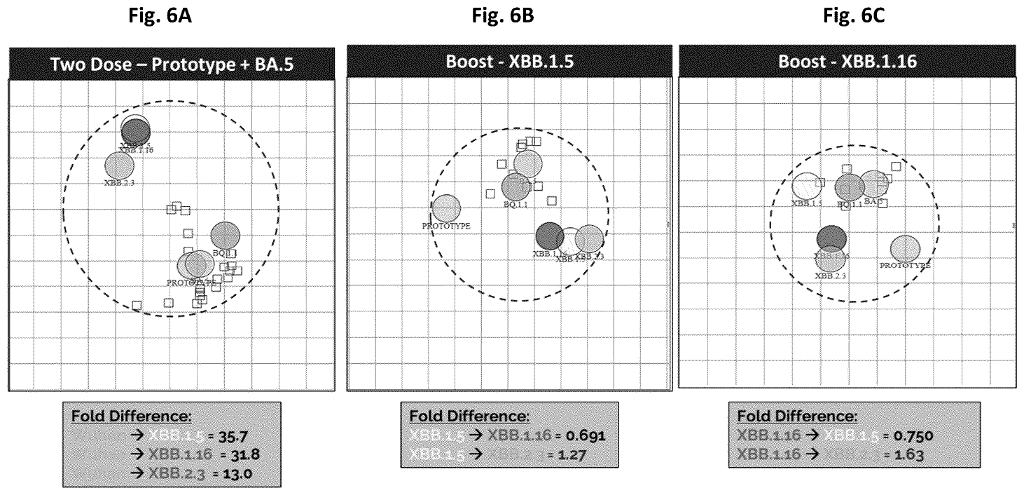

FIGS. 5A-5C and FIGS. 6A-6C shows the immune response induced by a boost dose containing Composition 2 (FIG. 5B FIG. 6B, FIG. 5E) or Composition 1 (FIG. 5C, FIG. 5F, FIG. 6C). Boosting with either Composition 2 (FIG. 5B, FIG. 6B, FIG. 5E) or Composition 1 (FIG. 5C, FIG. 5F, FIG. 6C) resulted in an improved immune response against pseudoviruses expressing S proteins from various SARS-COV-2 heterogeneous strains, as compared to mice that were not boosted (FIG. 5A, FIG. 5D, FIG. 6A). Specifically, boosting with Composition 2 or Composition 1 induced a >35-fold increase in pseudovirus neutralizing antibodies against SARS-COV-2 pseudoviruses expressing the SARS-COV-2 S glycoprotein of Composition 2 or Composition 1 (FIGS. 5D-5F). Pseudovirus neutralizing antibodies were higher against XBB.2.3 following immunization with Composition 2 (GMT=4561, 95% CI: 2219-9375) compared to levels after immunization with Composition 1 (GMT=2938, 95% CI: 1495-5774).

Pseudovirus neutralizing antibody responses in mice were further analyzed by antigenic cartography. Priming with Composition 5 resulted in an immune response to Omicron XBB.1.5 and XBB.1.16 variants that was 30-fold greater than priming with Composition 3 (FIG. 6A). Boosting primed mice with Composition 2 induced a matched response to XBB.1.16, with an antigenic distance of 0.691 (FIG. 6B). Similarly, a boost with Composition 1 induced a matched response to XBB.1.5 with a fold-difference of 0.750 (FIG. 6C). Antigenic distances with a fold-difference less than 2-fold are considered to be matched responses.

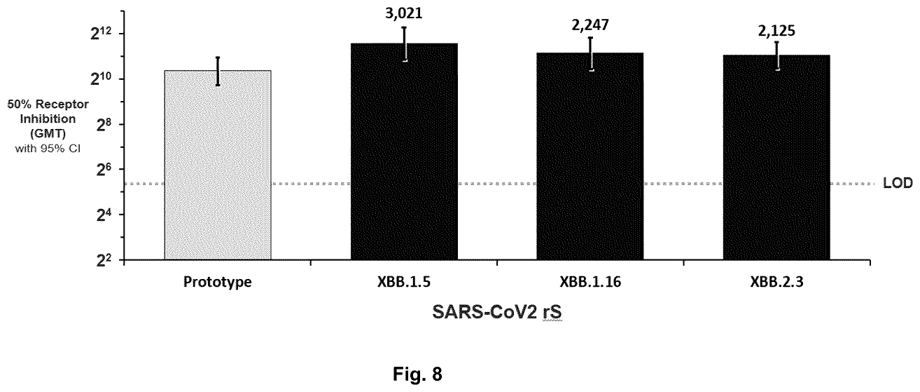

FIG. 8 shows the ability of a dosage regimen comprising administering two doses of Composition 5 followed by boosting with Composition 2 to induce an immune response that blocks binding of hACE 2 to a SARS-COV-2 S glycoprotein.

### CD4+ T Cell Responses in Mice:

Mice were administered two doses of Composition 3, which contains a CoV S glycoprotein of SEQ ID NO: 87 or two doses of Composition 5, which comprises a CoV S glycoprotein of SEQ ID NO: 87 and a CoV S glycoprotein of SEQ ID NO: 222. The mice were boosted with Composition 2.

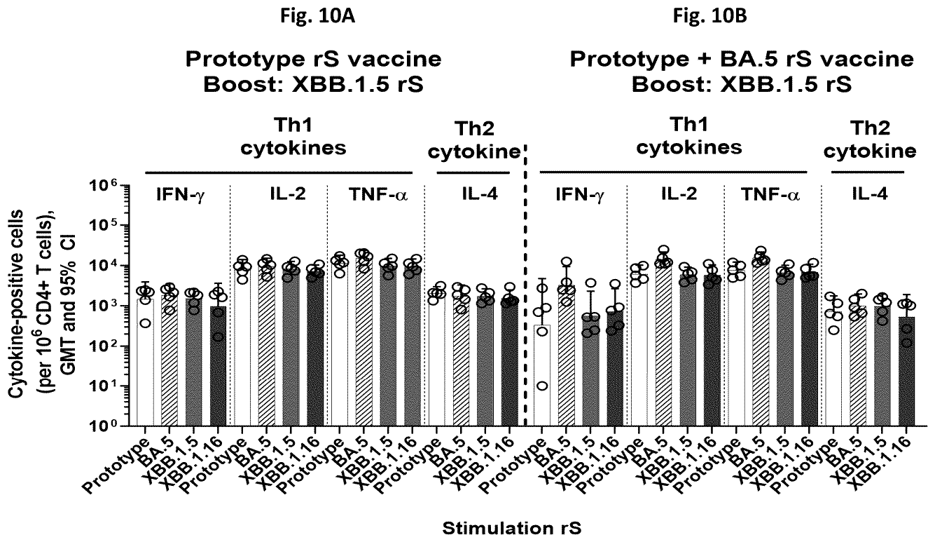

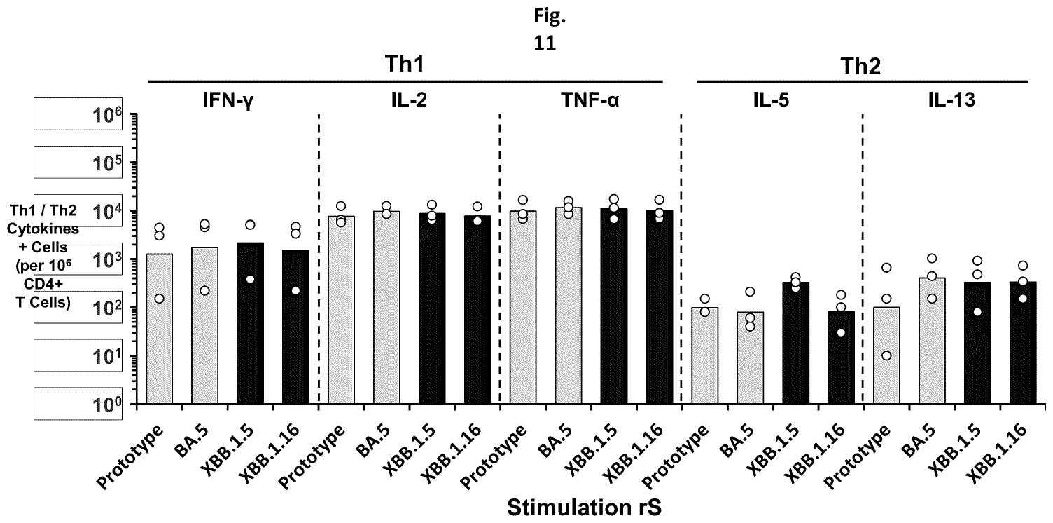

Th1 (IFN-γ, IL-2, and TNF-α) and Th2 (IL-4) cytokine-producing CD4+ T cells were measured in splenocytes isolated two weeks post booster dose. A robust CD4+ T cell response was recalled at comparable levels post boost upon boosting with Composition 2, irrespective of priming vaccine (i.e., Composition 3 or Composition 5) (FIG. 10A, FIG. 10B).

Results from Primary Vaccine Regimen Followed by Boosting in Rhesus Macaques:

Rhesus macaques (Macaca mulatta; n=5 per group) were administered two doses of Composition 3, which contains a CoV S glycoprotein of SEQ ID NO: 87, two doses of Composition 6, which comprises a CoV S glycoprotein of SEQ ID NO: 222, or two doses of Composition 5, which comprises a CoV S glycoprotein of SEQ ID NO: 87 and a CoV S glycoprotein of SEQ ID NO: 222 on days 0 and 21. These doses are “priming” doses. The macaques were boosted with Composition 2 on day 246, which contains a CoV S glycoprotein of SEQ ID NO: 274. All macaques were monitored twice daily to determine the safety of the vaccines, and no local or systemic side effects were reported after the primary series or booster vaccinations.

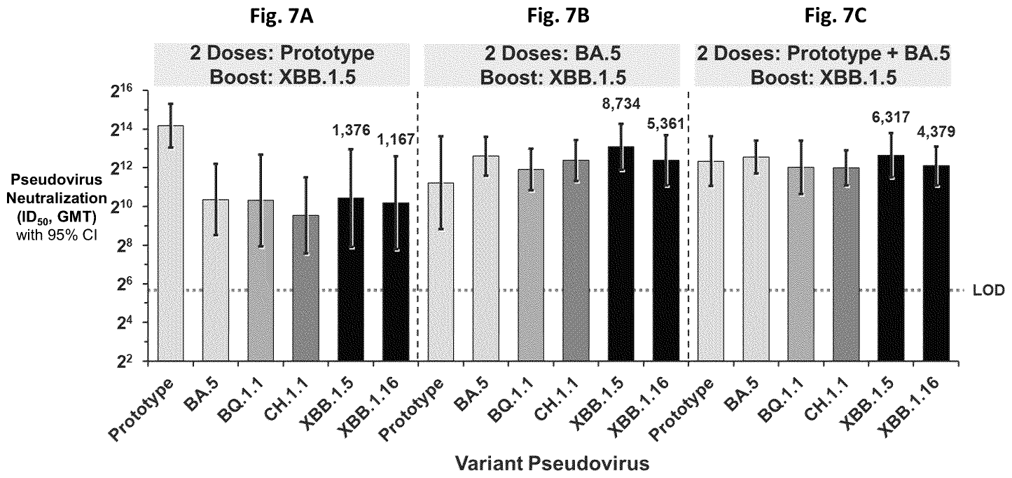

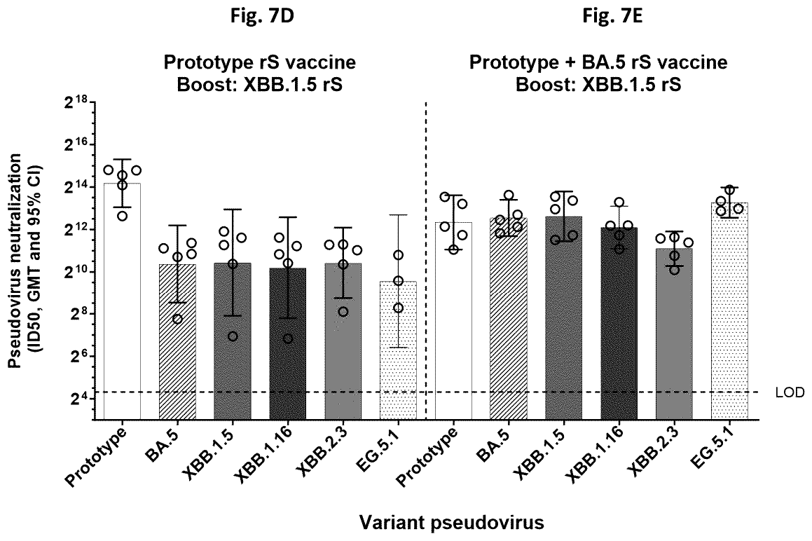

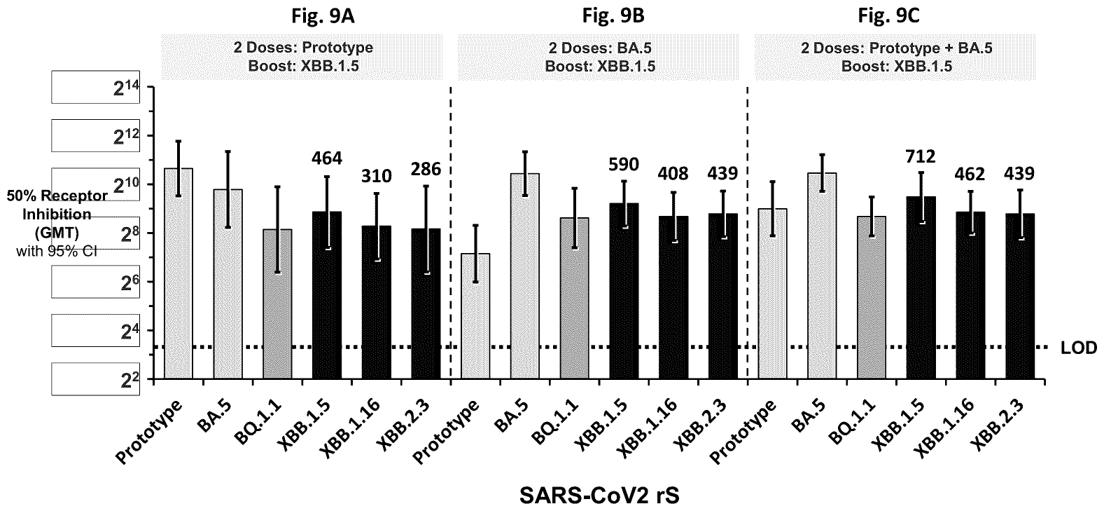

Boosting with Composition 2 resulted in comparable neutralizing responses to pseudoviruses expressing XBB1.5, XBB 1.16 S, XBB.2.3, and EG.5.1 glycoproteins. Priming with a composition containing a CoV S glycoprotein of SEQ ID NO: 222 (Compositions 5 and 6) resulted in superior neutralization compared to priming with compositions lacking a CoV S glycoprotein of SEQ ID NO: 222 (Composition 3) (FIGS. 7A-7E). Specifically, priming with a composition containing a CoV S glycoprotein of SEQ ID NO: 222 (e.g., Compositions 5 and 6) resulted in higher neutralizing antibody titers against Omicron XBB variants than priming with Composition 3. Boosting with Composition 2 also resulted in the induction of an immune response that blocks the interaction between hACE2 and the CoV S glycoprotein. (FIGS. 9A-9C). FIG. 11 shows the CD4+ T cell response in Rhesus macaques administered two doses of Composition 5, followed by a boost dose of Composition 2. Macaques primed with bivalent vaccine (Composition 5) and boosted with XBB.1.5 (Composition 2) elicited a Th1-biased cellular response with comparable magnitudes of cytokine-positive cells for all variants tested (FIG. 11).

### Example 4

### Immunogenicity of Immunogenic Compositions of Example 2 and 3 in Mice

The immunogenic compositions described in Examples 2 and 3 are evaluated for immunogenicity and toxicity in a murine model, using female BALB/c mice (7-9 weeks old; Harlan Laboratories Inc., Frederick, MD). The compositions are evaluated in the presence of an adjuvant (e.g., the saponin adjuvant of Example 2). The compositions may contain from 1 μg to about 200 μg of adjuvant and from 0.01 μg of CoV S polypeptide to about 100 μg of CoV S polypeptide. Immunogenic compositions are administered intramuscularly as a single dose (also referred to as a single priming dose) (study day 14) or as two doses (also referred to as a prime/boost regimen) spaced 14 to 21-days apart. A placebo group served as a non-immunized control. Serum is collected for analysis on study days 1, 13, 21, 28, and 35. Serum is analyzed for the presence of anti-Spike antibodies.

To evaluate the induction of protective immunity, immunized mice are challenged with SARS-COV-2. Since mice do not support replication of the wild-type SARS-COV-2 virus, on day 52 post initial vaccination, mice are intranasally infected with an adenovirus expressing hACE2 (Ad/hACE2) to render them permissive. Immunized and control animals are intranasally challenged with SARS-COV-2 42 days following one (a single dose) or two (two doses) immunizations. Challenged mice are weighed on the day of infection and daily for up to 7 days post infection. At 4- and 7-days post infection, 5 mice are sacrificed from each vaccination and control group, and lungs were harvested and prepared for pulmonary histology.

The viral titer is quantified by a plaque assay. Briefly, the harvested lungs are homogenized in PBS using 1.0 mm glass beads (Sigma Aldrich) and a Beadruptor (Omini International Inc.). Homogenates were added to Vero E6 near confluent cultures and SARS-COV-2 virus titers determined by counting plaque forming units (pfu) using a 6-point dilution curve.

The effect of the immunogenic compositions on the induction of cellular immunity is also evaluated. Spleens are collected 7-days after the second immunization (study day 28). A non-vaccinated group (N=3) served as a control.

### T Cell Response

The effect of the immunogenic compositions on the T cell response are evaluated. Antigen-specific T cell responses are measured by ELISPOT™ enzyme linked immunosorbent assay and intracellular cytokine staining (ICCS) from spleens collected 7-days after the second immunization (study day 28). The frequency of IFN-γ+, TNF-α+, and IL-2+ cytokine-secreting CD4+ and CD8+ T cells is evaluated. Type 2 cytokine IL-4 and IL-5 secretion from CD4+ T cells is also determined by ICCS and ELISPOT™ respectively. Finally, the effect of immunization on germinal center formation is assessed by measuring the frequency of CD4+ T follicular helper (TFH) cells and germinal center (GC) B cells in spleens.

### Example 5

### Immunogenicity of Immunogenic Composition of Examples 2 and 3 in Olive Baboons and Cynomolgous Macaques

The immunogenicity of the immunogenic compositions of Examples 2 and 3 is evaluated in baboons and cynomolgus macaques. Adult olive baboons and/or cynomolgus macaques are immunized with any one of the immunogenic compositions of Example 2 and/or 3 with and without adjuvant in two doses spaced 21-days apart. Anti-S protein IgG titers are measured every day after immunization. The level of hACE2 receptor blocking antibodies is also evaluated.

The effect of the immunogenic composition on cellular immunity is also evaluated. PBMCs are collected 7 days after the second immunization (day 28), and the T cell response is measured by ELISPOT assay.

### Example 6

### Immunogenicity of Immunogenic Composition of Examples 2 and 3 in Humans

The safety and immunogenicity of the immunogenic compositions of Examples 2 and 3 are assessed in a Phase ½ study in adults. The first CoV S glycoprotein is present in the composition at about 3 μg to about 25 μg. The second, third, fourth, and fifth CoV S glycoproteins are present in the composition in smaller amounts. The total amount of CoV S glycoprotein evaluated is 5, 10, and 25 μg. The immunogenicity of the compositions is evaluated in the presence and absence of an adjuvant (e.g., the saponin adjuvant of Example 2A and 2B). About 50 μg adjuvant is present in the composition.

### Example 7

### Immunogenicity of Immunogenic Composition of Examples 2 and 3 in Mice

The pentavalent immunogenic compositions described in Example 2 and the bivalent and monovalent immunogenic compositions of Example 1 are evaluated for immunogenicity and toxicity in a BALB/c mouse model (10 per group). The compositions contain 5 μg saponin adjuvant. The compositions contain about 1 μg of S protein. As a control, a composition containing 1 μg of one CoV S glycoprotein and 5 μg saponin adjuvant is administered to the mice.

The saponin adjuvant contains two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. Fraction A and Fraction C account for 85% and 15% by weight, respectively, of the sum of the weights of fraction A of Quillaja Saponaria Molina and fraction C of Quillaja Saponaria Molina in the adjuvant.

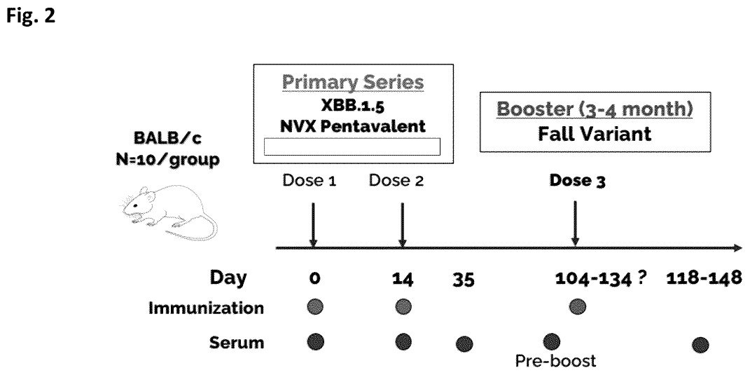

Three doses of the immunogenic compositions are administered to the mice. The first doses is administered on day 0. The second dose is administered 14 days after administration of the second dose (day 14). The third dose is administered 3-4 months after administration of the first dose (from day 104 to day 134). FIG. 2 shows a schematic of the dosage regimen.

Serum is collected from the mice on days 0, 14, 35, between days 35 and 104-134, and on one day from days 118 to 148.

Serum is analyzed for the presence of anti-Spike antibodies.

The ability of the serum to neutralize pseudovirus expressing CoV S glycoproteins is also evaluated.

To evaluate the induction of protective immunity, immunized mice are challenged with SARS-COV-2. Since mice do not support replication of the wild-type SARS-COV-2 virus, on day 52 post initial vaccination, mice are intranasally infected with an adenovirus expressing hACE2 (Ad/hACE2) to render them permissive. Immunized and control animals are intranasally challenged with SARS-COV-2 42 days following one (a single dose) or two (two doses) immunizations. Challenged mice are weighed on the day of infection and daily for up to 7 days post infection. At 4- and 7-days post infection, 5 mice are sacrificed from each vaccination and control group, and lungs were harvested and prepared for pulmonary histology.

The viral titer is quantified by a plaque assay. Briefly, the harvested lungs are homogenized in PBS using 1.0 mm glass beads (Sigma Aldrich) and a Beadruptor (Omini International Inc.). Homogenates were added to Vero E6 near confluent cultures and SARS-COV-2 virus titers determined by counting plaque forming units (pfu) using a 6-point dilution curve.

The effect of the immunogenic compositions on the induction of cellular immunity is also evaluated. Spleens are collected 7-days after the second immunization (study day 28). A non-vaccinated group (N=3) served as a control.

### T Cell Response

The effect of compositions on the T cell response is evaluated. Antigen-specific T cell responses are measured by ELISPOT™ enzyme linked immunosorbent assay and intracellular cytokine staining (ICCS) from spleens collected 7-days after the second immunization (study day 28). The frequency of IFN-γ+, TNF-α+, and IL-2+ cytokine-secreting CD4+ and CD8+ T cells is evaluated. Type 2 cytokine IL-4 and IL-5 secretion from CD4+ T cells is also determined by ICCS and ELISPOT™ respectively. Finally, the effect of immunization on germinal center formation is assessed by measuring the frequency of CD4+ T follicular helper (TFH) cells and germinal center (GC) B cells in spleens.

### Example 8

### Immunogenicity of Immunogenic Compositions in Mice

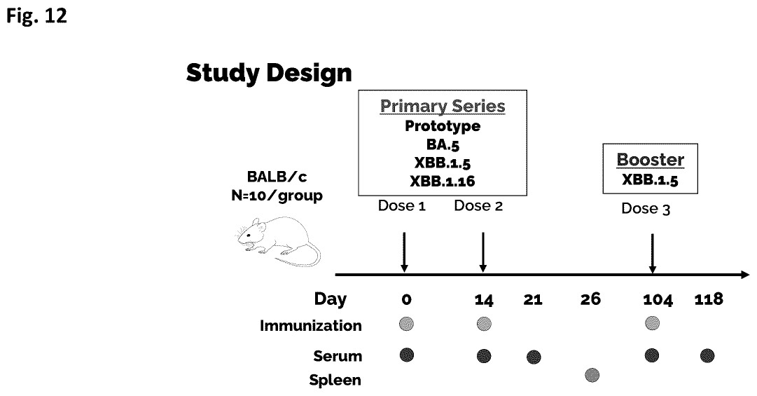

Immunogenic compositions comprising a SARS-COV-2 S glycoprotein and a saponin adjuvant were administered to BALB/c mice (n=10). The saponin adjuvant contained two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. Fraction A and Fraction C account for 85% and 15% by weight, respectively, of the sum of the weights of fraction A of Quillaja Saponaria Molina and fraction C of Quillaja Saponaria Molina in the adjuvant. The SARS-COV-2 S glycoproteins were (i) a SARS-COV-2 S glycoprotein of SEQ ID NO: 260 (referred to as XBB.1.16), (ii) a SARS-COV-2 S glycoprotein of SEQ ID NO: 274 (referred to as XBB.1.5), (iii) a SARS-COV-2 S glycoprotein of SEQ ID NO: 87 (referred to as prototype), or (iv) a SARS-COV-2 S glycoprotein of SEQ ID NO: 284 (referred to as XBB2.3). The immunogenic compositions were administered according to the schedule in FIG. 12. Each immunogenic composition contained 1 μg SARS-COV-2 S glycoprotein and 5 μg of saponin adjuvant.

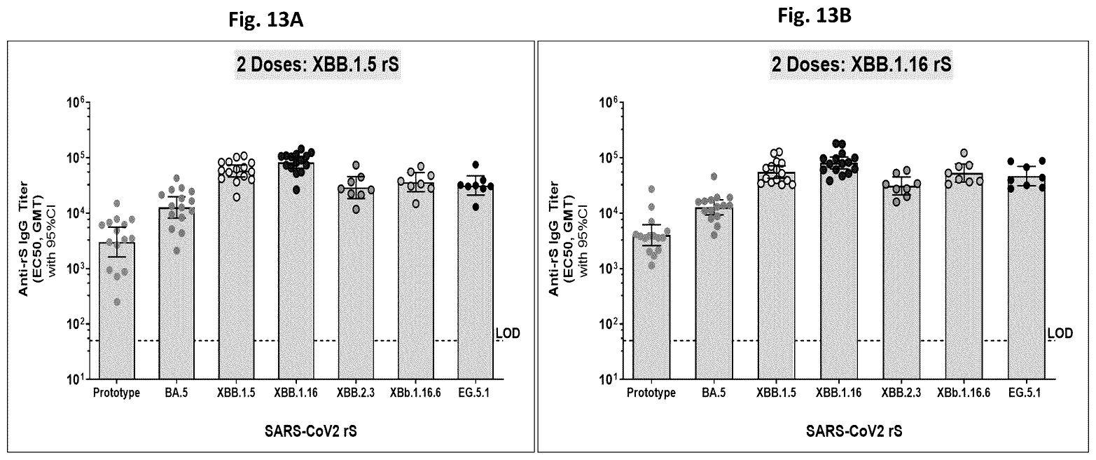

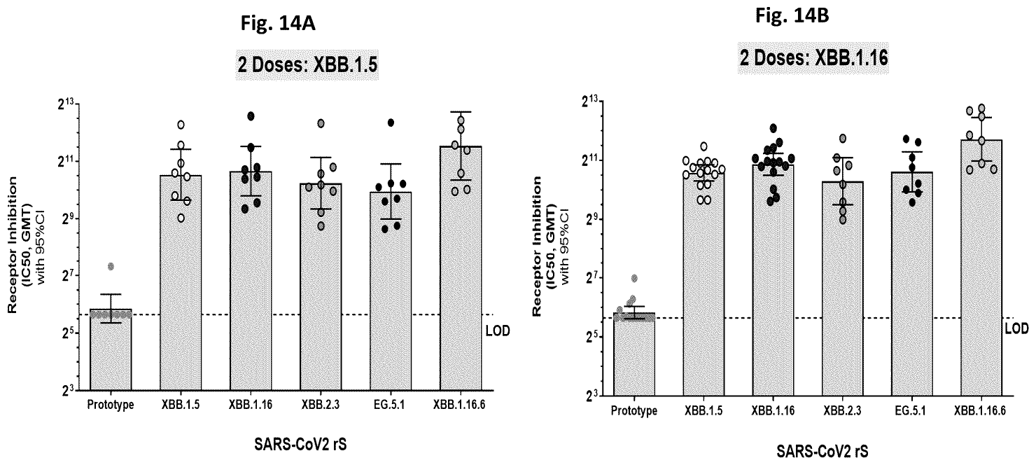

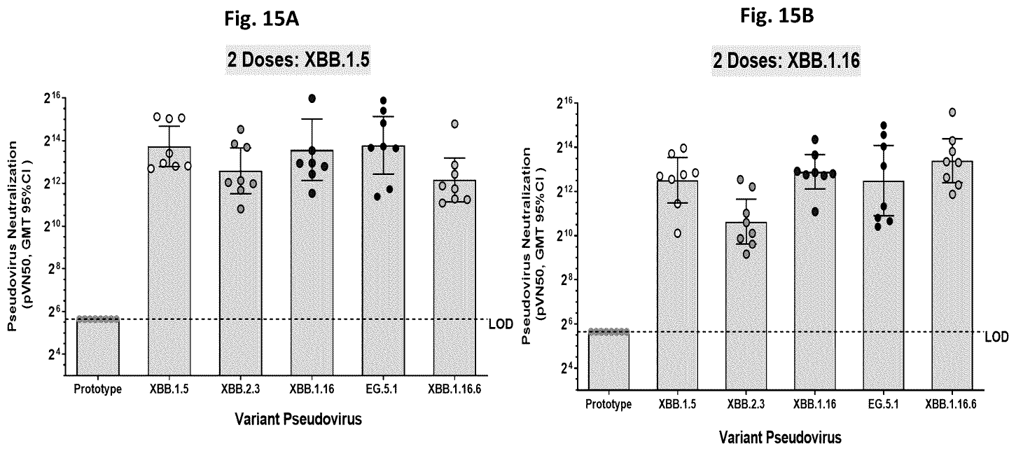

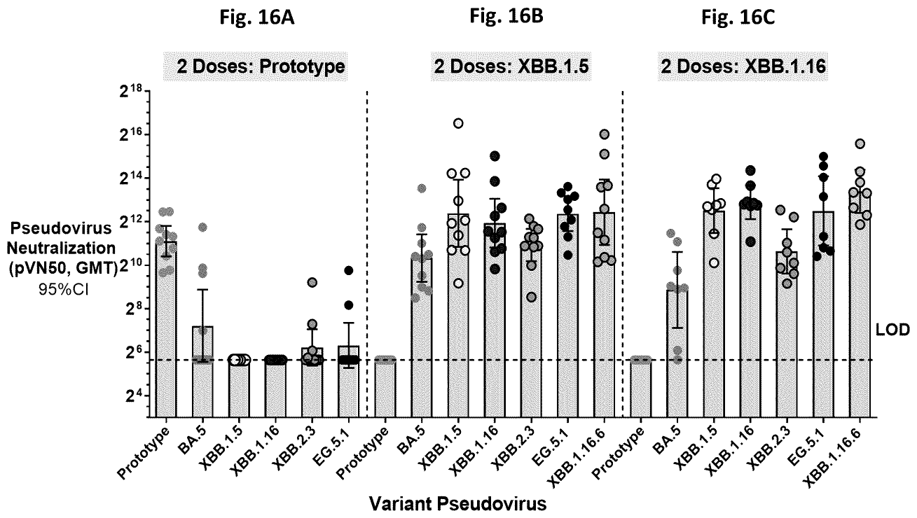

FIG. 13A shows the anti-S IgG antibodies in mice after administration of two doses of an immunogenic composition comprising the SARS-COV 2 S glycoprotein of SEQ ID NO: 274. FIG. 13B shows the anti-S IgG antibodies in mice after administration of two doses of an immunogenic composition comprising the SARS-COV 2 S glycoprotein of SEQ ID NO: 260. FIG. 14A shows that administration of two doses of an immunogenic composition comprising the SARS-COV 2 S glycoprotein of SEQ ID NO: 274 results in antibodies that block binding of SARS-COV-2 to hACE2. FIG. 14B shows that administration of two doses of an immunogenic composition comprising the SARS-COV 2 S glycoprotein of SEQ ID NO: 260 results in antibodies that block binding of SARS-COV-2 to hACE2. FIG. 15A and FIG. 16B shows that administration of two doses of an immunogenic composition comprising the SARS-COV 2 S glycoprotein of SEQ ID NO: 274 results in neutralization of pseudoviruses expressing SARS-COV-2 S glycoproteins. FIG. 15B and FIG. 16C shows that administration of two doses of an immunogenic composition comprising the SARS-COV 2 S glycoprotein of SEQ ID NO: 260 results in neutralization of pseudoviruses expressing SARS-COV-2 S glycoproteins. FIG. 16A shows the neutralizing antibody titer resulting from administering two doses of an immunogenic composition comprising the SARS-COV 2 S glycoprotein of SEQ ID NO: 87.

### Example 9

### Immunogenicity of Immunogenic Compositions in Mice

Immunogenic compositions comprising one or two SARS-COV-2 S glycoproteins and a saponin adjuvant were administered to BALB/c mice (n=10). The saponin adjuvant contained two iscom particles, wherein: the first iscom particle comprises fraction A of Quillaja Saponaria

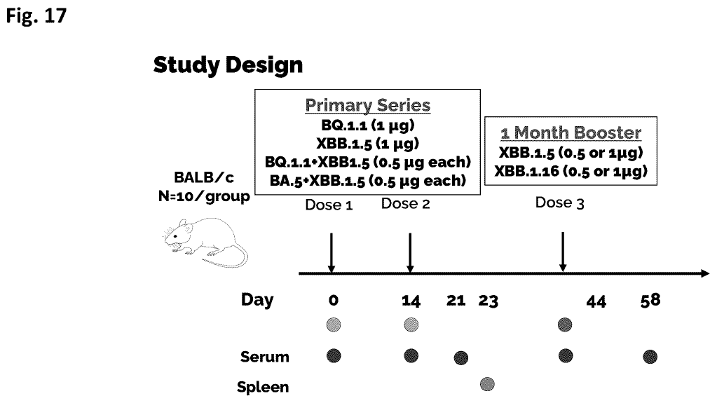

Molina and not fraction C of Quillaja Saponaria Molina; and the second iscom particle comprises fraction C of Quillaja Saponaria Molina and not fraction A of Quillaja Saponaria Molina. Fraction A and Fraction C account for 85% and 15% by weight, respectively, of the sum of the weights of fraction A of Quillaja Saponaria Molina and fraction C of Quillaja Saponaria Molina in the adjuvant. The immunogenic compositions were administered according to the schedule in FIG. 17. The mice were administered a first dose and second dose of a first immunogenic composition on day 0 and day 14 (referred to as the “primary series.” The mice were administered a boost dose of a second immunogenic composition on day 44 (referred to as “1 month booster”).

Each first immunogenic composition contained 1 μg total SARS-COV-2 S glycoprotein and 5 μg saponin adjuvant. When two glycoproteins were present in the first immunogenic composition, each glycoprotein was present in the composition in equal amounts. The first compositions contained a saponin adjuvant and (i) a SARS-COV-2 S glycoprotein of SEQ ID NO: 227 (referred to as BQ.1.1), (ii) a SARS-COV-2 S glycoprotein of SEQ ID NO: 274 (referred to as XBB.1.5), (iii) a first SARS-COV-2 S glycoprotein of SEQ ID NO: 227 and a second SARS-COV-2 S glycoprotein of SEQ ID NO: 274 (referred to as BQ.1.1+XBB.1.5), or (iv) a first SARS-CoV-2 S glycoprotein of SEQ ID NO: 222 and a second SARS-COV-2 S glycoprotein of SEQ ID NO: 274 (referred to as BA.5+XBB.1.5). The 1 month booster contained a saponin adjuvant and (i) and a SARS-COV-2 S glycoprotein of SEQ ID NO: 274 (XBB.1.5) or (ii) a SARS-COV-2 S glycoprotein of SEQ ID NO: 260 (XBB.1.16) The immunogenic compositions were administered according to the schedule in FIG. 17.

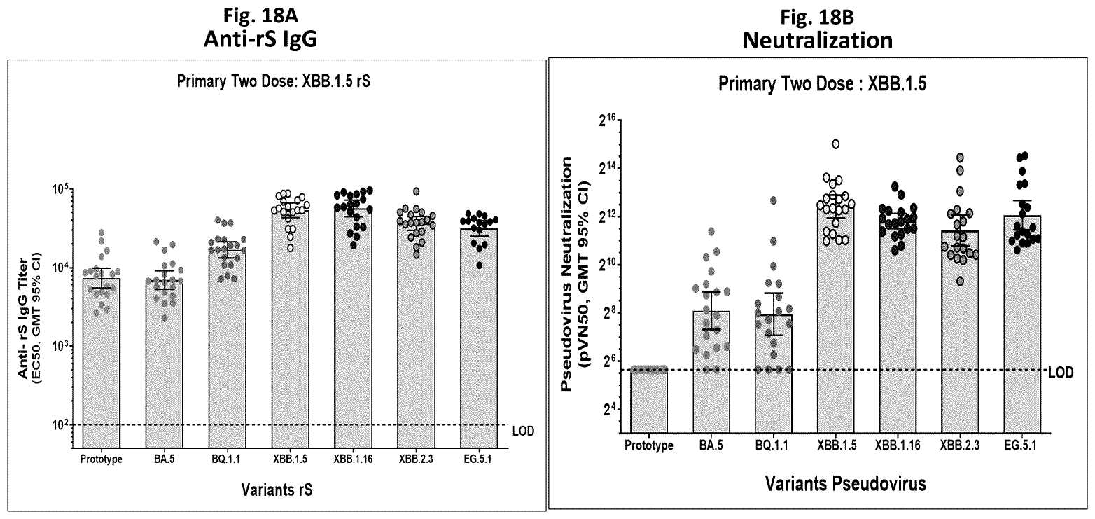

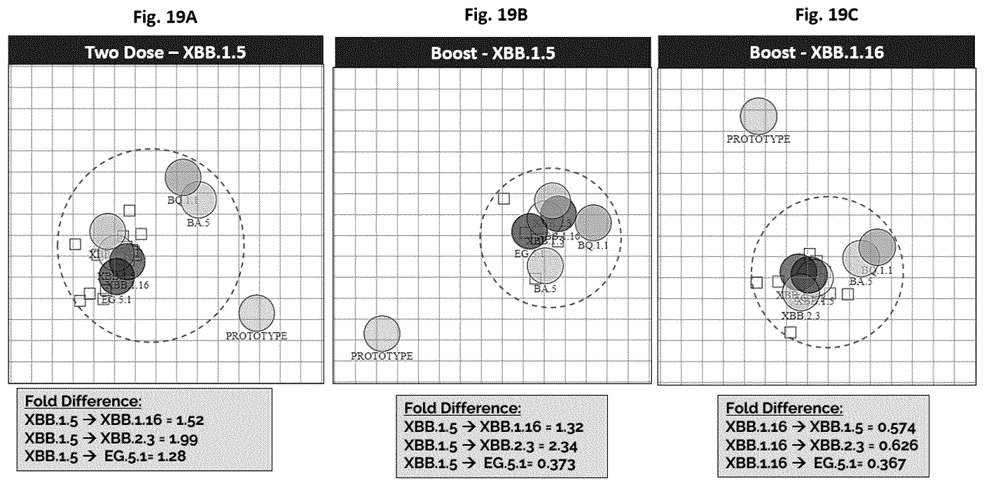

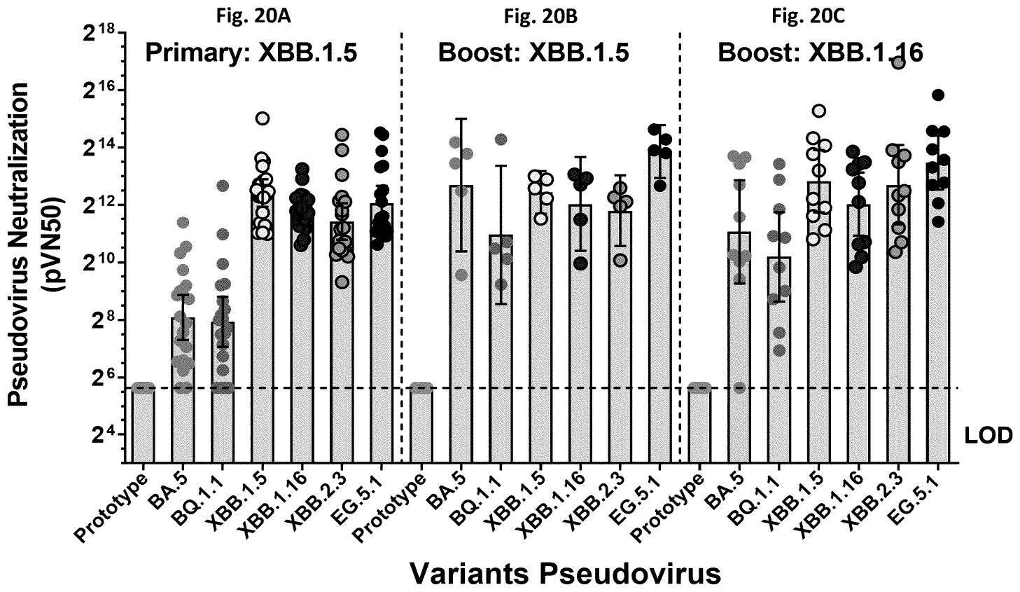

FIG. 18A shows the anti-S IgG antibodies in serum of mice after administration of two doses of an immunogenic composition comprising the SARS-COV 2 S glycoprotein of SEQ ID NO: 274. FIG. 18B shows the neutralizing antibodies present in the serum of mice after administration of two doses of an immunogenic composition comprising the SARS-COV 2 S glycoprotein of SEQ ID NO: 274. FIG. 19A shows the antigenic cartography of neutralizing responses induced by immunizing with two doses of an immunogenic composition comprising the SARS-COV 2 S glycoprotein of SEQ ID NO: 274. FIG. 19B shows the antigenic cartography of neutralizing responses induced by boosting with an immunogenic composition comprising the SARS-COV 2 S glycoprotein of SEQ ID NO: 274. FIG. 19C shows the antigenic cartography of neutralizing responses induced by boosting with an immunogenic composition comprising the SARS-COV 2 S glycoprotein of SEQ ID NO: 260. FIG. 20A shows pseudovirus neutralizing titers induced by administering a SARS-COV-2 S glycoprotein of SEQ ID NO: 274 in the primary series. FIG. 20B shows pseudovirus neutralizing titers induced by administering a SARS-COV-2 S glycoprotein of SEQ ID NO: 274 in the one month boost. FIG. 20C shows pseudovirus neutralizing titers induced by administering a SARS-COV-2 S glycoprotein of SEQ ID NO: 260 in the one month boost.

### Example 10

### Immunogenicity of Immunogenic Compositions in Rhesus Macaques

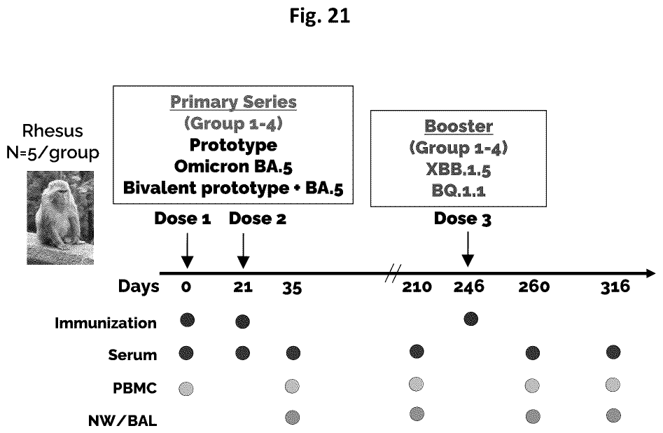

Immunogenic compositions comprising one or two SARS-COV-2 S glycoproteins and a saponin adjuvant were administered to Rhesus macaques (n=5). The saponin adjuvant contained two iscom particles, wherein: the first iscom particle comprises fraction A of Quillaja Saponaria Molina and not fraction C of Quillaja Saponaria Molina; and the second iscom particle comprises fraction C of Quillaja Saponaria Molina and not fraction A of Quillaja Saponaria Molina. Fraction A and Fraction C account for 85% and 15% by weight, respectively, of the sum of the weights of fraction A of Quillaja Saponaria Molina and fraction C of Quillaja Saponaria Molina in the adjuvant. The immunogenic compositions were administered according to the schedule in FIG. 21. The macaques were administered a first dose and second dose of a first immunogenic composition on day 0 and day 21 (referred to as the “primary series”). The macaques were administered a boost dose of a second immunogenic composition on day 246 (referred to as “booster”).

The first and second immunogenic composition each contained 5 μg SARS-COV-2 S glycoprotein and 50 μg saponin adjuvant. The first compositions contained a saponin adjuvant and (i) a SARS-COV-2 S glycoprotein of SEQ ID NO: 87 (referred to as Prototype), (ii) a SARS-COV-2 S glycoprotein of SEQ ID NO: 222 (referred to as BA.5), (iii) a SARS COV-2 S glycoprotein of SEQ ID NO: 87 and a SARS-COV-2 S glycoprotein of SEQ ID NO: 222 (referred to as “Prototype+BA.5”), or (iv) a SARS-COV-2 S glycoprotein of SEQ ID NO: 274 (referred to as XBB.1.5″). The second immunogenic compositions contained a saponin adjuvant and (i) a SARS-COV-2 S glycoprotein of SEQ ID NO: 274 (referred to as XBB.1.5″) or (ii)) a SARS-COV-2 S glycoprotein of SEQ ID NO: 227 (referred to as BQ.1.1).

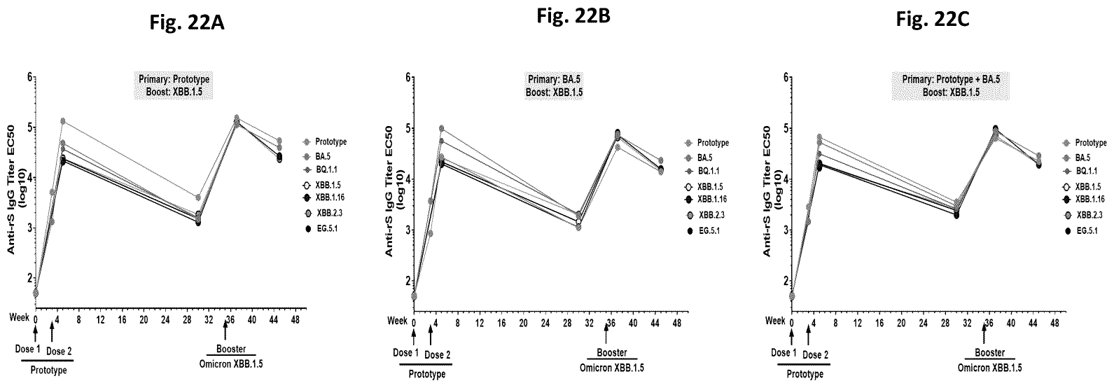

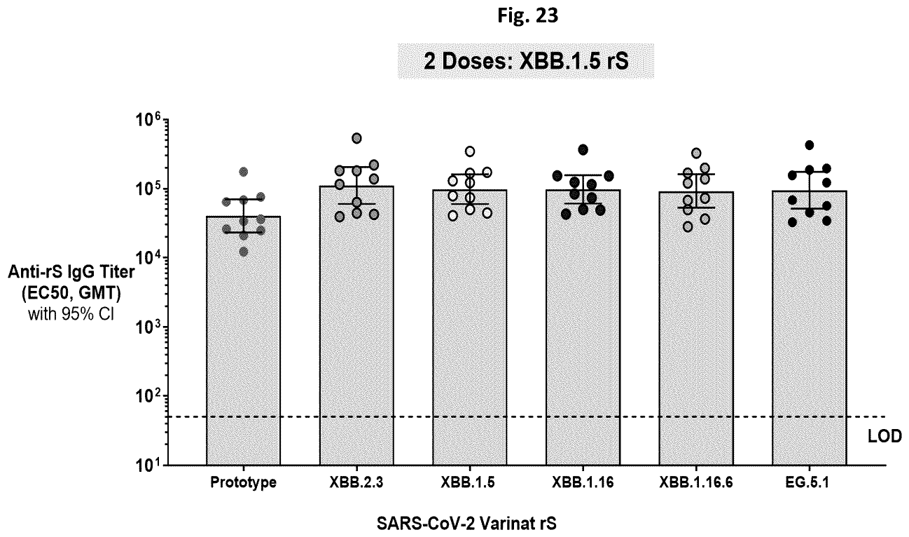

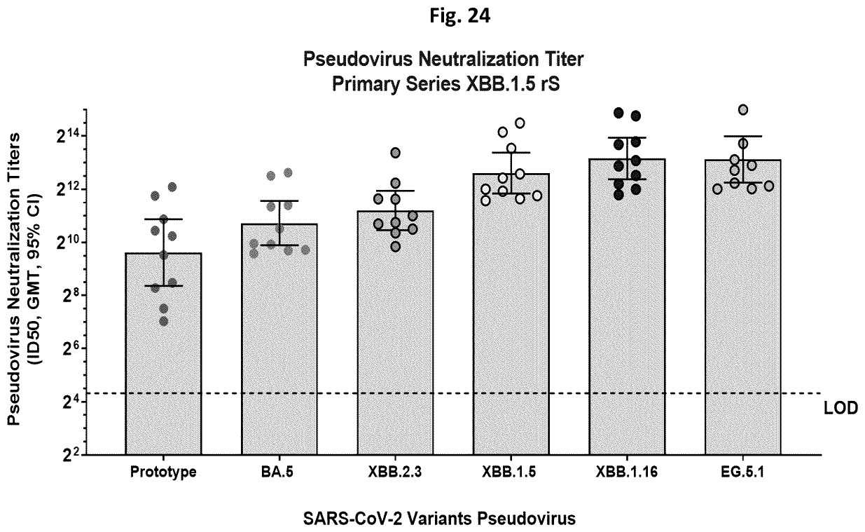

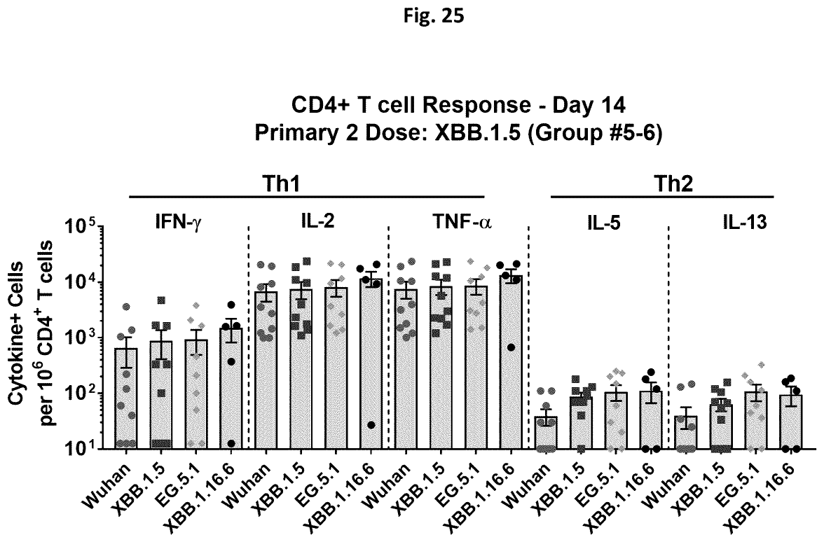

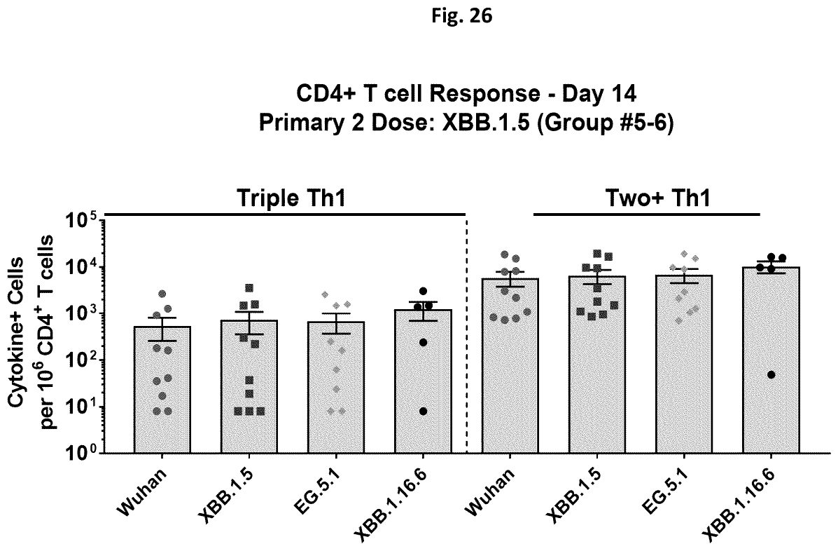

FIG. 22A shows the anti-S IgG antibodies in serum of macaques after administration of a first dose and a second dose containing the SARS-COV 2 S glycoprotein of SEQ ID NO: 87 and administration of a third dose containing a SARS-COV 2 S glycoprotein of SEQ ID NO: 274. FIG. 22B shows the anti-S IgG antibodies in serum of macaques after administration of a first dose and a second dose containing the SARS-COV 2 S glycoprotein of SEQ ID NO: 222 and administration of a third dose containing a SARS-COV 2 S glycoprotein of SEQ ID NO: 274. FIG. 22C shows the anti-S IgG antibodies in serum of macaques after administration of a first dose and a second dose containing the SARS-COV 2 S glycoprotein of SEQ ID NO: 87 and the SARS-COV-2 S glycoprotein of SEQ ID NO: 222 and administration of a third dose containing a SARS-COV 2 S glycoprotein of SEQ ID NO: 274. FIG. 23 shows the anti-S IgG antibodies in serum of macaques after administration of a first dose and a second dose containing the SARS-COV 2 S glycoprotein of SEQ ID NO: 274. FIG. 24 shows pseudovirus neutralizing titers induced by administering a SARS-COV-2 S glycoprotein of SEQ ID NO: 274 in the primary series. FIG. 25 shows the CD4+T cell response of macaques administered a first dose and a second dose containing the SARS-CoV 2 S glycoprotein of SEQ ID NO: 274. FIG. 26 shows the multifunctional antigen specific CD4+ T cell response of macaques administered a first dose and a second dose containing the SARS-COV 2 S glycoprotein of SEQ ID NO: 274.

### Example 11

### Immunogenicity of Immunogenic Compositions in Mice

Monovalent COVID-19 immunogenic compositions were prepared that contained one CoV S glycoprotein. Trivalent COVID-19 immunogenic compositions were also prepared which had three CoV S glycoproteins. The immunogenicity of the immunogenic compositions below was evaluated. Table C4 shows the identity of the CoV S glycoproteins in each composition.

TABLE C4

| | Amino acid sequence of CoV S |
|---|---|
| Composition | glycoprotein(s) in composition |
| 2 (also referred to as (“XBB.1.5”) | SEQ ID NO: 274 |
| 7 (also referred to as (“Prototype + | SEQ ID NO: 87 and |
| BA.5 + XBB.1.5”) | SEQ ID NO: 222 |
| | and SEQ ID NO: 274 |
| 8 (also referred to as “JN.1”) | SEQ ID NO: 329 |
| 9 (also referred to as “HV.1”) | SEQ ID NO: 334 |

Each monovalent COVID-19 immunogenic composition (Composition Nos. 2, 8-9) was prepared at 20 μg/mL CoV S rS+100 μg/mL Matrix-M to deliver 1 μg CoV S rS with 5 μg Matrix-M dose in 50 μL. Trivalent COVID-19 immunogenic compositions (Composition No. 7) were prepared at 20 μg/mL/each CoV S rS (60 μg/mL total CoV S rS)+100 μg/mL Matrix-M to deliver 1 μg CoV S rS/strain (3 μg rS total) with 5 μg Matrix-M dose in 50 μL.

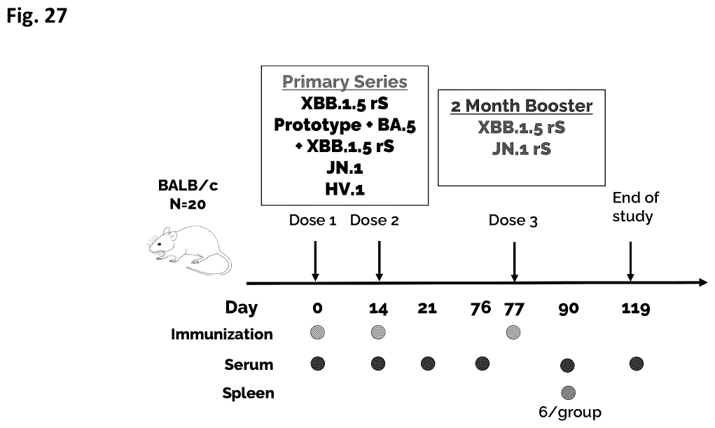

Groups of mice (n=20) were inoculated intramuscularly in the following regime, shown in FIG. 27:

| | | Primary | Booster (2 month) |
|---|---|---|---|
| | | Day 0 and 14 | Day 77 |
| Group | N | 1 μg rS + 5 μg Matrix-M | 1 μg rS + 5 μg Matrix-M |
| 1 | 20 | Composition 2 | Composition 2 |
| 2 | 20 | | Composition 8 |
| 4 | 20 | Composition 7 | Composition 2 |
| 5 | 20 | | Composition 8 |
| 8 | 20 | Composition 8 | Animals terminated after |
| 9 | 20 | Composition 9 | 2 dose primary series |

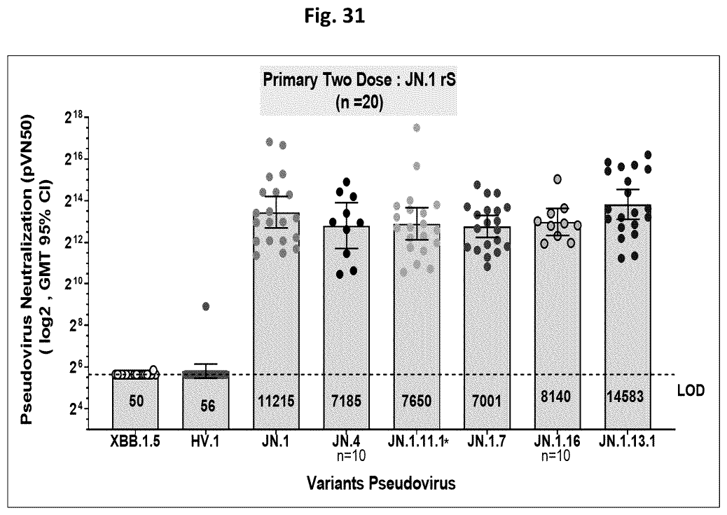

Administration of Composition 2 or Composition 7 or Composition 8 followed by a booster dose of Composition 8 elicited cross-reactive pseudovirus neutralizing antibodies against SARS-CoV-2 Variant Strains, XBB.1.5, HV.1, JN.1, JN. 4, JN.1.11.1, JN.1.7, JN.1.16, and JN.1.13.1 (FIGS. 31, 32D, 33C, 34D, 35B).

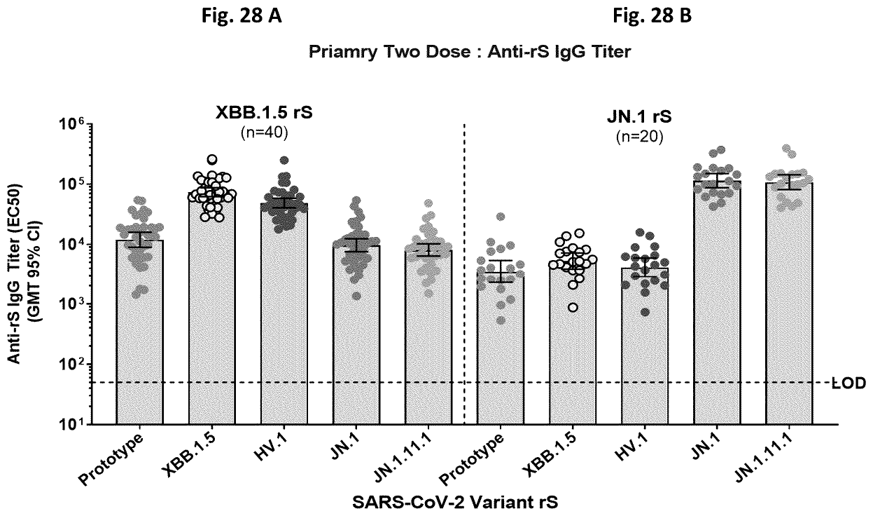

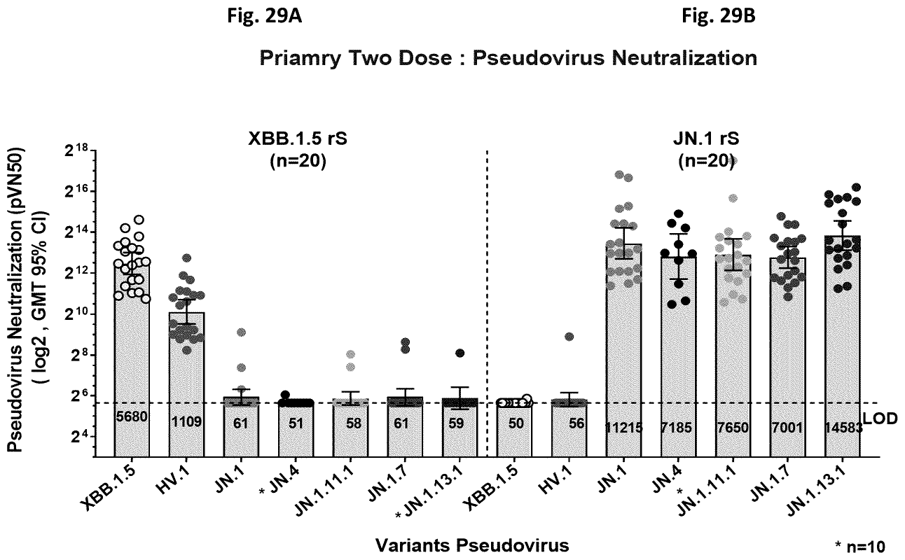

FIG. 28A shows the anti-rS IgG antibodies in serum of mice after administration of two doses of an immunogenic composition comprising the SARS-COV 2 S glycoprotein of SEQ ID NO: 274. FIG. 28B shows the neutralizing antibodies present in the serum of mice after administration of two doses of an immunogenic composition comprising the SARS-COV 2 S glycoprotein of SEQ ID NO: 329. FIG. 29A shows pseudovirus neutralizing titers induced by administering a SARS-COV-2 S glycoprotein of SEQ ID NO: 274 in the primary series. FIG. 29B shows pseudovirus neutralizing titers induced by administering a SARS-COV-2 S glycoprotein of SEQ ID NO: 329 in the primary series.

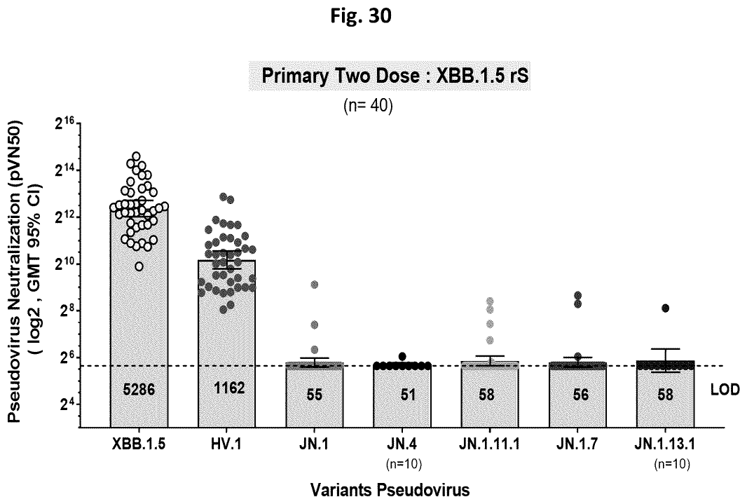

FIG. 30 shows pseudovirus neutralizing titers and antigenic distances induced by administering a SARS-COV-2 S glycoprotein of SEQ ID NO: 274 in the primary series against different SARS-COV-2 variant strains, with low titers for JN.1 and JN.1 subvariants. Specifically, pseudovirus neutralizing titers and antigenic distances after primary vaccination series with Composition 2 were JN.1 (pVNT50=55; AU=6.582), JN.4 (pVNT50=51; AU=6.684), JN.1.11.1 (pVNT50=58; AU=6.515), JN.1.7 (pVNT50=56; AU=6.568), and JN.1.13.1 (pVNT50=58; AU=6.500). FIG. 31 shows pseudovirus neutralizing titers and antigenic distances induced by administering a SARS-COV-2 S glycoprotein of SEQ ID NO: 329 in the primary series against different SARS-COV-2 variant strains, with high titers for JN.1 and JN.1 subvariants. Specifically, pseudovirus neutralizing titers and antigenic distances after primary vaccination series with Composition 8 were JN.1 (pVNT50=11,215; AU=−), JN.4 (pVNT50=7,185; AU=0.642), JN.1.11.1 (pVNT50=7,650; AU=0.552), JN.1.7 (pVNT50=7,001; AU=0.680), JN.1.13.1 (pVNT50=14,583; AU=−0.379), and JN.1.16 (pVNT50=8,140; AU=0.462) (FIG. 31) when compared to XBB.1.5 (pVNT50=50; AU=7.81) and HV.1 (pVNT50=56; AU=7.65) subvariant response which were low.

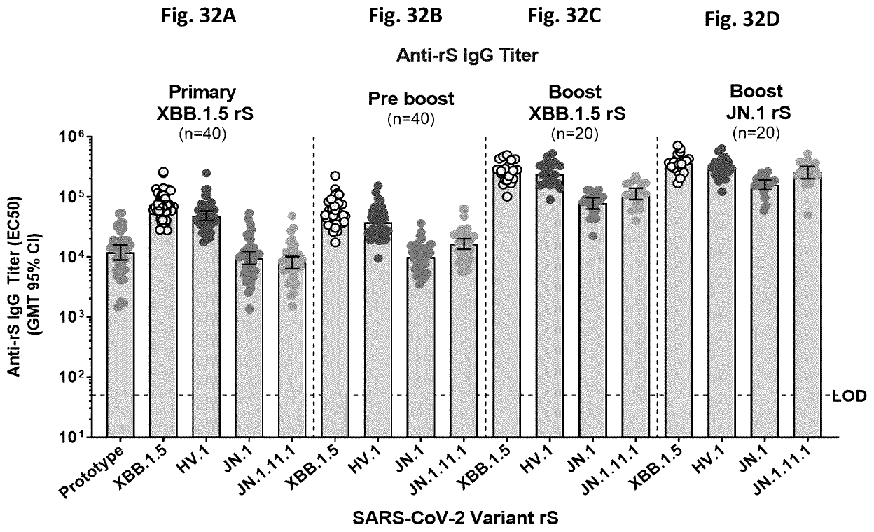

FIGS. 32A-32D show the anti-rS IgG antibodies in serum of mice after administration of Composition 2 in the primary series (FIG. 32A), compared to the anti-rS IgG antibodies in serum of mice prior to administration of a boost dose (FIG. 32B), against the anti-rS IgG antibodies in serum of mice induced after administration of a boost dose containing Composition 2 (FIG. 32C) or Composition 8 (FIG. 32D).

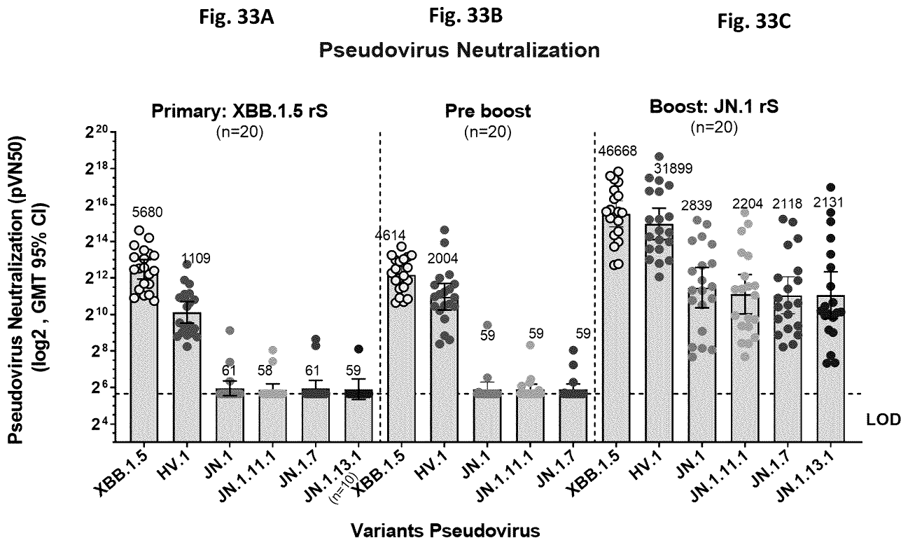

FIGS. 33A-33C show pseudovirus neutralizing titers and antigenic distances induced by administering a SARS-COV-2 S glycoprotein of SEQ ID NO: 274 in the primary series followed by a boost dose containing Composition 8 (FIG. 33C) of Example 11. Boosting with Composition 8 (FIG. 33C) elicited an improved immune response against pseudoviruses expressing S proteins from various SARS-COV-2 heterogeneous strains, as compared to pre-boosted mice (FIG. 33B) who received a primary dose of Composition 2 (FIG. 33A).

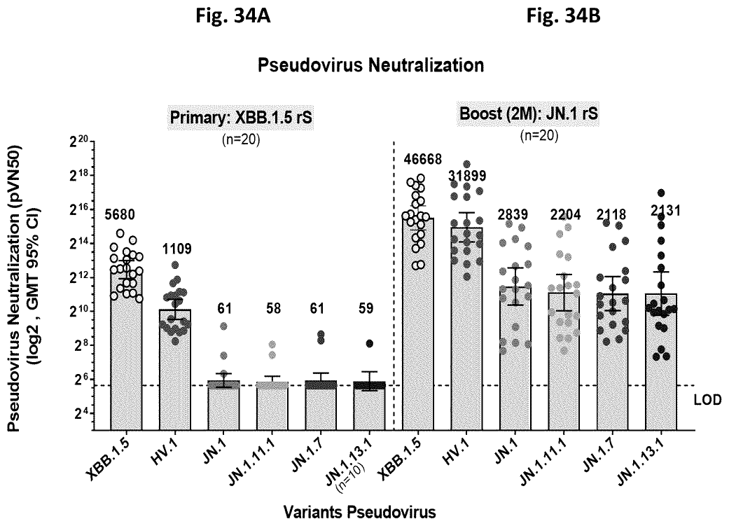

FIGS. 34A-34B show pseudovirus neutralizing titers and antigenic distances induced by administering Composition 2 (FIG. 34A) in the primary series followed by a boost dose after 2 months containing Composition 8 (FIG. 34B) of Example 11. Boosting with Composition 8 elicited an improved immune response against pseudoviruses expressing S proteins from various SARS-CoV-2 heterogeneous strains (FIG. 34B). Specifically, pseudovirus neutralizing titers and antigenic distances after primary vaccination series with Composition 2 followed by a booter dose at 2 months of Composition 8 were JN.1 (pVNT50=2839; AU=−), JN.1.11.1 (pVNT50=2204; AU=0.365), JN.1.7 (pVNT50=2118; AU=0.423), JN.1.13.1 (pVNT50=2131; AU=0.414), XBB.1.5 (pVNT50=46668; AU=−4.04), and HV.1 (pVNT50=31899; AU=−3.49) (FIG. 34B).

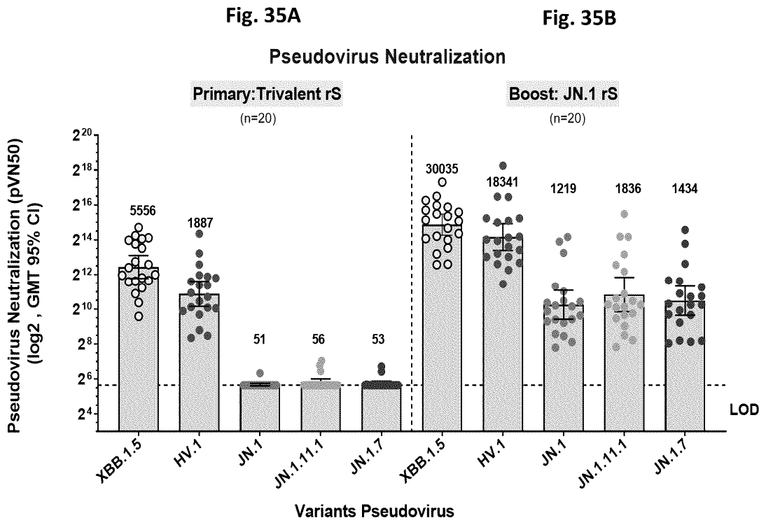

FIGS. 35A-35B show pseudovirus neutralizing titers and antigenic distances induced by administering Composition 7 (FIG. 35A) in the primary series followed by a boost dose after 2 months containing Composition 8 (FIG. 35B) of Example 11. Boosting with Composition 8 (FIG. 35B) elicited an improved immune response against pseudoviruses expressing S proteins from various SARS-COV-2 heterogeneous strains. Specifically, pseudovirus neutralizing titers and antigenic distances after primary vaccination series with Composition 7 followed by a booter dose at 2 months of Composition 8 were JN.1 (pVNT50=1219; AU=−), JN.1.11.1 (pVNT50=1836; AU=−0.591), JN.1.7 (pVNT50=1434; AU=−0.234), XBB.1.5 (pVNT50=30035; AU=−4.62), and HV.1 (pVNT50=18341; AU=−3.91).

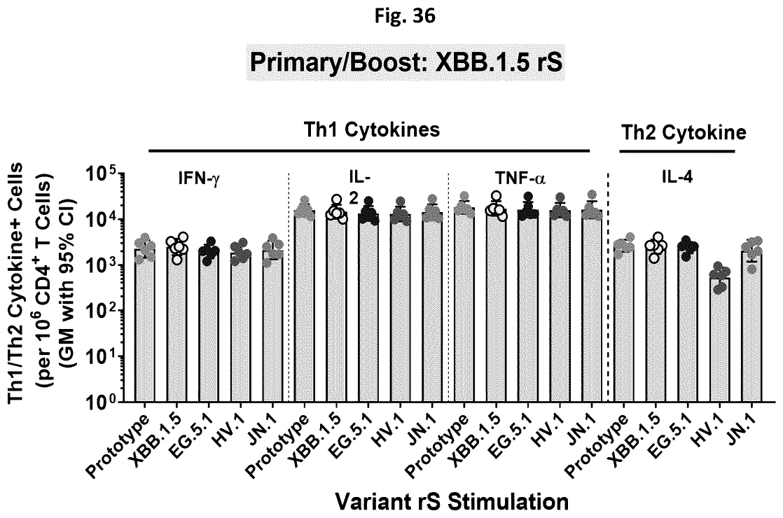

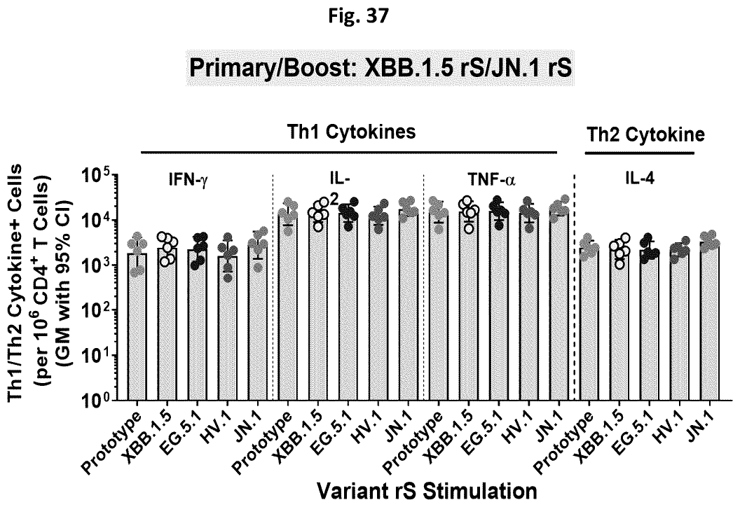

FIG. 36 shows the ratio of Th1/Th2 cytokines produced by CD4+ T cells in mice administered with doses of Composition 2 followed by a boost dose at 2 months with Composition 2. FIG. 37 shows the ratio of Th1/Th2 cytokines produced by CD4+ T cells in mice administered with doses of Composition 2 followed by a boost dose at 2 months with Composition 8. Th1 (IFN-Y, IL-2, and TNF-α) and Th2 (IL-4) cytokine-producing CD4+ T cells were measured in splenocytes isolated two month post booster dose with either Composition 2 or Composition 8. A robust CD4+ T cell response was recalled at comparable levels post boost upon boosting with Composition 8.

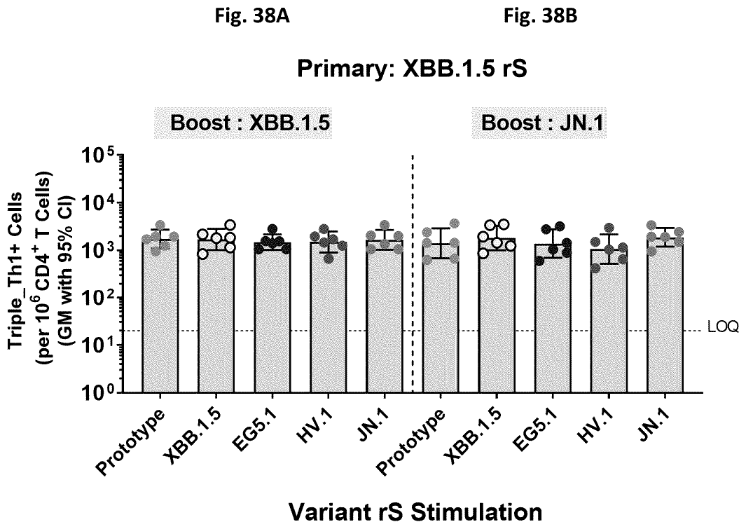

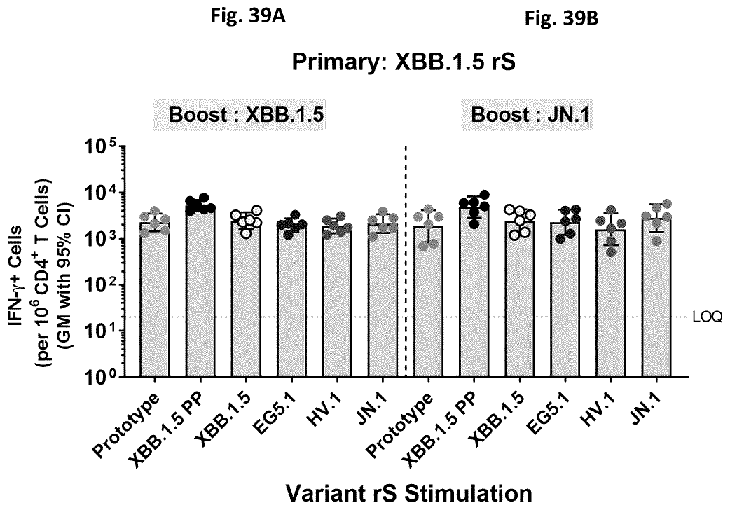

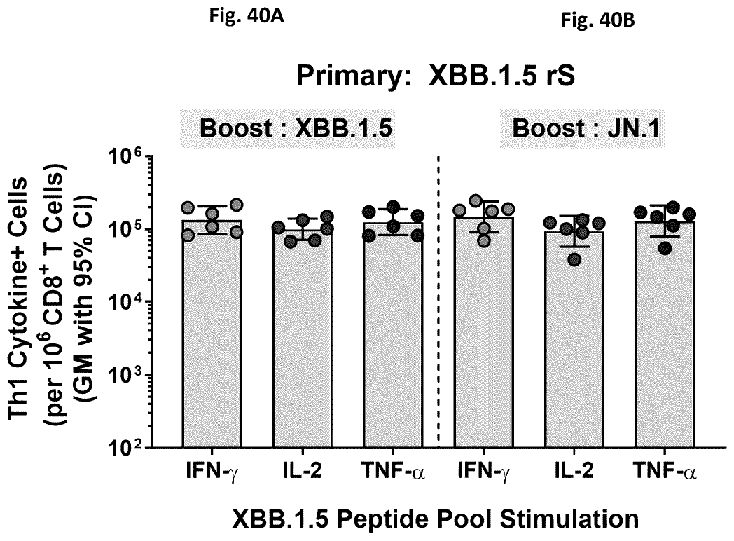

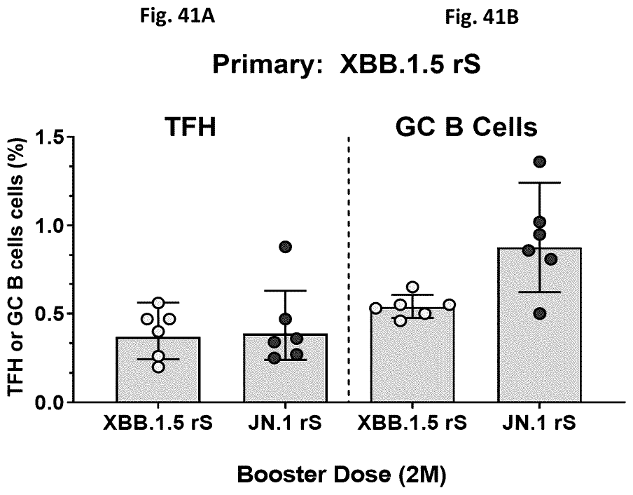

The effect of the immunogenic compositions on the T cell response are evaluated. Antigen-specific T cell responses are measured by ELISPOT™ enzyme linked immunosorbent assay and intracellular cytokine staining (ICCS) from spleens collected after the booster dose with Composition 2 or Composition 8 (study day 90). The frequency of IFN-γ+, TNF-α+, and IL-2+ cytokine-secreting CD4+ and CD8+ T cells is evaluated (FIGS. 38A-38B, 39A-39B, 40A-40B). Finally, the effect of immunization on germinal center formation is assessed by measuring the frequency of CD4+ T follicular helper (TFH) cells and germinal center (GC) B cells in spleens (FIGS. 41A-41B).

### Example 12

### Immunogenicity of Immunogenic Compositions in Rhesus Macaques

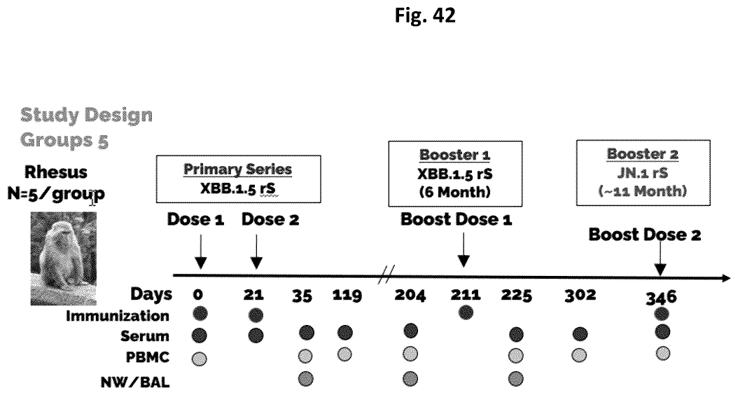

Immunogenic compositions comprising one or two SARS-COV-2 S glycoproteins and Matrix-M were administered to Rhesus macaques (n=5). The immunogenic compositions were administered according to the schedule in FIG. 42. The macaques were respectively administered a first dose and second dose of a first immunogenic composition on day 0 and day 21 (referred to as the “primary series”). The macaques were administered a boost dose of a second immunogenic composition on day 211 (referred to as “first booster”). The primed macaques were then administered a second boost dose of a third immunogenic composition on day 346 (referred to as “second booster”).

The immunogenic composition for intramuscular administration each contained 10 μg/mL SARS-COV-2 S glycoprotein and 100 μg/mL Matrix-M w to deliver 5 μg rS with 50 μg Matrix-M dose in 500 μL injection by intramuscular (IM) route. The immunogenic composition for IN administration each contained 50 μg/mL SARS-COV-2 S glycoprotein and 100 μg/mL Matrix-M to deliver 25 μg rS with 50 μg Matrix-M dose in 500 μL injection by IN route. The first and second compositions contained Matrix-M and a SARS-COV-2 S glycoprotein of SEQ ID NO: 274 (referred to as XBB.1.5). The third immunogenic composition contained Matrix-M and a SARS-CoV-2 S glycoprotein of SEQ ID NO: 329 (referred to as JN.1).

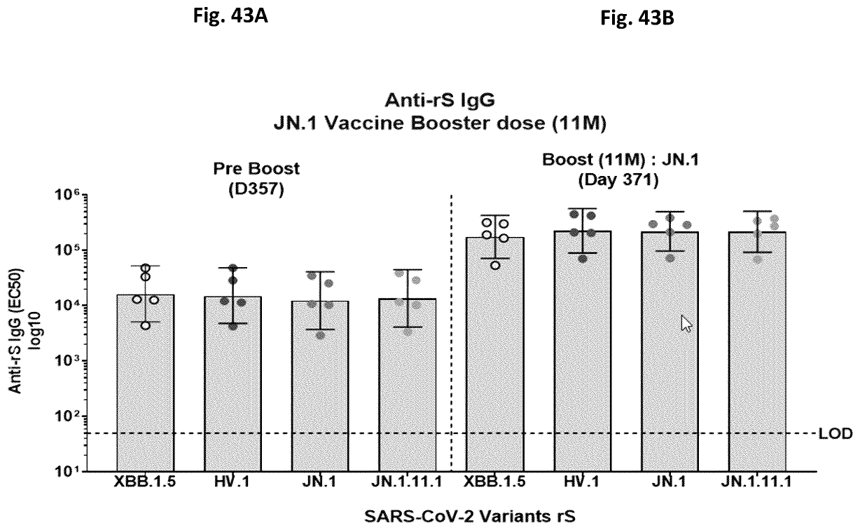

FIG. 43A shows the anti-rS responses in primed Rhesus macaques prior to administration of a second booster dose of an immunogenic composition comprising the SARS-COV 2 S glycoprotein of SEQ ID NO: 329. FIG. 43B shows the anti-rS responses in primed Rhesus macaques after administration of a second booster dose of an immunogenic composition comprising the SARS-COV 2 S glycoprotein of SEQ ID NO: 329.

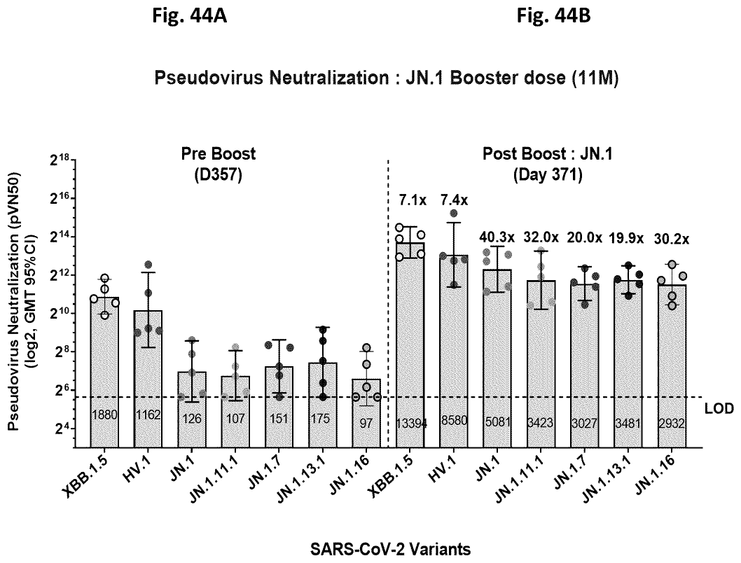

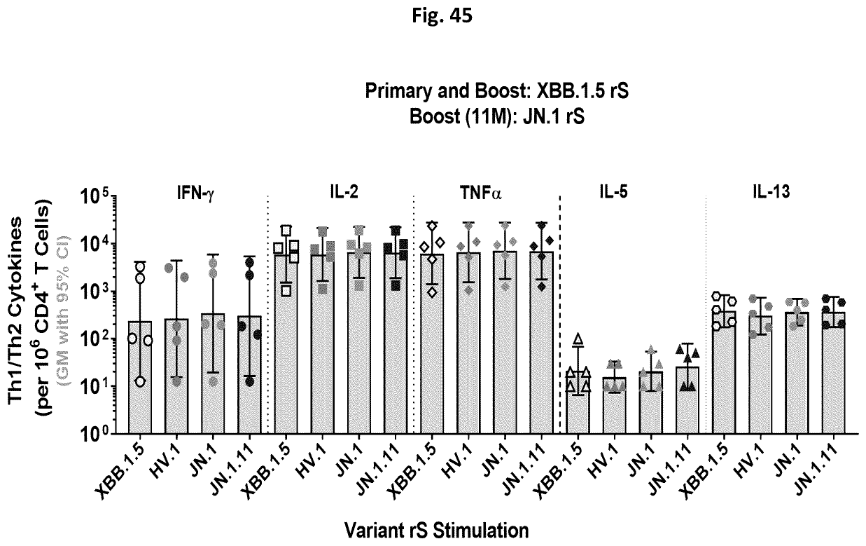

FIG. 44A shows pseudovirus neutralizing titers and antigenic distances in primed Rhesus macaques prior to administration of a second booster dose of an immunogenic composition comprising the SARS-COV 2 S glycoprotein of SEQ ID NO: 329. FIG. 44B shows pseudovirus neutralizing titers and antigenic distances in primed Rhesus macaques after administration of a second booster dose of an immunogenic composition comprising the SARS-COV 2 S glycoprotein of SEQ ID NO: 329. A second booster dose of an immunogenic composition comprising the SARS-COV 2 S glycoprotein of SEQ ID NO: 329 elicited pseudovirus neutralizing antibodies against different SARS-COV-2 variant strains.

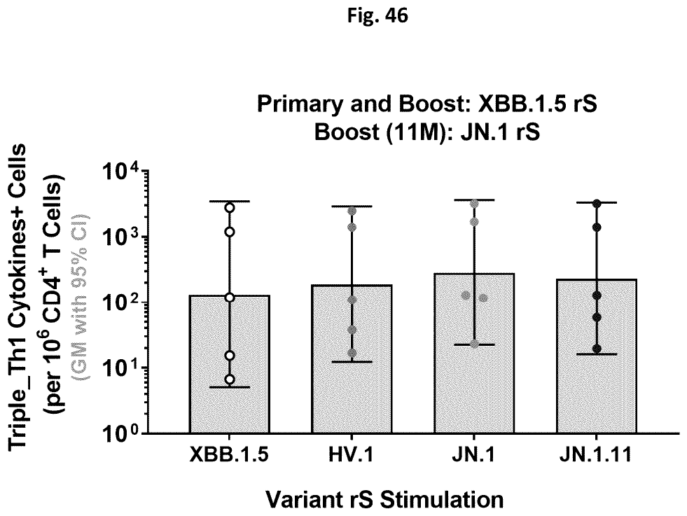

FIG. 45 shows the CD4+ T cell IFN-γ response in primed Rhesus macaques after administration of a second booster dose of an immunogenic composition comprising the SARS-CoV 2 S glycoprotein of SEQ ID NO: 329. A second booster dose of an immunogenic composition comprising the SARS-COV 2 S glycoprotein of SEQ ID NO: 329 elicited a Th1-biased cellular response and conserved T-cell epitopes recognized for all variants tested. FIG. 46 shows the multifunctional antigen specific CD4+ T cell response of primed Rhesus macaques after administration of a second booster dose of an immunogenic composition comprising the SARS-CoV 2 S glycoprotein of SEQ ID NO: 329.

### Enumerated Embodiments of the Disclosure

Specific enumerated embodiments <1> to <199> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.
1. An immunogenic composition comprising a first CoV S glycoprotein having an amino acid sequence with at least 80%, at least 85%, at least 90%, at least 91%, at least 92%, at least 93%, at least 94%, at least 95%, at least 96%, at least 97%, at least 98%, at least 99%, or 100% identity to any of SEQ ID NOS: 329-333.
2. The immunogenic composition of enumerated embodiment 1, wherein the first CoV S glycoprotein comprises an inactive furin cleavage site.
3. The immunogenic composition of any one of enumerated embodiments 1 and 2, comprising from 0.1 μg to 25 μg, 0.1 μg to 10 μg, 0.001 μg to 10 μg, 0.001 μg to 25 μg, or from 0.1 μg to 5 μg of the first CoV S glycoprotoein.
4. The immunogenic composition of enumerated embodiment 1, comprising a nanoparticle comprising the first CoV S glycoprotein and a non-ionic detergent core.
5. The immunogenic composition of enumerated embodiment 4, wherein the non-ionic detergent is selected from the group consisting of polysorbate-20 (PS20), polysorbate-40 (PS40), polysorbate-60 (PS60), polysorbate-65 (PS65), and polysorbate-80 (PS80).
6. The immunogenic composition of enumerated embodiment 1, further comprising an adjuvant and a pharmaceutically acceptable carrier.
7. The immunogenic composition of enumerated embodiment 8, wherein the adjuvant is a saponin adjuvant.
8. The immunogenic composition of enumerated embodiment 7, wherein the saponin adjuvant comprises at least two iscom particles, wherein:
   the first iscom particle comprises fraction A of Quillaja Saponaria Molina and not fraction C of Quillaja Saponaria Molina; and
   the second iscom particle comprises fraction C of Quillaja Saponaria Molina and not fraction A of Quillaja Saponaria Molina.
9. The immunogenic composition of enumerated embodiment 8, wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the saponin adjuvant.
10. The immunogenic composition of enumerated embodiment 8, wherein fraction A of Quillaja Saponaria Molina and fraction C of Quillaja Saponaria Molina account for about 92% by weight and about 8% by weight, respectively, of the sum of the weights of fraction A of Quillaja Saponaria Molina and fraction C of Quillaja Saponaria Molina in the saponin adjuvant.
11. The immunogenic composition of enumerated embodiment 8, wherein fraction A of Quillaja Saponaria Molina accounts for at least about 85% by weight, and fraction C of Quillaja Saponaria Molina accounts for the remainder, respectively, of the sum of the weights of fraction A of Quillaja Saponaria Molina and fraction C of Quillaja Saponaria Molina in the saponin adjuvant.
12. The immunogenic composition of enumerated embodiment 8, wherein fraction A of Quillaja Saponaria Molina accounts for 50-96% by weight and fraction C of Quillaja Saponaria Molina accounts for the remainder, respectively, of the sum of the weights of fraction A of Quillaja Saponaria Molina and fraction C of Quillaja Saponaria Molina in the saponin adjuvant.
13. The immunogenic composition of enumerated embodiment 6, comprising from about 25 μg to about 100 μg of adjuvant.
14. The immunogenic composition of enumerated embodiment 13, comprising about 50 μg or 75 μg of adjuvant.
15. The immunogenic composition of enumerated embodiment 1, wherein the first CoV S glycoprotein comprises one or more modifications compared to SEQ ID NO: 329 at amino acid 59, 346, 456, 475 572, and 1087, wherein the one or more modifications are numbered according to SEQ ID NO:1.
16. The immunogenic composition of enumerated embodiment 15, wherein:
(i) amino acid at 59 is phenylalanine or serine;
(ii) amino acid at 346 is arginine or threonine;
(iii) amino acid at 456 is phenylalanine or leucine;
(iv) amino acid at 475 is alanine or valine:
(v) amino acid at 572 is threonine or isoleucine; and
(vi) amino acid at 1087 is alanine or serine.
17. The immunogenic composition of any of enumerated embodiments 1-16, wherein the immunogenic composition is contained in a syringe.
18. A method of stimulating an immune response against SARS-COV-2 or a heterogeneous SARS-COV-2 strain thereof in a human comprising administering the immunogenic composition of any one of enumerated embodiments 1-16 to the human.
19. A method of boosting an immune response against SARS-COV-2 or a heterogeneous SARS-COV-2 strain thereof in a human comprising administering:
   a first immunogenic composition comprising (a) one or more first SARS-COV-2 S glycoproteins having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 329, and (b) a pharmaceutically acceptable buffer;
   wherein the immunogenic composition is administered after administration of another immunogenic composition intended to produce an immunogenic response against SARS-CoV-2 or a heterogeneous SARS-COV-2 strain thereof in the human.
20. The method of enumerated embodiment 19, comprising administering a first dose of the first immunogenic composition at least 21 days after administration of the other immunogenic composition.
21. The method of enumerated embodiment 20, comprising administering the first immunogenic composition at least 2, 3, 4, 5, 6, 7, 8, 9, 10, 11, 12, 13, 14, 15, 16, 17, 18, 19, 20, 21, 22, 23, or 24 months after administration of the other immunogenic composition.
22. The method of any one of enumerated embodiments 19-21, comprising administering the first immunogenic composition from about 1 month to about 18 months, from about 6 months to about 18 months, from about 9 months to about 18 months, from about 12 months to about 15 months, or from about 12 months to about 18 months after administration of the other immunogenic composition.
23. The method of any one of enumerated embodiments 19-22, wherein the other immunogenic composition comprises an mRNA encoding a SARS-Cov-2 Spike glycoprotein, a plasmid DNA encoding a SARS-Cov-2 Spike glycoprotein, a viral vector encoding a SARS-Cov-2 Spike glycoprotein, an inactivated SARS-COV-2 virus, or a protein subunit vaccine.
24. The method of enumerated embodiment 23, wherein the protein subunit vaccine of the other immunogenic composition comprises at least one or more second SARS-COV-2 S glycoproteins having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any of SEQ ID NOS: 87, 260, 222, 227, 274, and 284, and 329.
25. The method of enumerated embodiment 24, wherein the at least one or more second SARS-COV-2 S glycoproteins comprises at least two SARS-COV-2 S glycoproteins having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any of SEQ ID NOS: 87, 260, 222, 227, 274, and 284, and 329.
26. A method of broadening an immune response against SARS-COV-2 or a heterogeneous SARS-COV-2 strain thereof in a human comprising administering:
   a first immunogenic composition comprising (a) one or more first SARS-COV-2 S glycoproteins having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 329, and (b) a pharmaceutically acceptable buffer;
   wherein the immunogenic composition is administered after administration of another immunogenic composition intended to produce an immunogenic response against SARS-CoV-2 or a heterogeneous SARS-COV-2 strain thereof in the human, the other immunogenic composition being free of a SARS-COV-2 S glycoprotein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 329.
27. The method of enumerated embodiment 26, wherein the other immunogenic composition comprises one of an mRNA vaccine and a protein subunit vaccine.
28. The method of enumerated embodiment 27, wherein the other immunogenic composition comprises a second CoV S glycoprotein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NOS: 87, 260, 222, 227, 274, and 284.
29. A method of inducing an immune response against SARS-COV-2 or a heterogeneous SARS-COV-2 strain thereof in a human comprising administering:
   a first immunogenic composition comprising (a) one or more first SARS-COV-2 S glycoproteins having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 329, and (b) a pharmaceutically acceptable buffer
30. A method as recited in enumerated embodiment 29, further comprising administering a second immunogenic composition, comprising (a) one or more second SARS-COV-2 S glycoproteins having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any of SEQ ID NOS: 87, 260, 222, 227, 274, and 284, and 329.
31. The method of enumerated embodiment 30, comprising administering the second immunogenic composition at least 21 days after administration of the first immunogenic composition.
32. The method of enumerated embodiment 31, comprising administering the second immunogenic composition at least 2, 3, 4, 5, 6, 7, 8, 9, 10, 11, 12, 13, 14, 15, 16, 17, 18, 19, 20, 21, 22, 23, or 24 months after administration of the first immunogenic composition.
33. The method of any one of enumerated embodiments 30-32, comprising administering the second immunogenic composition from about 1 month to about 18 months, from about 6 months to about 18 months, from about 9 months to about 18 months, from about 12 months to about 15 months, or from about 12 months to about 18 months after administration of the first immunogenic composition.
34. An immunogenic composition comprising:
   (i) a first coronavirus spike (CoV S) glycoprotein;
   (ii) a second CoV S glycoprotein comprising from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein; and
   (iii) a third CoV S glycoprotein comprising from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein;
   wherein the second and third CoV S glycoproteins have different amino acid sequences.
35. The immunogenic composition of enumerated embodiment 34, wherein the first CoV S glycoprotein contains an inactive furin cleavage site and mutations of amino acids 973 and 974, wherein the amino acid sequence of the CoV S glycoprotein is numbered according to SEQ ID NO: 2.
36. The immunogenic composition of enumerated embodiment 34 or 35, comprising a fourth CoV S glycoprotein comprising from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein;
wherein the second, third, and fourth CoV S glycoproteins have different amino acid sequences.
37. The immunogenic composition of any one of enumerated embodiments 34-36, comprising a fifth CoV S glycoprotein comprising from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein;
   wherein the second, third, fourth, and fifth CoV S glycoproteins have different amino acid sequences.
38. The immunogenic composition of any one of enumerated embodiments 34-37, wherein one or more of the second, third, fourth, and fifth CoV S glycoproteins comprise modifications in the receptor binding domain (RBD).
39. The immunogenic composition of any one of enumerated embodiments 34-37, wherein one or more of the second, third, fourth, and fifth CoV S glycoproteins comprise one or more modifications in the S1 subunit.
40. The immunogenic composition of any one of enumerated embodiments 34-39, wherein one or more of the second, third, fourth, and fifth CoV S glycoproteins comprise one or more modifications in the S2 subunit.
41. The immunogenic composition of any one of enumerated embodiments 34-40, wherein one or more of the second, third, fourth, and fifth CoV S glycoproteins comprise one or more modifications in the N-terminal domain (NTD).
42. The immunogenic composition of any one of enumerated embodiments 34-41, wherein one or more of the second, third, fourth, and fifth CoV S glycoproteins comprise one or more modifications to any one of amino acids 630-710, any one of amino acids 640-700, any one of amino acids 650-690, or any one of amino acids 660-680, wherein the amino acids are numbered according to SEQ ID NO: 2.
43. The immunogenic composition of any one of enumerated embodiments 35-42, wherein the inactive furin cleavage site of the first CoV S glycoprotein comprises the amino acid sequence of QQAQ (SEQ ID NO: 7) and wherein amino acids 973 and 974 are proline.
44. The immunogenic composition of any one of enumerated embodiments 34-43, wherein one or more of the second, third, fourth, or fifth CoV S glycoprotein comprise an inactive furin cleavage site and mutations of amino acids 973 and 974, wherein the amino acid sequences of the second, third, fourth, and fifth CoV S glycoproteins are numbered according to SEQ ID NO: 2, optionally wherein, the inactive furin cleavage site comprises the amino acid sequence of QQAQ (SEQ ID NO: 7) and wherein amino acids 973 and 974 are proline.
45. The immunogenic composition of any one of enumerated embodiments 34-44, wherein the first CoV S glycoprotein comprises an amino acid sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 95%, at least 96%, at least 97%, at least 98%, at least 99% identical to any one of SEQ ID NOS: 86, 87, 88, 89, 105, 106, 109, 110, 112, 113, 115, 130, 132-134, 140-141, 143-144, 146, 147, 149, 151-153, 155-158, 174, 175, 186, 188, 190, 195, 217-228, 233-236, 243, and 245-292.
46. The immunogenic composition of any one of enumerated embodiments 34-45, wherein the first CoV S glycoprotein comprises an amino acid sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 95%, at least 96%, at least 97%, at least 98%, at least 99% identical to any one of SEQ ID NOS: 86, 87, 88, 89, 105, 106, 109, 110, 112, 113, 115, 130, 132-134, 140-141, 143-144, 146, 147, 149, 151-153, 155-158, 174, 175, 186, 188, 190, 195, 217-228, 233-236, 243, and 245-292.
47. The immunogenic composition of any one of enumerated embodiments 34-46, wherein the second CoV S glycoprotein comprises an amino acid sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 95%, at least 96%, at least 97%, at least 98%, at least 99% identical to any one of SEQ ID NOS: 86, 87, 88, 89, 105, 106, 109, 110, 112, 113, 115, 130, 132-134, 140-141, 143-144, 146, 147, 149, 151-153, 155-158, 174, 175, 186, 188, 190, 195, 217-228, 233-236, 243, and 245-292.
48. The immunogenic composition of any one of enumerated embodiments 34-47, wherein the third CoV S glycoprotein comprises an amino acid sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 95%, at least 96%, at least 97%, at least 98%, at least 99% identical to any one of SEQ ID NOS: 86, 87, 88, 89, 105, 106, 109, 110, 112, 113, 115, 130, 132-134, 140-141, 143-144, 146, 147, 149, 151-153, 155-158, 174, 175, 186, 188, 190, 195, 217-228, 233-236, 243, and 245-292.
49. The immunogenic composition of any one of enumerated embodiments 36-48, wherein the fourth CoV S glycoprotein comprises an amino acid sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 95%, at least 96%, at least 97%, at least 98%, at least 99% identical to any one of SEQ ID NOS: 86, 87, 88, 89, 105, 106, 109, 110, 112, 113, 115, 130, 132-134, 140-141, 143-144, 146, 147, 149, 151-153, 155-158, 174, 175, 186, 188, 190, 195, 217-228, 233-236, 243, and 245-292.
50. The immunogenic composition of any one of enumerated embodiments 37-49, wherein the fifth CoV S glycoprotein comprises an amino acid sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 95%, at least 96%, at least 97%, at least 98%, at least 99% identical to any one of SEQ ID NOS: 86, 87, 88, 89, 105, 106, 109, 110, 112, 113, 115, 130, 132-134, 140-141, 143-144, 146, 147, 149, 151-153, 155-158, 174, 175, 186, 188, 190, 195, 217-228, 233-236, 243, and 245-292.
51. The immunogenic composition of any one of enumerated embodiments 37-50, wherein each of the second, third, fourth, and fifth CoV S glycoproteins comprise one modification in the RBD compared to the first CoV S glycoprotein.
52. The immunogenic composition of enumerated embodiment 51, wherein the modification in the RBD is one or more of a P508S mutation, a K431T mutation, a F473S mutation, or a T465K mutation, wherein the amino acid sequence of each CoV S glycoprotein is numbered according to SEQ ID NO: 2.
53. The immunogenic composition of any one of enumerated embodiments 34-52, comprising from about 3 μg to about 7 μg of the first CoV S glycoprotein.
54. The immunogenic composition of any one of enumerated embodiments 34-53, comprising from about 1 ng to about 1 μg or from about 3 μg to about 7 μg of the second CoV S glycoprotein.
55. The immunogenic composition of any one of enumerated embodiments 34-54, comprising from about 1 ng to about 1 μg or from about 3 μg to about 7 μg of the third CoV S glycoprotein.
56. The immunogenic composition of any one of enumerated embodiments 37-53, comprising from about 1 ng to about 1 μg or from about 3 μg to about 7 μg of the fourth CoV S glycoprotein.
57. The immunogenic composition of any one of enumerated embodiments 37-53, comprising from about 1 ng to about 1 μg or from about 3 μg to about 7 μg of the fifth CoV S glycoprotein.
58. An immunogenic composition comprising:
   (i) a first nanoparticle comprising a first CoV S glycoprotein;
   (ii) a second nanoparticle comprising a second CoV S glycoprotein and a third CoV S glycoprotein, wherein each of the second CoV S glycoprotein and the third CoV S glycoprotein comprise from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein.
59. The immunogenic composition of enumerated embodiment 25, wherein the first CoV S glycoprotein contains an inactive furin cleavage site and mutations of amino acids 973 and 974, wherein the amino acid sequence of the CoV S glycoprotein is numbered according to SEQ ID NO: 2.
60. The immunogenic composition of enumerated embodiment 58 or 59, comprising a fourth CoV S glycoprotein comprising from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein;
   wherein the second, third, and fourth CoV S glycoproteins have different amino acid sequences.
61. The immunogenic composition of any one of enumerated embodiments 58-60, comprising a fifth CoV S glycoprotein comprising from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein;
   wherein the second, third, fourth, and fifth CoV S glycoproteins have different amino acid sequences.
62. The immunogenic composition of any one of enumerated embodiments 58-61, wherein one or more of the second, third, fourth, and fifth CoV S glycoproteins comprise modifications in the receptor binding domain (RBD).
63. The immunogenic composition of any one of enumerated embodiments 58-62, wherein one or more of the second, third, fourth, and fifth CoV S glycoproteins comprise one or more modifications in the S1 subunit.
64. The immunogenic composition of any one of enumerated embodiments 58-63, wherein one or more of the second, third, fourth, and fifth CoV S glycoproteins comprise one or more modifications in the S2 subunit.
65. The immunogenic composition of any one of enumerated embodiments 58-64, wherein one or more of the second, third, fourth, and fifth CoV S glycoproteins comprise one or more modifications in the N-terminal domain (NTD).
66. The immunogenic composition of any one of enumerated embodiments 58-65, wherein one or more of the second, third, fourth, and fifth CoV S glycoproteins comprise one or more modifications to any one of amino acids 630-710, any one of amino acids 640-700, any one of amino acids 650-690, or any one of amino acids 660-680, wherein the amino acids are numbered according to SEQ ID NO: 2.
67. The immunogenic composition of any one of enumerated embodiments 59-66, wherein the inactive furin cleavage site of the first CoV S glycoprotein comprises the amino acid sequence of QQAQ (SEQ ID NO: 7) and wherein amino acids 973 and 974 are proline.
68. The immunogenic composition of any one of enumerated embodiments 58-67, wherein one or more of the second, third, fourth, or fifth CoV S glycoprotein comprise an inactive furin cleavage site and mutations of amino acids 973 and 974, wherein the amino acid sequences of the second, third, fourth, and fifth CoV S glycoproteins are numbered according to SEQ ID NO: 2.
69. The immunogenic composition of enumerated embodiment 68, wherein the inactive furin cleavage site comprises the amino acid sequence of QQAQ (SEQ ID NO: 7) and wherein amino acids 973 and 974 are proline.
70. The immunogenic composition of any one of enumerated embodiments 58-69, wherein the first CoV S glycoprotein comprises an amino acid sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 95%, at least 96%, at least 97%, at least 98%, at least 99% identical to any one of SEQ ID NOS: 86, 87, 88, 89, 105, 106, 109, 110, 112, 113, 115, 130, 132-134, 140-141, 143-144, 146, 147, 149, 151-153, 155-158, 174, 175, 186, 188, 190, 195, 217-228, 233-236, 243, and 245-292.
71. The immunogenic composition of any one of enumerated embodiments 58-70, wherein the first CoV S glycoprotein comprises an amino acid sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 95%, at least 96%, at least 97%, at least 98%, at least 99% identical to any one of SEQ ID NOS: 245, 250, 255, and 260.
72. The immunogenic composition of any one of enumerated embodiments 58-71, wherein the second CoV S glycoprotein comprises an amino acid sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 95%, at least 96%, at least 97%, at least 98%, at least 99% identical to any one of SEQ ID NOS: 246, 251, 256, and 261.
73. The immunogenic composition of any one of enumerated embodiments 58-72, wherein the third CoV S glycoprotein comprises an amino acid sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 95%, at least 96%, at least 97%, at least 98%, at least 99% identical to any one of SEQ ID NOS: 247, 252, 257, and 262.
74. The immunogenic composition of any one of enumerated embodiments 60-73, wherein the fourth CoV S glycoprotein comprises an amino acid sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 95%, at least 96%, at least 97%, at least 98%, at least 99% identical to any one of SEQ ID NOS: 248, 253, 258, and 263.
75. The immunogenic composition of any one of enumerated embodiments 61-74, wherein the fifth CoV S glycoprotein comprises an amino acid sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 95%, at least 96%, at least 97%, at least 98%, at least 99% identical to any one of SEQ ID NOS: 249, 254, 259, and 264.
76. The immunogenic composition of any one of enumerated embodiments 61-75, wherein each of the second, third, fourth, and fifth CoV S glycoproteins comprise one modification in the RBD compared to the first CoV S glycoprotein.
77. The immunogenic composition of enumerated embodiment 76, wherein the modification is one or more of a P508S mutation, a K431T mutation, a F473S mutation, or a T465K mutation, wherein the amino acid sequence of each CoV S glycoprotein is numbered according to SEQ ID NO: 2
78. The immunogenic composition of any one of enumerated embodiments 58-77, comprising from about 3 μg to about 7 μg of the first CoV S glycoprotein.
79. The immunogenic composition of any one of enumerated embodiments 58-78, comprising from about 1 ng to about 1 μg or from about 3 μg to about 7 μg of the second CoV S glycoprotein.
80. The immunogenic composition of any one of enumerated embodiments 58-79, comprising from about 1 ng to about 1 μg or from about 3 μg to about 7 μg of the third CoV S glycoprotein.
81. The immunogenic composition of any one of enumerated embodiments 60-80, comprising from about 1 ng to about 1 μg or from about 3 μg to about 7 μg of the fourth CoV S glycoprotein.
82. The immunogenic composition of any one of enumerated embodiments 61-81, comprising from about 1 ng to about 1 μg or from about 3 μg to about 7 μg of the fifth CoV S glycoprotein.
83. The immunogenic composition of any one of enumerated embodiments 58-82, comprising a pharmaceutically acceptable buffer.
84. The immunogenic composition of any one of enumerated embodiments 58-83, comprising an adjuvant.
85. The immunogenic composition of enumerated embodiment 84, wherein the adjuvant is a saponin adjuvant.
86. The immunogenic composition of enumerated embodiment 85, wherein the saponin adjuvant comprises at least two iscom particles, wherein:
   the first iscom particle comprises fraction A of Quillaja Saponaria Molina and not fraction C of Quillaja Saponaria Molina; and
   the second iscom particle comprises fraction C of Quillaja Saponaria Molina and not fraction A of Quillaja Saponaria Molina.
87. The immunogenic composition of enumerated embodiment 86, wherein:
   fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the saponin adjuvant.
88. The immunogenic composition of enumerated embodiment 86, wherein fraction A of Quillaja Saponaria Molina and fraction C of Quillaja Saponaria Molina account for about 92% by weight and about 8% by weight, respectively, of the sum of the weights of fraction A of Quillaja Saponaria Molina and fraction C of Quillaja Saponaria Molina in the saponin adjuvant.
89. The immunogenic composition of enumerated embodiment 86, wherein fraction A of Quillaja Saponaria Molina accounts for at least about 85% by weight, and fraction C of Quillaja Saponaria Molina accounts for the remainder, respectively, of the sum of the weights of fraction A of Quillaja Saponaria Molina and fraction C of Quillaja Saponaria Molina in the saponin adjuvant.
90. The immunogenic composition of enumerated embodiment 86, wherein fraction A of Quillaja Saponaria Molina accounts for 50-96% by weight and fraction C of Quillaja Saponaria Molina accounts for the remainder, respectively, of the sum of the weights of fraction A of Quillaja Saponaria Molina and fraction C of Quillaja Saponaria Molina in the saponin adjuvant.
91. The immunogenic composition of any one of enumerated embodiments 84-90, comprising from about 25 μg to about 100 μg of adjuvant.
92. The immunogenic composition of any one of enumerated embodiments 84-90, comprising about 50 μg of adjuvant.
93. The immunogenic composition of any one of enumerated embodiments 1-92, comprising an mRNA encoding a SARS-Cov-2 Spike glycoprotein, a plasmid DNA encoding a SARS-Cov-2 Spike glycoprotein, a viral vector encoding a SARS-Cov-2 Spike glycoprotein, or an inactivated SARS-COV-2 virus.
94. The immunogenic composition of any one of enumerated embodiments 1-93, comprising at least one, at least two, at least three, or at least four hemagglutinin (HA) glycoproteins, wherein each HA glycoprotein is from a different influenza strain.
95. The immunogenic composition of any one of enumerated embodiments 1-94, comprising a respiratory syncytial virus (RSV) fusion (F) glycoprotein.
96. The immunogenic composition of any one of enumerated embodiments 1-95, wherein the total amount of CoV S glycoprotein in the composition is from about 3 μg to about 10 μg.
97. A pre-filled syringe comprising the immunogenic composition of any one of enumerated embodiments 34-96 and 125-196.
98. A method of stimulating an immune response against SARS-COV-2 or a heterogeneous SARS-COV-2 strain comprising administering the immunogenic composition of any one of enumerated embodiments 1-17 and 34-96 and 125-196 to a subject.
99. The method of enumerated embodiment 98, comprising administering 1, 2, 3, 4, 5, 6, 7, 8, 9, or 10 doses of the immunogenic composition.
100. The method of enumerated embodiment 98 or 99, comprising administering a first dose of the immunogenic composition and a second dose of the immunogenic composition about three weeks after the first dose.
101. The method of any one of enumerated embodiments 98-100, comprising administering a first dose of the immunogenic composition and a second dose of the immunogenic composition about 21 days after the first dose.
102. The method of any one of enumerated embodiments 98-101, comprising administering at least three doses of the immunogenic composition, wherein the third dose of the immunogenic composition is administered at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months after the first or second dose.
103. The method of any one of enumerated embodiments 98-102, comprising administering a second immunogenic composition that is different from the first immunogenic composition.
104. The method of enumerated embodiment 103, wherein the second immunogenic composition comprises an mRNA encoding a SARS-Cov-2 Spike glycoprotein, a plasmid DNA encoding a SARS-Cov-2 Spike glycoprotein, a viral vector encoding a SARS-Cov-2 Spike glycoprotein, or an inactivated SARS-COV-2 virus.
105. The method of enumerated embodiment 103, wherein the second immunogenic composition comprises at least one, at least two, at least three, or at least four hemagglutinin (HA) glycoproteins, wherein each HA glycoprotein is from a different influenza strain.
106. The method of enumerated embodiment 103, wherein the second immunogenic composition comprises an RSV F glycoprotein.
107. The method of enumerated embodiment 103, wherein the second immunogenic composition comprises a different CoV S glycoprotein than the first immunogenic composition.
108. The method of any one of enumerated embodiments 98-107, wherein the immunogenic composition is administered intramuscularly.
109. The method of any one of enumerated embodiments 98-108, comprising administering the immunogenic composition in a pre-filled syringe.
110. The method of any one of enumerated embodiments 98-109, wherein the method prevents COVID-19 with an efficacy from about 50% to about 99%, from about 50% to about 95%, from about 50% to about 90%, from about 50% to about 85%, from about 50% to about 80%, from about 60% to about 99%, from about 65% to about 95%, from about 65% to about 90%, from about 65% to about 85%, from about 69% to about 81%, from about 60% to about 95%, from about 60% to about 90%, from about 60% to about 85%, from about 60% to about 80%, from about 40% to about 99%, from about 40% to about 95%, from about 40% to about 90%, from about 40% to about 85%, from about 40% to about 80%, from about 40% to about 75%, from about 40% to about 70%, from about 40% to about 65%, from about 40% to about 55%, or from about 40% to about 50% for at least about 2 months, at least about 2.5 months, at least about 3 months, at least about 3.5 months, at least about 4 months, at least about 4.5 months, at least about 5 months, at least about 5.5 months, at least about 6 months, at least about 6.5 months, at least about 7 months, at least about 7.5 months, at least about 8 months, at least about 8.5 months, at least about 9 months, at least about 9.5 months, at least about 10 months, at least about 10.5 months, at least about 11 months, at least about 11.5 months, at least about 12 months, at least 13 months, at least 14 months, at least 15 months, at least 16 months, at least 17 months, at least 18 months, at least 19 months, at least 20 months, at least 21 months, at least 22 months, at least 23 months, or at least 24 months after administration of the immunogenic composition.
111. The method of any one of enumerated embodiments 98-110, wherein the method prevents COVID-19 with an efficacy from about 50% to about 99%, from about 50% to about 95%, from about 50% to about 90%, from about 50% to about 85%, from about 50% to about 80%, from about 60% to about 99%, from about 65% to about 95%, from about 65% to about 90%, from about 65% to about 85%, from about 69% to about 81%, from about 60% to about 95%, from about 60% to about 90%, from about 60% to about 85%, from about 60% to about 80%, from about 40% to about 99%, from about 40% to about 95%, from about 40% to about 90%, from about 40% to about 85%, from about 40% to about 80%, from about 40% to about 75%, from about 40% to about 70%, from about 40% to about 65%, from about 40% to about 55%, or from about 40% to about 50% for up to about 2 months, up to about 2.5 months, up to about 3 months, up to about 3.5 months, up to about 4 months, up to about 4.5 months, up to about 5 months, up to about 5.5 months, up to about 6 months, up to about 6.5 months, up to about 7 months, up to about 7.5 months, up to about 8 months, up to about 8.5 months, up to about 9 months, up to about 9.5 months, up to about 10 months, up to about 10.5 months, up to about 11 months, up to about 11.5 months, up to about 12 months, up to 13 months, up to 14 months, up to 15 months, up to 16 months, up to 17 months, up to 18 months, up to 19 months, up to 20 months, up to 21 months, up to 22 months, up to 23 months, or up to 24 months after administration of the immunogenic composition.
112. A method of stimulating an immune response against SARS-COV-2, a heterogeneous SARS-COV-2 strain, an influenza virus, or a combination thereof in a subject comprising administering the immunogenic composition of enumerated embodiment 94.
113. A method of stimulating an immune response against SARS-COV-2, a heterogeneous SARS-COV-2 strain, an influenza virus, respiratory syncytial virus (RSV) or a combination thereof in a subject comprising administering the immunogenic composition of enumerated embodiment 95.
114. A method of preparing an COV S nanoparticle, comprising the steps of:
   (i) binding a protein extract, wherein the protein extract comprises a first detergent and a first CoV S glycoprotein, to a protein purification column, wherein the column binds the CoV S glycoprotein;
   (ii) performing a detergent exchange by substantially replacing the first detergent with a second detergent; and
   (iii) eluting the bound CoV S glycoprotein from the column in the presence of the second detergent to provide the nanoparticle.
115. The method of enumerated embodiment 114, wherein the protein extract further comprises a second CoV S glycoprotein, wherein the second CoV S glycoprotein comprises from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein.
116. The method of enumerated embodiment 115, wherein the protein extract further comprises a third CoV S glycoprotein, wherein the third CoV S glycoprotein comprises from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein.
117. The method of enumerated embodiment 116, wherein the protein extract further comprises a fourth CoV S glycoprotein, wherein the fourth CoV S glycoprotein comprises from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein.
118. The method of enumerated embodiment 117, wherein the protein extract further comprises a fifth CoV S glycoprotein, wherein the fifth CoV S glycoprotein comprises from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein.
119. The method of any one of enumerated embodiments 114-118, wherein the protein extract is prepared by:
   (a) transforming a host cell with a nucleic acid encoding the first CoV S glycoprotein, wherein the glycoprotein comprises a transmembrane domain; and
   (b) culturing the host cell under conditions conducive to the production of the CoV S glycoprotein.
120. The method of enumerated embodiment 119, comprising transforming the host cell with a nucleic acid encoding a second CoV S glycoprotein, wherein the second CoV S glycoprotein comprises a transmembrane domain and wherein the second CoV S glycoprotein comprises from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein.
121. The method of enumerated embodiment 120, comprising transforming the host cell with a nucleic acid encoding a third CoV S glycoprotein, wherein the third CoV S glycoprotein comprises a transmembrane domain and wherein the third CoV S glycoprotein comprises from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein.
122. The method of enumerated embodiment 121, comprising transforming the host cell with a nucleic acid encoding a fourth CoV S glycoprotein, wherein the fourth CoV S glycoprotein comprises a transmembrane domain and wherein the fourth CoV S glycoprotein comprises from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein.
123. The method of enumerated embodiment 122, comprising transforming the host cell with a nucleic acid encoding a fifth CoV S glycoprotein, wherein the fifth CoV S glycoprotein comprises a transmembrane domain and wherein the fifth CoV S glycoprotein comprises from 1 to about 2, from 1 to about 3, from 1 to about 4, from 1 to about 5, from 2 to about 5, from 1 to about 6, from 1 to about 7, from 1 to about 8, from 1 to about 9, from 1 to about 10, from 1 to about 11, from 1 to about 12, from 1 to about 13, from 1 to about 14, from 1 to about 15, from 1 to about 16, from 1 to about 17, from 1 to about 18, from 1 to about 18, from 1 to about 19, or from 1 to about 20, from 1 to about 25, from 1 to about 30, from 1 to about 35, from 1 to about 40, from 1 to about 45, or from 1 to about 50 modifications compared to the first CoV S glycoprotein.
124. The method of any one of enumerated embodiments 119-123, wherein the host cell is an insect cell, optionally wherein the insect cell is a Sf9 or Sf22a cell.
125. An immunogenic composition comprising a first, second, third, fourth, and fifth CoV S glycoprotein.
126. The immunogenic composition of enumerated embodiment 125, wherein the first CoV S glycoprotein has an amino acid sequence with at least 90%, at least 91%, at least 92%, at least 93%, at least 94%, at least 95%, at least 96%, at least 97%, at least 98%, at least 99%, or 100% identity to any one of SEQ ID NOS: 245, 250, 255, 260, 265, 269, 273, 274, 277, and 284.
127. The immunogenic composition of enumerated embodiment 125 or 126, wherein the second, third, fourth, and fifth CoV S glycoprotein comprise from 1 to 60 modifications compared to the first CoV S glycoprotein.
128. The immunogenic composition of enumerated embodiment 127, wherein the second, third, fourth, and fifth CoV S glycoproteins comprise from 1 to 10 modifications compared to the first CoV S glycoprotein.
129. The immunogenic composition of enumerated embodiment 127 or 128, wherein the modifications occur at one or more of amino acids 180, 252, 253, 444, 478, 486, and 521, wherein the modifications are numbered according to a CoV S polypeptide having the amino acid sequence of SEQ ID NO: 1.
130. The immunogenic composition of any one of enumerated embodiments 125-129 comprising from 0.1 μg to 10 μg of each CoV S polypeptide.
131. The immunogenic composition of any one of enumerated embodiments 125-130, comprising from 0.1 μg to 10 μg of the first CoV S glycoprotein.
132. The immunogenic composition of any one of enumerated embodiments 125-131, comprising from 0.1 μg to 5 μg of the first CoV S glycoprotein.
133. The immunogenic composition of any one of enumerated embodiments 125-132, wherein the amount of the second CoV S glycoprotein in the composition is less than the amount of the first CoV S glycoprotein in the composition.
134. The immunogenic composition of any one of enumerated embodiments 125-133, wherein the amount of the third CoV S glycoprotein in the composition is less than the amount of the first CoV S glycoprotein in the composition.
135. The immunogenic composition of any one of enumerated embodiments 125-134, wherein the amount of the fourth CoV S glycoprotein in the composition is less than the amount of the first CoV S glycoprotein in the composition.
136. The immunogenic composition of any one of enumerated embodiments 125-135, wherein the amount of the fifth CoV S glycoprotein in the composition is less than the amount of the first CoV S glycoprotein in the composition.
137. The immunogenic composition of any one of enumerated embodiments 125-136, comprising an adjuvant.
138. The immunogenic composition of enumerated embodiment 137, wherein the adjuvant is a saponin adjuvant.
139. The immunogenic composition of enumerated embodiment 138, wherein the saponin adjuvant comprises at least two iscom particles, wherein:
   the first iscom particle comprises fraction A of Quillaja Saponaria Molina and not fraction C of Quillaja Saponaria Molina; and
   the second iscom particle comprises fraction C of Quillaja Saponaria Molina and not fraction A of Quillaja Saponaria Molina.
140. The immunogenic composition of enumerated embodiment 139, wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the saponin adjuvant.
141. The immunogenic composition of enumerated embodiment 139, wherein fraction A of Quillaja Saponaria Molina and fraction C of Quillaja Saponaria Molina account for about 92% by weight and about 8% by weight, respectively, of the sum of the weights of fraction A of Quillaja Saponaria Molina and fraction C of Quillaja Saponaria Molina in the saponin adjuvant.
142. The immunogenic composition of enumerated embodiment 139, wherein fraction A of Quillaja Saponaria Molina accounts for at least about 85% by weight, and fraction C of Quillaja Saponaria Molina accounts for the remainder, respectively, of the sum of the weights of fraction A of Quillaja Saponaria Molina and fraction C of Quillaja Saponaria Molina in the saponin adjuvant.
143. The immunogenic composition of enumerated embodiment 139, wherein fraction A of Quillaja Saponaria Molina accounts for 50-96% by weight and fraction C of Quillaja Saponaria Molina accounts for the remainder, respectively, of the sum of the weights of fraction A of Quillaja Saponaria Molina and fraction C of Quillaja Saponaria Molina in the saponin adjuvant.
144. The immunogenic composition of any one of enumerated embodiments 137-143, comprising from about 25 μg to about 100 μg of adjuvant.
145. The immunogenic composition of any one of enumerated embodiments 137-144, comprising about 50 μg of adjuvant.
146. The immunogenic composition of any one of enumerated embodiments 139-1145, wherein the second, third, fourth, or fifth CoV S glycoprotein comprises one or more modifications compared to the first CoV S glycoprotein at amino acid 180, 252, 253, 444, 478, 486, or 521, wherein the one or more modifications are numbered according to a CoV S glycoprotein of SEQ ID NO: 1.
147. The immunogenic composition of enumerated embodiment 146, wherein:
(i) amino acid 180 is glutamate or valine;
(ii) amino acid 252 is glycine or valine;
(iii) amino acid 253 is aspartate or glycine;
(iv) amino acid 444 is lysine or threonine;
(v) amino acid 478 is threonine, arginine, or lysine;
(vi) amino acid 486 is phenylalanine, proline, or serine; and
(vii) amino acid 521 is proline or serine.
148. An immunogenic composition comprising at least two CoV S glycoproteins.
149. The immunogenic composition of enumerated embodiment 148, wherein one CoV S glycoprotein has an amino acid sequence with at least 90%, at least 91%, at least 92%, at least 93%, at least 94%, at least 95%, at least 96%, at least 97%, at least 98%, at least 99%, or 100% identity to SEQ ID NO: 87.
150. The immunogenic composition of enumerated embodiment 148 or 149, wherein one CoV S glycoprotein has an amino acid sequence with at least 90%, at least 91%, at least 92%, at least 93%, at least 94%, at least 95%, at least 96%, at least 97%, at least 98%, at least 99%, or 100% identity to any one of SEQ ID NOS: 260, 274, and 284.
151. The immunogenic composition of any one of enumerated embodiments 148-150, wherein one CoV S glycoprotein comprises an amino acid sequence with at least 90%, at least 91%, at least 92%, at least 93%, at least 94%, at least 95%, at least 96%, at least 97%, at least 98%, at least 99%, or 100% identity to any one of SEQ ID NOS: 245, 250, 255, 260, 265, 269, 273, 274, 277, and 284.
152. The immunogenic composition of any one of enumerated embodiments 148-151, wherein the immunogenic composition comprises two CoV S glycoproteins.
153. The immunogenic composition of any one of enumerated embodiments 148-151, wherein the immunogenic composition comprises three CoV S glycoproteins.
154. The immunogenic composition of any one of enumerated embodiments 148-151, wherein the immunogenic composition comprises four CoV S glycoproteins.
155. The immunogenic composition of any one of enumerated embodiments 148-151, wherein the immunogenic composition comprises five CoV S glycoproteins.
156. The immunogenic composition of any one of enumerated embodiments 148-155, wherein the second CoV S glycoprotein comprises from 1 to 60 modifications compared to the first CoV S glycoprotein.
157. The immunogenic composition of any one of enumerated embodiments 153-156, wherein the third CoV S glycoprotein comprises from 1 to 60 modifications compared to the first CoV S glycoprotein.
158. The immunogenic composition of any one of enumerated embodiments 154-157, wherein the fourth CoV S glycoprotein comprises from 1 to 60 modifications compared to the first CoV S glycoprotein.
159. The immunogenic composition of any one of enumerated embodiments 155-158, wherein the fifth CoV S glycoprotein comprises from 1 to 60 modifications compared to the first CoV S glycoprotein.
160. The immunogenic composition of any one of enumerated embodiments 148-155, wherein the second CoV S glycoprotein comprises from 1 to 10 modifications compared to the first CoV S glycoprotein.
161. The immunogenic composition of any one of enumerated embodiments 153-156, wherein the third CoV S glycoprotein comprises from 1 to 10 modifications compared to the first CoV S glycoprotein.
162. The immunogenic composition of any one of enumerated embodiments 154-157, wherein the fourth CoV S glycoprotein comprises from 1 to 10 modifications compared to the first CoV S glycoprotein.
163. The immunogenic composition of any one of enumerated embodiments 155-158, wherein the fifth CoV S glycoprotein comprises from 1 to 10 modifications compared to the first CoV S glycoprotein.
164. The immunogenic composition of any one of enumerated embodiments 148-163, wherein at least one of the CoV S glycoproteins comprises an inactive furin cleavage site.
165. The immunogenic composition of enumerated embodiment 164, wherein the inactive furin cleavage site comprises the amino acid sequence of QQAQ (SEQ ID NO: 7).
166. The immunogenic composition of any one of enumerated embodiments 148-165, wherein at least one of the CoV S glycoproteins comprises mutation of amino acids 986 and 987 to proline, wherein the CoV S glycoprotein sequence is numbered according to SEQ ID NO: 1.
167. The immunogenic composition of any one of enumerated embodiments 148-166, wherein all of the CoV S glycoproteins in the composition comprise an inactive furin cleavage site.
168. The immunogenic composition of enumerated embodiment 167, wherein the inactive furin cleavage site comprises the amino acid sequence of QQAQ (SEQ ID NO: 7).
169. The immunogenic composition of any one of enumerated embodiments 148-168, wherein all of the CoV S glycoproteins comprise mutation of amino acids 986 and 987 to proline, wherein the CoV S glycoprotein sequence is numbered according to SEQ ID NO: 1.
170. The immunogenic composition of any one of enumerated embodiments 148-169 comprising from 0.1 μg to 25 μg, 0.1 μg to 10 μg, 0.001 μg to 10 μg, 0.001 μg to 25 μg, or from 0.1 μg to 5 μg of each CoV S polypeptide.
171. The immunogenic composition of any one of enumerated embodiments 148-170, wherein a first CoV S glycoprotein is present in the composition in an amount from about 1 μg to about 10 μg, and the other CoV S glycoproteins are each present in an amount that is less than ½, less than ⅓, less than ¼, less than ⅕, less than ⅙, less than 1/7, less than ⅛, less than 1/9, less than 1/10, less than 1/25, less than 1/50, less than 1/75, or less than 1/100 of the first CoV S glycoprotein.
172. The immunogenic composition of any one of enumerated embodiments 148-171, wherein the amount of the second CoV S glycoprotein in the composition is less than the amount of the first CoV S glycoprotein in the composition.
173. The immunogenic composition of any one of enumerated embodiments 153-172, wherein the amount of the third CoV S glycoprotein in the composition is less than the amount of the first CoV S glycoprotein in the composition.
174. The immunogenic composition of any one of enumerated embodiments 154-173, wherein the amount of the fourth CoV S glycoprotein in the composition is less than the amount of the first CoV S glycoprotein in the composition.
175. The immunogenic composition of any one of enumerated embodiments 155-174, wherein the amount of the fifth CoV S glycoprotein in the composition is less than the amount of the first CoV S glycoprotein in the composition.
176. The immunogenic composition of any one of enumerated embodiments 148-175, comprising an adjuvant.
177. The immunogenic composition of enumerated embodiment 176, wherein the adjuvant is a saponin adjuvant.
178. The immunogenic composition of enumerated embodiment 177, wherein the saponin adjuvant comprises at least two iscom particles, wherein:
   the first iscom particle comprises fraction A of Quillaja Saponaria Molina and not fraction C of Quillaja Saponaria Molina; and
   the second iscom particle comprises fraction C of Quillaja Saponaria Molina and not fraction A of Quillaja Saponaria Molina.
179. The immunogenic composition of enumerated embodiment 178, wherein fraction A of Quillaja Saponaria Molina and fraction C of Quillaja Saponaria Molina account for about 85% by weight and about 15% by weight, respectively, of the sum of weights of fraction A of Quillaja Saponaria Molina and fraction C of Quillaja Saponaria Molina in the saponin adjuvant.
180. The immunogenic composition of enumerated embodiment 178, wherein fraction A of Quillaja Saponaria Molina and fraction C of Quillaja Saponaria Molina account for about 92% by weight and about 8% by weight, respectively, of the sum of the weights of fraction A of Quillaja Saponaria Molina and fraction C of Quillaja Saponaria Molina in the saponin adjuvant.
181. The immunogenic composition of enumerated embodiment 178, wherein fraction A of Quillaja Saponaria Molina accounts for at least about 85% by weight, and fraction C of Quillaja Saponaria Molina accounts for the remainder, respectively, of the sum of the weights of fraction A of Quillaja Saponaria Molina and fraction C of Quillaja Saponaria Molina in the saponin adjuvant.
182. The immunogenic composition of enumerated embodiment 178, wherein fraction A of Quillaja Saponaria Molina accounts for 50-96% by weight and fraction C of Quillaja Saponaria Molina accounts for the remainder, respectively, of the sum of the weights of fraction A of Quillaja Saponaria Molina and fraction C of Quillaja Saponaria Molina in the saponin adjuvant.
183. The immunogenic composition of any one of enumerated embodiments 148-182, comprising from about 25 μg to about 100 μg of adjuvant.
184. The immunogenic composition of any one of enumerated embodiments 148-183, comprising about 50 μg or 75 μg of adjuvant.
185. The immunogenic composition of any one of enumerated embodiments 148-184, wherein one or more of the second, third, fourth, or fifth CoV S glycoprotein comprises one or more modifications compared to the first CoV S glycoprotein at amino acid 180, 252, 253, 444, 478, 486, or 521, wherein the one or more modifications are numbered according to a CoV S glycoprotein of SEQ ID NO: 1.
186. The immunogenic composition of enumerated embodiment 185, wherein:
   (i) amino acid 180 is glutamate or valine;
   (ii) amino acid 252 is glycine or valine;
   (iii) amino acid 253 is aspartate or glycine;
   (iv) amino acid 444 is lysine or threonine;
   (v) amino acid 478 is threonine, arginine, or lysine;
   (vi) amino acid 486 is phenylalanine, proline, or serine; and
   (vii) amino acid 521 is proline or serine.
187. An immunogenic composition comprising:
   (i) one or more non-naturally occurring SARS-COV-2 S glycoproteins having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NOS: 87, 260, 222, 227, 274, and 284; and
   (ii) a pharmaceutically acceptable buffer.
188. The immunogenic composition of enumerated embodiment 187, comprising: a non-naturally occurring SARS-COV-2 S glycoprotein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 274.
189. The immunogenic composition of any one of enumerated embodiments 187-188, comprising from about 1 μg to about 25 μg of SARS-COV-2 S glycoprotein.
190. The immunogenic composition of any one of enumerated embodiments 187-189, comprising a saponin adjuvant.
191. The immunogenic composition of any one of enumerated embodiments 187-190, comprising from about 25 to about 100 μg of saponin adjuvant.
192. The immunogenic composition of any one of enumerated embodiments 187-190, comprising from about 25 to about 50 μg of saponin adjuvant.
193. The immunogenic composition of any one of enumerated embodiments 187-192, comprising a nanoparticle comprising the one or more SARS-COV-2 S glycoproteins and a non-ionic detergent core.
194. The immunogenic composition of enumerated embodiment 193, wherein the non-ionic detergent is selected from the group consisting of polysorbate-20 (PS20), polysorbate-40 (PS40), polysorbate-60 (PS60), polysorbate-65 (PS65), and polysorbate-80 (PS80).
195. The immunogenic composition of any one of enumerated embodiments 187-194, wherein the saponin adjuvant comprises:
   (i) a first iscom particle comprising fraction A of Quillaja Saponaria Molina and not fraction C of Quillaja Saponaria Molina; and
   (ii) a second iscom particle comprising fraction C of Quillaja Saponaria Molina and not fraction A of Quillaja Saponaria Molina.
196. The immunogenic composition of enumerated embodiment 195, wherein fraction A of Quillaja Saponaria Molina and fraction C of Quillaja Saponaria Molina account for about 85% by weight and about 15% by weight, respectively, of the sum of the weights of fraction A of Quillaja Saponaria Molina and fraction C of Quillaja Saponaria Molina in the saponin adjuvant.
197. A method of stimulating an immune response against SARS-COV-2 or a heterogeneous SARS-COV-2 strain thereof in a human comprising administering an immunogenic composition of any one of enumerated embodiments 187-196.
198. The immunogenic composition of enumerated embodiment 1, wherein the first CoV S glycoprotein comprises an inactive furin cleavage site.
199. The immunogenic composition of enumerated embodiment 198, wherein the inactive furin cleavage site comprises the amino acid sequence of QQAQ (SEQ ID NO: 7).

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world. The following patent documents are incorporated by reference herein in their entireties for all purposes: International Publication No. 2021/154812; International Publication No. 2022/203963; International Publication No. 2022/235663; International Publication No. 2004/004762; International Publication No. 2019/183063; International Publication No. 2017/041100; and PCT/US2022/080700.

What is claimed is:

1. An immunogenic composition comprising a first CoV S glycoprotein having an amino acid sequence with at least 80%, at least 85%, at least 90%, at least 91%, at least 92%, at least 93%, at least 94%, at least 95%, at least 96%, at least 97%, at least 98%, at least 99%, or 100% identity to any of SEQ ID NOS: 329.

2. The immunogenic composition of claim 1, comprising from 0.1 μg to 25 μg, 0.1 μg to 10 μg, 0.001 μg to 10 μg, 0.001 μg to 25 μg, or from 0.1 μg to 5 μg of the first CoV S glycoprotoein.

3. The immunogenic composition of claim 1, comprising a nanoparticle comprising the first CoV S glycoprotein and a non-ionic detergent core.

4. The immunogenic composition of claim 3, wherein the non-ionic detergent is selected from the group consisting of polysorbate-20 (PS20), polysorbate-40 (PS40), polysorbate-60 (PS60), polysorbate-65 (PS65), and polysorbate-80 (PS80).

5. The immunogenic composition of claim 1, further comprising an adjuvant and a pharmaceutically acceptable carrier.

6. The immunogenic composition of claim 5, wherein the adjuvant is a saponin adjuvant.

7. The immunogenic composition of claim 6, wherein the saponin adjuvant comprises at least two iscom particles, wherein:
the first iscom particle comprises fraction A of Quillaja Saponaria Molina and not fraction C of Quillaja Saponaria Molina; and
the second iscom particle comprises fraction C of Quillaja Saponaria Molina and not fraction A of Quillaja Saponaria Molina.

8. The immunogenic composition of claim 5, comprising from about 25 μg to about 100 μg of adjuvant.

9. The immunogenic composition of claim 1, wherein the immunogenic composition is contained in a syringe.

10. A method of stimulating an immune response against SARS-COV-2 or a heterogeneous SARS-COV-2 strain thereof in a human comprising administering the immunogenic composition of claim 1 to the human.

11. A method of boosting an immune response against SARS-COV-2 or a heterogeneous SARS-COV-2 strain thereof in a human comprising administering:
a first immunogenic composition comprising (a) one or more first SARS-COV-2 S glycoproteins having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 329, and (b) a pharmaceutically acceptable buffer;
wherein the immunogenic composition is administered after administration of another immunogenic composition intended to produce an immunogenic response against SARS-CoV-2 or a heterogeneous SARS-COV-2 strain thereof in the human.

12. The method of claim 11, comprising administering a first dose of the first immunogenic composition at least 21 days after administration of the other immunogenic composition.

13. The method of claim 12, comprising administering the first immunogenic composition from about 1 month to about 18 months, from about 6 months to about 18 months, from about 9 months to about 18 months, from about 12 months to about 15 months, or from about 12 months to about 18 months after administration of the other immunogenic composition.

14. The method of claim 11, wherein the other immunogenic composition comprises an mRNA vaccine or a protein subunit vaccine.

15. The method of claim 14, wherein the protein subunit vaccine of the other immunogenic composition comprises at least one or more second SARS-COV-2 S glycoproteins having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any of SEQ ID NOS: 87, 260, 222, 227, 274, and 284, and 329.

16. The immunogenic composition of claim 1, wherein the first CoV S glycoprotein comprises one or more modifications compared to SEQ ID NO: 329 at amino acid 59,346,456, 475, 572, and 1087, wherein the one or more modifications are numbered according to SEQ ID NO: 1.

17. The immunogenic composition of claim 16, wherein:
(i) amino acid at 59 is phenylalanine or serine;
(ii) amino acid at 346 is arginine or threonine;
(iii) amino acid at 456 is phenylalanine or leucine;
(iv) amino acid at 475 is alanine or valine:
(v) amino acid at 572 is threonine or isoleucine; and
(vi) amino acid at 1087 is alanine or serine.

18. A method of inducing an immune response against SARS-COV-2 or a heterogeneous SARS-COV-2 strain thereof in a human comprising administering:
a first immunogenic composition comprising (a) one or more first SARS-COV-2 S glycoproteins having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 329, and (b) a pharmaceutically acceptable buffer.

19. A method as recited in claim 18, further comprising administering a second immunogenic composition, comprising (a) one or more second SARS-COV-2 S glycoproteins having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any of SEQ ID NOS: 87, 260, 222, 227, 274, and 284, and 329.

20. An immunogenic composition comprising:
(i) one or more non-naturally occurring SARS-COV-2 S glycoproteins having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NOS: 87, 260, 222, 227, 274, and 284; and
(ii) a pharmaceutically acceptable buffer.
